(19) United States
(12) Patent Application Publication
(10) Pub. No.: US 2022/0081400 A1
(43) Pub. Date: Mar. 17, 2022

(54) OPIOID HAPTENS, CONJUGATES, VACCINES, AND METHODS OF GENERATING ANTIBODIES

(71) Applicant: The Scripps Research Institute, La Jolla, CA (US)

(72) Inventors: Kim D. Janda, La Jolla, CA (US); Paul Bremer, La Jolla, CA (US); Yoshihiro Natori, La Jolla, CA (US)

(21) Appl. No.: 17/260,484

(22) PCT Filed: Jul. 16, 2019
(86) PCT No.: PCT/US2019/042083

§ 371 Date: Jan. 14, 2021

Related U.S. Application Data
(60) Provisional application No. 62/698,361, filed on Jul. 16, 2018.

(51) Int. Cl. C07D 211/66; A61K 47/64; A61K 39/00; A61K 39/385

(52) U.S. Cl. CPC C07D 211/66; A61K 47/6415; A61K 39/385; A61K 47/646; A61K 39/0013; A61K 47/643

(57) ABSTRACT

The disclosure provides, inter alia, opioid haptens, opioid hapten conjugates, opioid vaccines, methods of treating or preventing opioid use disorder, methods of treating opioid overdose, and methods of generating and/or isolating antibodies selective for opioids.

### CROSS-REFERENCES TO RELATED APPLICATIONS

This application claims priority to U.S. Application No. 62/698,361 filed Jul. 16, 2018, the disclosure of which is incorporated by reference herein in its entirety and for all purposes.

### STATEMENT AS TO RIGHTS TO INVENTIONS MADE UNDER FEDERALLY SPONSORED RESEARCH AND DEVELOPMENT

This invention was made with government support under grant no. DA041146 awarded by the National Institutes of Health. The government has certain rights in the invention.

### BACKGROUND

Every day, more than 130 people in the United States die after overdosing on opioids. The misuse of and addiction to opioids, including prescription pain relievers, heroin, and synthetic opioids such as fentanyl and carfentanil, is a serious national and global crisis that affects public health as well as social and economic welfare. The US Centers for Disease Control and Prevention estimates that the total economic burden of prescription opioid misuse alone in the United States is $78.5 billion a year, including the costs of healthcare, lost productivity, addiction treatment, and criminal justice involvement.

Current medication assisted treatments for opioid use disorder primarily rely on the use of opioids, such as methadone or buprenorphine. There is a need in the art for effective medication assisted treatments to reduce opioid addiction that do not require the use of opioids. The disclosure is directed to this, as well as other, important needs.

### BRIEF SUMMARY

The disclosure provides fentanyl haptens and carfentanil haptens. In aspects, the disclosure provides of generating and isolating antibodies using fentanyl haptens and carfentanil haptens. In aspects, the disclosure provides vaccines comprising fentanyl haptens and carfentanil haptens.

The disclosure provides fentanyl-conjugates and carfentanil-conjugates. In aspects, fentanyl is conjugated, directly or via a linking group, to a protein, a detectable moiety, an affinity moiety, a carrier, a solid support, a leaving group, a protecting group or a combination thereof. In aspects, carfentanil is conjugated, directly or via a linking group, to a protein, a detectable moiety, an affinity moiety, a carrier, a solid support, a leaving group, a protecting group or a combination thereof. In aspects, the disclosure provides methods of generating and isolating antibodies using fentanyl-conjugates or carfentanil-conjugates. In aspects, the disclosure provides vaccines comprising fentanyl conjugates or carfentanil conjugates.

These and other embodiments and aspects are described in detail herein.

### BRIEF DESCRIPTION OF THE DRAWINGS

FIGS. 1A-1C show the chemical synthesis and structures of two probes (e.g., fentanyl-conjugates) of the compounds described herein that are representative of the fentanyl-like scaffolding. FIG. 1A provides the chemical synthesis of a fentanyl hapten.

FIGS. 1B-1C provide the chemical synthesis of the fentanyl hapten linked to biotin via different linking groups.

FIGS. 2A-2B show the chemical synthesis of the compounds described herein that are representative of the carfentanil-like scaffolding. FIG. 2A provides the synthesis of a carfentanil hapten. FIG. 2B provides the chemical synthesis of a protein linked to a plurality of carfentanil or fentanyl haptens to form conjugates.

FIG. 3 shows the chemical synthesis and structures of probes (e.g., carfentanil-biotin conjugates) of the compounds described herein that are representative of the carfentanil-like scaffolding.

FIGS. 4A-4E show data relevant to one of the antibodies obtained by the methods herein. With reference to FIGS. 4A-4C, hot plate and tail flick antinociception for carfentanil (FIG. 4A) and fentanyl (FIG. 4B) was used as a surrogate for drug reward because it is mediated in the central nervous system and provides a relevant behavioral model of the antibodies ability to reduce drug access to brain and its subsequent effects. Potency ratios in FIG. 4C were calculated by dividing the vaccine-shifted ED₅₀ value from the control values in each antinociceptive test. The antibody provided complete protection from a lethal overdose of carfentanil (FIG. 4D) and fentanyl (FIG. 4E).

FIG. 5 shows the antibody midpoint titers for rats administered the CRM197-carfentanil vaccine described herein.

FIGS. 6A-6B provide the results of the antinociception assay using the hot plate and tail flick tests after the rats were intraperitoneally administered carfentanil. FIG. 6A provides the carfentanil ED₅₀ in mg/kg for rats administered the CRM197-carfentanil vaccine and control vaccine. FIG. 6B provides the carfentanil potency ratio (ED₅₀ shifts) for the CRM197-carfentanil vaccine.

FIGS. 7A-7B provide the results of the antinociception assay using the hot plate and tail flick tests after the rats were intraperitoneally administered fentanyl. FIG. 7A provides the fentanyl ED₅₀ in mg/kg for rats administered the CRM197-carfentanil vaccine and control vaccine. FIG. 7B provides the fentanyl potency ratio (ED₅₀ shifts) for the CRM197-carfentanil vaccine.

FIGS. 8A-8B provide biodistribution studies with rats administered opioids subsequent to injection with the CRM197-carfentanil vaccine. FIG. 8A provides the results of the biodistribution study of rats intraperitoneally administered 0.02 mg/kg of carfentanil. FIG. 8B provides the results of the biodistribution study of rats intraperitoneally administered 0.2 mg/kg of fentanyl.

FIGS. 9A-9B provide serum antibodies of Surface Plasma Resonance (SPR) competitive binding assay based on the structures of carfentanil, fentanyl, and fentanyl analogues. FIG. 9A provides the % binding of a carfentanil-BSA conjugate versus a control.

FIG. 9B provides the % binding of a fentanyl-BSA conjugate versus a control. For each drug category in FIGS. 9A-9B, the first bar represents bleed 1, the second bar represents bleed 2, and the third bar represents bleed 3.

### DETAILED DESCRIPTION

### Definitions

The abbreviations used herein have their conventional meaning within the chemical and biological arts. The chemical structures and formulae set forth herein are constructed according to the standard rules of chemical valency known in the chemical arts.

Where substituent groups are specified by their conventional chemical formulae, written from left to right, they equally encompass the chemically identical substituents that would result from writing the structure from right to left, e.g., —CH₂O— is equivalent to —OCH₂—.

The term “alkyl,” by itself or as part of another substituent, means, unless otherwise stated, a straight (i.e., unbranched) or branched carbon chain (or carbon), or combination thereof, which may be fully saturated, mono- or polyunsaturated and can include mono-, di- and multivalent radicals. The alkyl may include a designated number of carbons (e.g., C₁-C₁₀ means one to ten carbons). Alkyl is an uncyclized chain. Examples of saturated hydrocarbon radicals include, but are not limited to, groups such as methyl, ethyl, n-propyl, isopropyl, n-butyl, t-butyl, isobutyl, sec-butyl, methyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkoxy is an alkyl attached to the remainder of the molecule via an oxygen linker (—O—). An alkyl moiety may be an alkenyl moiety. An alkyl moiety may be an alkynyl moiety. An alkyl moiety may be fully saturated. An alkenyl may include more than one double bond and/or one or more triple bonds in addition to the one or more double bonds. An alkynyl may include more than one triple bond and/or one or more double bonds in addition to the one or more triple bonds.

The term “alkylene,” by itself or as part of another substituent, means, unless otherwise stated, a divalent radical derived from an alkyl, as exemplified by, e.g., —CH₂CH₂CH₂—. Typically, an alkyl (or alkylene) group will have from 1 to 24 carbon atoms, with those groups having 10 or fewer carbon atoms being preferred herein. A “lower alkyl” or “lower alkylene” is a shorter chain alkyl or alkylene group, generally having eight or fewer carbon atoms. The term “alkenylene,” by itself or as part of another substituent, means, unless otherwise stated, a divalent radical derived from an alkene.

The term “heteroalkyl,” by itself or in combination with another term, means, unless otherwise stated, a stable straight or branched chain, or combinations thereof, including at least one carbon atom and at least one heteroatom (e.g., O, N, P, Si, and S), and wherein the nitrogen and sulfur atoms may optionally be oxidized, and the nitrogen heteroatom may optionally be quaternized. The heteroatoms may be placed at any interior position of the heteroalkyl group or at the position at which the alkyl group is attached to the remainder of the molecule. Heteroalkyl is an uncyclized chain. Examples include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—S—CH₂, —S(O)—CH₃, —CH₂—CH₂—S(O)₂—CH₃, —CH═CHO—CH₃, —Si(CH₃)₃, —CH₂—CH═N—OCH₃, —CH═CH—N(CH₃)—CH₃, —O—CH₃, —O—CH₂—CH₃, and —CN. Up to two or three heteroatoms may be consecutive, such as, for example, —CH₂—NH—OCH₃ and —CH₂—O—Si(CH₃)₃. A heteroalkyl moiety may include one heteroatom. A heteroalkyl moiety may include two optionally different heteroatoms. A heteroalkyl moiety may include three optionally different heteroatoms. A heteroalkyl moiety may include four optionally different heteroatoms. A heteroalkyl moiety may include five optionally different heteroatoms. A heteroalkyl moiety may include up to 8 optionally different heteroatoms. The term “heteroalkenyl,” by itself or in combination with another term, means, unless otherwise stated, a heteroalkyl including at least one double bond. A heteroalkenyl may optionally include more than one double bond and/or one or more triple bonds in additional to the one or more double bonds. The term “heteroalkynyl,” by itself or in combination with another term, means, unless otherwise stated, a heteroalkyl including at least one triple bond. A heteroalkynyl may optionally include more than one triple bond and/or one or more double bonds in additional to the one or more triple bonds.

Similarly, the term “heteroalkylene,” by itself or as part of another substituent, means, unless otherwise stated, a divalent radical derived from heteroalkyl, as exemplified, but not limited by, —CH₂—CH₂—S—CH₂—CH₂—, —CH₂—S—CH₂—CH₂—NH—CH₂—, —PO₄—(CH₂)₃—PO₄, and the like. For heteroalkylene groups, heteroatoms (e.g., O, N, S, Si, or P) can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where heteroalkyl is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted as excluding specific heteroalkyl groups, such as —NR′R″ or the like.

The terms “cycloalkyl” and “heterocycloalkyl,” by themselves or in combination with other terms, mean, unless otherwise stated, cyclic versions of “alkyl” and “heteroalkyl,” respectively. Cycloalkyl and heterocycloalkyl are not aromatic. Additionally, for heterocycloalkyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene,” alone or as part of another substituent, means a divalent radical derived from a cycloalkyl and heterocycloalkyl, respectively.

In embodiments, the term “cycloalkyl” means a monocyclic, bicyclic, or a multicyclic cycloalkyl ring system. In aspects, monocyclic ring systems are cyclic hydrocarbon groups containing from 3 to 8 carbon atoms, where such groups can be saturated or unsaturated, but not aromatic. In aspects, cycloalkyl groups are fully saturated. Examples of monocyclic cycloalkyls include cyclopropyl, cyclobutyl, cyclopentyl, cyclopentenyl, cyclohexyl, cyclohexenyl, cycloheptyl, and cyclooctyl. Bicyclic cycloalkyl ring systems are bridged monocyclic rings or fused bicyclic rings. In aspects, bridged monocyclic rings contain a monocyclic cycloalkyl ring where two non adjacent carbon atoms of the monocyclic ring are linked by an alkylene bridge of between one and three additional carbon atoms (i.e., a bridging group of the form (CH₂)_{w}, where w is 1, 2, or 3). Representative examples of bicyclic ring systems include, but are not limited to, bicyclo[3.1.1]heptane, bicyclo[2.2.1]heptane, bicyclo[2.2.2]octane, bicyclo[3.2.2]nonane, bicyclo[3.3.1]nonane, and bicyclo[4.2.1]nonane. In aspects, fused bicyclic cycloalkyl ring systems contain a monocyclic cycloalkyl ring fused to either a phenyl, a monocyclic cycloalkyl, a monocyclic cycloalkenyl, a monocyclic heterocyclyl, or a monocyclic heteroaryl. In aspects, the bridged or fused bicyclic cycloalkyl is attached to the parent molecular moiety through any carbon atom contained within the monocyclic cycloalkyl ring. In aspects, cycloalkyl groups are optionally substituted with one or two groups which are independently oxo or thia. In aspects, the fused bicyclic cycloalkyl is a 5 or 6 membered monocyclic cycloalkyl ring fused to either a phenyl ring, a 5 or 6 membered monocyclic cycloalkyl, a 5 or 6 membered monocyclic cycloalkenyl, a 5 or 6 membered monocyclic heterocyclyl, or a 5 or 6 membered monocyclic heteroaryl, wherein the fused bicyclic cycloalkyl is optionally substituted by one or two groups which are independently oxo or thia. In aspects, multicyclic cycloalkyl ring systems are a monocyclic cycloalkyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two other ring systems independently selected from the group consisting of a phenyl, a bicyclic aryl, a monocyclic or bicyclic heteroaryl, a monocyclic or bicyclic cycloalkyl, a monocyclic or bicyclic cycloalkenyl, and a monocyclic or bicyclic heterocyclyl. In aspects, the multicyclic cycloalkyl is attached to the parent molecular moiety through any carbon atom contained within the base ring. In aspects, multicyclic cycloalkyl ring systems are a monocyclic cycloalkyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two other ring systems independently selected from the group consisting of a phenyl, a monocyclic heteroaryl, a monocyclic cycloalkyl, a monocyclic cycloalkenyl, and a monocyclic heterocyclyl. Examples of multicyclic cycloalkyl groups include, but are not limited to tetradecahydrophenanthrenyl, perhydrophenothiazin-1-yl, and perhydrophenoxazin-1-yl.

In embodiments, a cycloalkyl is a cycloalkenyl. The term “cycloalkenyl” is used in accordance with its plain ordinary meaning. In aspects, a cycloalkenyl is a monocyclic, bicyclic, or a multicyclic cycloalkenyl ring system. In aspects, monocyclic cycloalkenyl ring systems are cyclic hydrocarbon groups containing from 3 to 8 carbon atoms, where such groups are unsaturated (i.e., containing at least one annular carbon carbon double bond), but not aromatic. Examples of monocyclic cycloalkenyl ring systems include cyclopentenyl and cyclohexenyl. In aspects, bicyclic cycloalkenyl rings are bridged monocyclic rings or a fused bicyclic rings. In aspects, bridged monocyclic rings contain a monocyclic cycloalkenyl ring where two non adjacent carbon atoms of the monocyclic ring are linked by an alkylene bridge of between one and three additional carbon atoms (i.e., a bridging group of the form (CH₂)_{w}, where w is 1, 2, or 3). Representative examples of bicyclic cycloalkenyls include, but are not limited to, norbornenyl and bicyclo[2.2.2]oct 2 enyl. In aspects, fused bicyclic cycloalkenyl ring systems contain a monocyclic cycloalkenyl ring fused to either a phenyl, a monocyclic cycloalkyl, a monocyclic cycloalkenyl, a monocyclic heterocyclyl, or a monocyclic heteroaryl. In aspects, the bridged or fused bicyclic cycloalkenyl is attached to the parent molecular moiety through any carbon atom contained within the monocyclic cycloalkenyl ring. In aspects, cycloalkenyl groups are optionally substituted with one or two groups which are independently oxo or thia. In aspects, multicyclic cycloalkenyl rings contain a monocyclic cycloalkenyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two ring systems independently selected from the group consisting of a phenyl, a bicyclic aryl, a monocyclic or bicyclic heteroaryl, a monocyclic or bicyclic cycloalkyl, a monocyclic or bicyclic cycloalkenyl, and a monocyclic or bicyclic heterocyclyl. In aspects, the multicyclic cycloalkenyl is attached to the parent molecular moiety through any carbon atom contained within the base ring. In aspects, multicyclic cycloalkenyl rings contain a monocyclic cycloalkenyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two ring systems independently selected from the group consisting of a phenyl, a monocyclic heteroaryl, a monocyclic cycloalkyl, a monocyclic cycloalkenyl, and a monocyclic heterocyclyl.

In embodiments, a heterocycloalkyl is a heterocyclyl. The term “heterocyclyl” as used herein, means a monocyclic, bicyclic, or multicyclic heterocycle. The heterocyclyl monocyclic heterocycle is a 3, 4, 5, 6 or 7 membered ring containing at least one heteroatom independently selected from the group consisting of O, N, and S where the ring is saturated or unsaturated, but not aromatic. The 3 or 4 membered ring contains 1 heteroatom selected from the group consisting of O, N and S. The 5 membered ring can contain zero or one double bond and one, two or three heteroatoms selected from the group consisting of O, N and S.

The 6 or 7 membered ring contains zero, one or two double bonds and one, two or three heteroatoms selected from the group consisting of O, N and S. The heterocyclyl monocyclic heterocycle is connected to the parent molecular moiety through any carbon atom or any nitrogen atom contained within the heterocyclyl monocyclic heterocycle. Representative examples of heterocyclyl monocyclic heterocycles include, but are not limited to, azetidinyl, azepanyl, aziridinyl, diazepanyl, 1,3-dioxanyl, 1,3-dioxolanyl, 1,3-dithiolanyl, 1,3-dithianyl, imidazolinyl, imidazolidinyl, isothiazolinyl, isothiazolidinyl, isoxazolinyl, isoxazolidinyl, morpholinyl, oxadiazolinyl, oxadiazolidinyl, oxazolinyl, oxazolidinyl, piperazinyl, piperidinyl, pyranyl, pyrazolinyl, pyrazolidinyl, pyrrolinyl, pyrrolidinyl, tetrahydrofuranyl, tetrahydrothienyl, thiadiazolinyl, thiadiazolidinyl, thiazolinyl, thiazolidinyl, thiomorpholinyl, 1,1-dioxidothiomorpholinyl (thiomorpholine sulfone), thiopyranyl, and trithianyl. The heterocyclyl bicyclic heterocycle is a monocyclic heterocycle fused to either a phenyl, a monocyclic cycloalkyl, a monocyclic cycloalkenyl, a monocyclic heterocycle, or a monocyclic heteroaryl. The heterocyclyl bicyclic heterocycle is connected to the parent molecular moiety through any carbon atom or any nitrogen atom contained within the monocyclic heterocycle portion of the bicyclic ring system. Representative examples of bicyclic heterocyclyls include, but are not limited to, 2,3-dihydrobenzofuran-2-yl, 2,3-dihydrobenzofuran-3-yl, indolin-1-yl, indolin-2-yl, indolin-3-yl, 2,3-dihydrobenzothien-2-yl, decahydroquinolinyl, decahydroisoquinolinyl, octahydro-1H-indolyl, and octahydrobenzofuranyl. In aspects, heterocyclyl groups are optionally substituted with one or two groups which are independently oxo or thia. In aspects, the bicyclic heterocyclyl is a 5 or 6 membered monocyclic heterocyclyl ring fused to a phenyl ring, a 5 or 6 membered monocyclic cycloalkyl, a 5 or 6 membered monocyclic cycloalkenyl, a 5 or 6 membered monocyclic heterocyclyl, or a 5 or 6 membered monocyclic heteroaryl, wherein the bicyclic heterocyclyl is optionally substituted by one or two groups which are independently oxo or thia. Multicyclic heterocyclyl ring systems are a monocyclic heterocyclyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two other ring systems independently selected from the group consisting of a phenyl, a bicyclic aryl, a monocyclic or bicyclic heteroaryl, a monocyclic or bicyclic cycloalkyl, a monocyclic or bicyclic cycloalkenyl, and a monocyclic or bicyclic heterocyclyl. The multicyclic heterocyclyl is attached to the parent molecular moiety through any carbon atom or nitrogen atom contained within the base ring. In aspects, multicyclic heterocyclyl ring systems are a monocyclic heterocyclyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two other ring systems independently selected from the group consisting of a phenyl, a monocyclic heteroaryl, a monocyclic cycloalkyl, a monocyclic cycloalkenyl, and a monocyclic heterocyclyl. Examples of multicyclic heterocyclyl groups include, but are not limited to 10H-phenothiazin-10-yl, 9,10-dihydroacridin-9-yl, 9,10-dihydroacridin-10-yl, 10H-phenoxazin-10-yl, 10,11-dihydro-5H-dibenzo[b,f]azepin-5-yl, 1,2,3,4-tetrahydropyrido[4,3-g]isoquinolin-2-yl, 12H-benzo[b]phenoxazin-12-yl, and dodecahydro-1H-carbazol-9-yl.

The terms “halo” or “halogen,” by themselves or as part of another substituent, mean, unless otherwise stated, a fluorine, chlorine, bromine, or iodine atom. Additionally, terms such as “haloalkyl” are meant to include monohaloalkyl and polyhaloalkyl. For example, the term “halo(C₁-C₄)alkyl” includes, but is not limited to, fluoromethyl, difluoromethyl, trifluoromethyl, 2,2,2-trifluoroethyl, 4-chlorobutyl, 3-bromopropyl, and the like.

The term “acyl” means, unless otherwise stated, —C(O)R where R is a substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl.

The term “aryl” means, unless otherwise stated, a polyunsaturated, aromatic, hydrocarbon substituent, which can be a single ring or multiple rings (preferably from 1 to 3 rings) that are fused together (i.e., a fused ring aryl) or linked covalently. A fused ring aryl refers to multiple rings fused together wherein at least one of the fused rings is an aryl ring. The term “heteroaryl” refers to aryl groups (or rings) that contain at least one heteroatom such as N, O, or S, wherein the nitrogen and sulfur atoms are optionally oxidized, and the nitrogen atom(s) are optionally quaternized. Thus, the term “heteroaryl” includes fused ring heteroaryl groups (i.e., multiple rings fused together wherein at least one of the fused rings is a heteroaromatic ring). A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. And a 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom. Non-limiting examples of aryl and heteroaryl groups include phenyl, naphthyl, pyrrolyl, pyrazolyl, pyridazinyl, triazinyl, pyrimidinyl, imidazolyl, pyrazinyl, purinyl, oxazolyl, isoxazolyl, thiazolyl, furyl, thienyl, pyridyl, pyrimidyl, benzothiazolyl, benzoxazoyl benzimidazolyl, benzofuran, isobenzofuranyl, indolyl, isoindolyl, benzothiophenyl, isoquinolyl, quinoxalinyl, quinolyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. An “arylene” and a “heteroarylene,” alone or as part of another substituent, mean a divalent radical derived from an aryl and heteroaryl, respectively. A heteroaryl group substituent may be —O— bonded to a ring heteroatom nitrogen.

A fused ring heterocyloalkyl-aryl is an aryl fused to a heterocycloalkyl. A fused ring heterocycloalkyl-heteroaryl is a heteroaryl fused to a heterocycloalkyl. A fused ring heterocycloalkyl-cycloalkyl is a heterocycloalkyl fused to a cycloalkyl. A fused ring heterocycloalkyl-heterocycloalkyl is a heterocycloalkyl fused to another heterocycloalkyl. Fused ring heterocycloalkyl-aryl, fused ring heterocycloalkyl-heteroaryl, fused ring heterocycloalkyl-cycloalkyl, or fused ring heterocycloalkyl-heterocycloalkyl may each independently be unsubstituted or substituted with one or more of the substitutents described herein.

Spirocyclic rings are two or more rings wherein adjacent rings are attached through a single atom. The individual rings within spirocyclic rings may be identical or different. Individual rings in spirocyclic rings may be substituted or unsubstituted and may have different substituents from other individual rings within a set of spirocyclic rings. Possible substituents for individual rings within spirocyclic rings are the possible substituents for the same ring when not part of spirocyclic rings (e.g. substituents for cycloalkyl or heterocycloalkyl rings). Spirocylic rings may be substituted or unsubstituted cycloalkyl, substituted or unsubstituted cycloalkylene, substituted or unsubstituted heterocycloalkyl or substituted or unsubstituted heterocycloalkylene and individual rings within a spirocyclic ring group may be any of the immediately previous list, including having all rings of one type (e.g. all rings being substituted heterocycloalkylene wherein each ring may be the same or different substituted heterocycloalkylene). When referring to a spirocyclic ring system, heterocyclic spirocyclic rings means a spirocyclic rings wherein at least one ring is a heterocyclic ring and wherein each ring may be a different ring. When referring to a spirocyclic ring system, substituted spirocyclic rings means that at least one ring is substituted and each substituent may optionally be different.

The symbol “ [symbol] ” or “-” denotes the point of attachment of a chemical moiety to the remainder of a molecule or chemical formula.

The term “oxo” means an oxygen that is double bonded to a carbon atom.

The term “alkylsulfonyl,” as used herein, means a moiety having the formula —S(O₂)—R′, where R′ is a substituted or unsubstituted alkyl group as defined above. R′ may have a specified number of carbons (e.g., “C₁-C₄ alkylsulfonyl”).

The term “alkylarylene” as an arylene moiety covalently bonded to an alkylene moiety (also referred to herein as an alkylene linker). In aspects, the alkylarylene group has the formula:

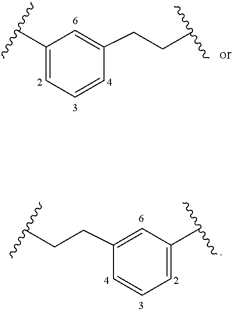 [SMILES: *CCc1cccc(*)c1 ; *CCc1cccc(*)c1]

An alkylarylene moiety may be substituted (e.g. with a substituent group) on the alkylene moiety or the arylene linker (e.g. at carbons 2, 3, 4, or 6) with halogen, oxo, —N₃, —CF₃, —CCl₃, —CBr₃, —Cl₃, —CN, —CHO, —OH, —NH₂, —COOH, —CONH₂, —NO₂, —SH, —SO₂CH₃—SO₃H, —OSO₃H, —SO₂NH₂, —NHNH₂, —ONH₂, —NHC(O)NHNH₂, substituted or unsubstituted C₁-C₅ alkyl or substituted or unsubstituted 2 to 5 membered heteroalkyl). In aspects, the alkylarylene is unsubstituted.

Each of the above terms (e.g., “alkyl,” “heteroalkyl,” “cycloalkyl,” “heterocycloalkyl,” “aryl,” and “heteroaryl”) includes both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to, —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR′″, —NR—C(NR′R″)═NR′″, —S(O)R′, —S(O)₂R′, —S(O)₂NR′R″, —NRSO₂R′, —NR′NR″R′″, —ONR′R″, —NR′C(O)NR″NR′″R′″, —CN, —NO₂, —NR′SO₂R″, —NR′C(O)R″, —NR′C(O)—OR″, —NR′OR″, in a number ranging from zero to (2m′+1), where m′ is the total number of carbon atoms in such radical. R, R′, R″, R′″, and R′″ each preferably independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted heteroaryl, substituted or unsubstituted alkyl, alkoxy, or thioalkoxy groups, or arylalkyl groups. When a compound described herein includes more than one R group, for example, each of the R groups is independently selected as are each R′, R″, R′″, and R′″ group when more than one of these groups is present. When R′ and R″ are attached to the same nitrogen atom, they can be combined with the nitrogen atom to form a 4-, 5-, 6-, or 7-membered ring. For example, —NR′R″ includes, but is not limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example: —OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR′″, —NR—C(NR′R″)═NR′″, —S(O)R′, —S(O)₂R′, —S(O)₂NR′R″, —NRSO₂R′, —NR′NR″R′″, —ONR′R″, —NR′C(O)NR″NR′″R′″, —CN, —NO₂, —R′, —N₃, —CH(Ph)₂, fluoro(C₁-C₄)alkoxy, and fluoro(C₁-C₄)alkyl, —NR′SO₂R″, —NR′C(O)R″, —NR′C(O)—OR″, —NR′OR″, in a number ranging from zero to the total number of open valences on the aromatic ring system; and where R′, R″, R′″, and R′″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. When a compound described herein includes more than one R group, for example, each of the R groups is independently selected as are each R′, R″, R′″, and R′″ groups when more than one of these groups is present.

Substituents for rings (e.g. cycloalkyl, heterocycloalkyl, aryl, heteroaryl, cycloalkylene, heterocycloalkylene, arylene, or heteroarylene) may be depicted as substituents on the ring rather than on a specific atom of a ring (commonly referred to as a floating substituent). In such a case, the substituent may be attached to any of the ring atoms (obeying the rules of chemical valency) and in the case of fused rings or spirocyclic rings, a substituent depicted as associated with one member of the fused rings or spirocyclic rings (a floating substituent on a single ring), may be a substituent on any of the fused rings or spirocyclic rings (a floating substituent on multiple rings). When a substituent is attached to a ring, but not a specific atom (a floating substituent), and a subscript for the substituent is an integer greater than one, the multiple substituents may be on the same atom, same ring, different atoms, different fused rings, different spirocyclic rings, and each substituent may optionally be different. Where a point of attachment of a ring to the remainder of a molecule is not limited to a single atom (a floating substituent), the attachment point may be any atom of the ring and in the case of a fused ring or spirocyclic ring, any atom of any of the fused rings or spirocyclic rings while obeying the rules of chemical valency. Where a ring, fused rings, or spirocyclic rings contain one or more ring heteroatoms and the ring, fused rings, or spirocyclic rings are shown with one more floating substituents (including, but not limited to, points of attachment to the remainder of the molecule), the floating substituents may be bonded to the heteroatoms. Where the ring heteroatoms are shown bound to one or more hydrogens (e.g. a ring nitrogen with two bonds to ring atoms and a third bond to a hydrogen) in the structure or formula with the floating substituent, when the heteroatom is bonded to the floating substituent, the substituent will be understood to replace the hydrogen, while obeying the rules of chemical valency.

Two or more substituents may optionally be joined to form aryl, heteroaryl, cycloalkyl, or heterocycloalkyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

Two of the substituents on adjacent atoms of the aryl or heteroaryl ring may optionally form a ring of the formula -T-C(O)—(CRR′)_{q}-U-, wherein T and U are independently —NR—, —O—, —CRR′—, or a single bond, and q is an integer of from 0 to 3. Alternatively, two of the substituents on adjacent atoms of the aryl or heteroaryl ring may optionally be replaced with a substituent of the formula -A-(CH₂)ᵣ-B-, wherein A and B are independently —CRR′—, —O—, —NR—, —S—, —S(O)—, —S(O)₂—, —S(O)₂NR′—, or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X′—(C″R″R′″)_{d}—, where s and d are independently integers of from 0 to 3, and X′ is —O—, —NR′—, —S—, —S(O)—, —S(O)₂—, or —S(O)₂NR′—. The substituents R, R′, R″, and R′″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

As used herein, the terms “heteroatom” or “ring heteroatom” are meant to include oxygen (O), nitrogen (N), sulfur (S), phosphorus (P), and silicon (Si).

A “substituent group,” as used herein, means a group selected from the following moieties: (A) oxo, halogen, —CCl₃, —CBr₃, —CF₃, —Cl₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, —N₃ unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and (B) alkyl, heteroalkyl, cycloalkyl, heterocycloalkyl, aryl, heteroaryl, substituted with at least one substituent selected from: (i) oxo, halogen, —CCl₃, —CBr₃, —CF₃, —Cl₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, —N₃ unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and (ii) alkyl, heteroalkyl, cycloalkyl, heterocycloalkyl, aryl, heteroaryl, substituted with at least one substituent selected from: (a) oxo, halogen, —CCl₃, —CBr₃, —CF₃, —Cl₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, —N₃, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and (b) alkyl, heteroalkyl, cycloalkyl, heterocycloalkyl, aryl, heteroaryl, substituted with at least one substituent selected from: oxo, halogen, —CCl₃, —CBr₃, —CF₃, —Cl₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, —N₃, unsubstituted alkyl (e.g., C₁-C₅ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

A “size-limited substituent” or “size-limited substituent group,” as used herein, means a group selected from all of the substituents described above for a “substituent group,” wherein each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and each substituted or unsubstituted heteroaryl is a substituted or unsubstituted 5 to 10 membered heteroaryl.

A “lower substituent” or “lower substituent group,” as used herein, means a group selected from all of the substituents described above for a “substituent group,” wherein each substituted or unsubstituted alkyl is a substituted or unsubstituted C₁-C₅ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and each substituted or unsubstituted heteroaryl is a substituted or unsubstituted 5 to 9 membered heteroaryl.

In embodiments, each substituted group described in the compounds herein is substituted with at least one substituent group. In aspects, each substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene described in the compounds herein are substituted with at least one substituent group. In aspects, at least one or all of these groups are substituted with at least one size-limited substituent group. In aspects, at least one or all of these groups are substituted with at least one lower substituent group.

In embodiments, each substituted or unsubstituted alkyl may be a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 10 membered heteroaryl. In aspects, each substituted or unsubstituted alkylene is a substituted or unsubstituted C₁-C₂₀ alkylene, each substituted or unsubstituted heteroalkylene is a substituted or unsubstituted 2 to 20 membered heteroalkylene, each substituted or unsubstituted cycloalkylene is a substituted or unsubstituted C₃-C₈ cycloalkylene, each substituted or unsubstituted heterocycloalkylene is a substituted or unsubstituted 3 to 8 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 10 membered heteroarylene.

In embodiments, each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 9 membered heteroaryl. In aspects, each substituted or unsubstituted alkylene is a substituted or unsubstituted C₁-C₈ alkylene, each substituted or unsubstituted heteroalkylene is a substituted or unsubstituted 2 to 8 membered heteroalkylene, each substituted or unsubstituted cycloalkylene is a substituted or unsubstituted C₃-C₇ cycloalkylene, each substituted or unsubstituted heterocycloalkylene is a substituted or unsubstituted 3 to 7 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 9 membered heteroarylene. In aspects, the compound is a chemical species set forth in the Examples section, figures, or tables below.

In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is unsubstituted (e.g., is an unsubstituted alkyl, unsubstituted heteroalkyl, unsubstituted cycloalkyl, unsubstituted heterocycloalkyl, unsubstituted aryl, unsubstituted heteroaryl, unsubstituted alkylene, unsubstituted heteroalkylene, unsubstituted cycloalkylene, unsubstituted heterocycloalkylene, unsubstituted arylene, and/or unsubstituted heteroarylene, respectively). In aspects, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is substituted (e.g., is a substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene, respectively).

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, wherein if the substituted moiety is substituted with a plurality of substituent groups, each substituent group may optionally be different. In aspects, if the substituted moiety is substituted with a plurality of substituent groups, each substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one size-limited substituent group, wherein if the substituted moiety is substituted with a plurality of size-limited substituent groups, each size-limited substituent group may optionally be different. In aspects, if the substituted moiety is substituted with a plurality of size-limited substituent groups, each size-limited substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one lower substituent group, wherein if the substituted moiety is substituted with a plurality of lower substituent groups, each lower substituent group may optionally be different. In aspects, if the substituted moiety is substituted with a plurality of lower substituent groups, each lower substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted moiety is substituted with a plurality of groups selected from substituent groups, size-limited substituent groups, and lower substituent groups; each substituent group, size-limited substituent group, and/or lower substituent group may optionally be different. In aspects, if the substituted moiety is substituted with a plurality of groups selected from substituent groups, size-limited substituent groups, and lower substituent groups; each substituent group, size-limited substituent group, and/or lower substituent group is different.

Where a moiety is substituted (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene), the moiety is substituted with at least one substituent (e.g., a substituent group, a size-limited substituent group, or lower substituent group) and each substituent is optionally different. Additionally, where multiple substituents are present on a moiety, each substituent may be optionally differently.

Certain compounds of the present disclosure possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)- or (S)- or, as (D)- or (L)- for amino acids, and individual isomers are encompassed within the scope of the present disclosure. The compounds of the present disclosure do not include those that are known in art to be too unstable to synthesize and/or isolate. The present disclosure is meant to include compounds in racemic and optically pure forms. Optically active (R)- and (S)-, or (D)- and (L)-isomers may be prepared using chiral synthons or chiral reagents, or resolved using conventional techniques. When the compounds described herein contain olefinic bonds or other centers of geometric asymmetry, and unless specified otherwise, it is intended that the compounds include both E and Z geometric isomers.

As used herein, the term “isomers” refers to compounds having the same number and kind of atoms, and hence the same molecular weight, but differing in respect to the structural arrangement or configuration of the atoms. Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the disclosure. The term “tautomer,” as used herein, refers to one of two or more structural isomers which exist in equilibrium and which are readily converted from one isomeric form to another. It will be apparent to one skilled in the art that certain compounds of this disclosure may exist in tautomeric forms, all such tautomeric forms of the compounds being within the scope of the disclosure.

It should be noted that throughout the application that alternatives are written in Markush groups, for example, each amino acid position that contains more than one possible amino acid. It is specifically contemplated that each member of the Markush group should be considered separately, thereby comprising another embodiment, and the Markush group is not to be read as a single unit.

As used herein, the term “bioconjugate reactive moiety” and “bioconjugate” refers to the resulting association between atoms or molecules of bioconjugate reactive groups. The association can be direct or indirect. For example, a conjugate between a first bioconjugate reactive group (e.g., —NH2, —COOH, —N-hydroxysuccinimide, or -maleimide) and a second bioconjugate reactive group (e.g., sulfhydryl, sulfur-containing amino acid, amine, amine sidechain containing amino acid, or carboxylate) provided herein can be direct, e.g., by covalent bond or linker (e.g. a first linker of second linker), or indirect, e.g., by non-covalent bond (e.g. electrostatic interactions (e.g. ionic bond, hydrogen bond, halogen bond), van der Waals interactions (e.g. dipole-dipole, dipole-induced dipole, London dispersion), ring stacking (pi effects), hydrophobic interactions and the like). In aspects, bioconjugates or bioconjugate linkers are formed using bioconjugate chemistry (i.e. the association of two bioconjugate reactive groups) including, but are not limited to nucleophilic substitutions (e.g., reactions of amines and alcohols with acyl halides, active esters), electrophilic substitutions (e.g., enamine reactions) and additions to carbon-carbon and carbon-heteroatom multiple bonds (e.g., Michael reaction, Diels-Alder addition). These and other useful reactions are discussed in, for example, March, Advanced Organic Chemistry, 3rd Ed., John Wiley & Sons, New York, 1985; Hermanson, Bioconjugate Techniques, Academic Press, San Diego, 1996; and Feeney et al., Modification of Proteins; Advances in Chemistry Series, Vol. 198, American Chemical Society, Washington, D.C. , 1982. In aspects, the first bioconjugate reactive group (e.g., maleimide moiety) is covalently attached to the second bioconjugate reactive group (e.g. a sulfhydryl). In aspects, the first bioconjugate reactive group (e.g., haloacetyl moiety) is covalently attached to the second bioconjugate reactive group (e.g. a sulfhydryl). In aspects, the first bioconjugate reactive group (e.g., pyridyl moiety) is covalently attached to the second bioconjugate reactive group (e.g. a sulfhydryl). In aspects, the first bioconjugate reactive group (e.g., —N-hydroxysuccinimide moiety) is covalently attached to the second bioconjugate reactive group (e.g. an amine). In aspects, the first bioconjugate reactive group (e.g., maleimide moiety) is covalently attached to the second bioconjugate reactive group (e.g. a sulfhydryl). In aspects, the first bioconjugate reactive group (e.g., -sulfo-N-hydroxysuccinimide moiety) is covalently attached to the second bioconjugate reactive group (e.g. an amine).

Useful bioconjugate reactive moieties used for bioconjugate chemistries to prepare the compounds described herein include, for example: (a) carboxyl groups and various derivatives thereof including, but not limited to, N-hydroxysuccinimide esters, N-hydroxybenztriazole esters, acid halides, acyl imidazoles, thioesters, p-nitrophenyl esters, alkyl, alkenyl, alkynyl and aromatic esters; (b) hydroxyl groups which can be converted to esters, ethers, aldehydes, etc; (c) haloalkyl groups wherein the halide can be later displaced with a nucleophilic group such as, for example, an amine, a carboxylate anion, thiol anion, carbanion, or an alkoxide ion, thereby resulting in the covalent attachment of a new group at the site of the halogen atom; (d) dienophile groups which are capable of participating in Diels-Alder reactions such as, for example, maleimido or maleimide groups; (e) aldehyde or ketone groups such that subsequent derivatization is possible via formation of carbonyl derivatives such as, for example, imines, hydrazones, semicarbazones or oximes, or via such mechanisms as Grignard addition or alkyllithium addition; (f) sulfonyl halide groups for subsequent reaction with amines, for example, to form sulfonamides; (g) thiol groups, which can be converted to disulfides, reacted with acyl halides, or bonded to metals such as gold, or react with maleimides; (h) amine or sulfhydryl groups (e.g., present in cysteine), which can be, for example, acylated, alkylated or oxidized; (i) alkenes, which can undergo, for example, cycloadditions, acylation, Michael addition, etc; (j) epoxides, which can react with, for example, amines and hydroxyl compounds; (k) phosphoramidites and other standard functional groups useful in nucleic acid synthesis; (l) metal silicon oxide bonding; (m) metal bonding to reactive phosphorus groups (e.g. phosphines) to form, for example, phosphate diester bonds; (n) azides coupled to alkynes using copper catalyzed cycloaddition click chemistry; and (o) biotin conjugate can react with avidin or strepavidin to form a avidin-biotin complex or streptavidin-biotin complex.

The bioconjugate reactive groups can be chosen such that they do not participate in, or interfere with, the chemical stability of the conjugate described herein. Alternatively, a reactive functional group can be protected from participating in the crosslinking reaction by the presence of a protecting group. In aspects, the bioconjugate comprises a molecular entity derived from the reaction of an unsaturated bond, such as a maleimide, and a sulfhydryl group.

“Analog,” or “analogue” is used in accordance with its plain ordinary meaning within chemistry and biology and refer to a chemical compound that is structurally similar to another compound (i.e., a so-called “reference” compound) but differ in composition, e.g., in the replacement of one atom by an atom of a different element, or in the presence of a particular functional group, or the replacement of one functional group by another functional group, or the absolute stereochemistry of one or more chiral centers of the reference compound. Accordingly, an analog is a compound that is similar or comparable in function and appearance but not in structure to a reference compound.

“Carfentanil analogue” refers to an analogue of carfentanil. In aspects, a carfentanil analogue is a compound that exhibits mu-opioid receptor binding greater than carfentanil or that exhibits mu-opioid receptor binding in an amount of about 0-25% less than fentanyl, or about 0-10% less than carfentanil, based on a standard in vitro or in vivo mu-opioid receptor binding assay. Exemplary carfentanil analogues include sufentanil, remifentanil, alfentanil, lofentanil, brifentanil, trefentanil, and the like. In aspects, the carfentanil analogue is sufentanil. In aspects, the carfentanil analogue is remifentanil. In aspects, the carfentanil analogue is alfentanil. In aspects, the carfentanil analogue is lofentanil. In aspects, the carfentanil analogue is brifentanil. In aspects, the carfentanil analogue is trefentanil.

“Fentanyl analogue” refers to an analogue of fentanyl. In aspects, a fentanyl analogue is a compound that exhibits mu-opioid receptor binding greater than fentanyl or that exhibits mu-opioid receptor binding in an amount of about 0-25% less than fentanyl, or about 0-10% less than fentanyl, based on standard in vitro or in vivo mu-opioid receptor binding assay. Exemplary fentanyl analogues include acetylfentanyl, butyrfentanyl, para-tolylfentanyl, 3-methylfentanyl, α-methylfentanyl, mefentanyl, phenaridine, ohmefentanyl, mirfentanil, and the like. In aspects, the fentanyl analogue is acetylfentanyl. In aspects, the fentanyl analogue is butyrfentanyl. In aspects, the fentanyl analogue is para-tolylfentanyl. In aspects, the fentanyl analogue is 3-methylfentanyl. In aspects, the fentanyl analogue is α-methylfentanyl. In aspects, the fentanyl analogue is mefentanyl. In aspects, the fentanyl analogue is phenaridine. In aspects, the fentanyl analogue is ohmefentanyl. In aspects, the fentanyl analogue is mirfentanil.

The terms “a” or “an,” as used in herein means one or more. In addition, the phrase “substituted with a[n],” as used herein, means the specified group may be substituted with one or more of any or all of the named substituents. For example, where a group, such as an alkyl or heteroaryl group, is “substituted with an unsubstituted C₁-C₂₀ alkyl, or unsubstituted 2 to 20 membered heteroalkyl,” the group may contain one or more unsubstituted C₁-C₂₀ alkyls, and/or one or more unsubstituted 2 to 20 membered heteroalkyls.

Where a moiety is substituted with an R substituent, the group may be referred to as “R-substituted.” Where a moiety is R-substituted, the moiety is substituted with at least one R substituent and each R substituent is optionally different. Where a particular R group is present in the description of a chemical genus, a Roman alphabetic symbol may be used to distinguish each appearance of that particular R group. For example, where multiple R¹³ substituents are present, each R¹³ substituent may be distinguished as R^{13A}, R^{13B}, R^{13C}, R^{13D} etc., wherein each of R^{13A}, R^{13B}, R^{13C}, R^{13D}, etc. is defined within the scope of the definition of R¹³ and optionally differently.

The term “protein” refers to a polymer of amino acid residues, wherein the polymer may be conjugated to a moiety that does not consist of amino acids. The term applies to amino acid polymers in which one or more amino acid residue is an artificial chemical mimetic of a corresponding naturally occurring amino acid, as well as to naturally occurring amino acid polymers and non-naturally occurring amino acid polymers. When the protein (e.g., X¹) is linked to a carfentanil hapten or fentanyl hapten (e.g., directly or via an L¹ group), the skilled artisan will appreciate that the protein will be a monovalent protein that allows for covalent attachment of the protein directly to the carfentanil hapten or fentanyl hapten or that allows for covalent attachment of the protein to a linking group that is covalently attached to the carfentanil hapten or fentanyl hapten. Exemplary proteins include keyhole limpet hemocyanin, albumin (e.g., bovine serum albumin, human serum albumin), tetanus toxoid, CRM197, diphtheria toxoid, Pseudomonas aeruginosa exoprotein A, cholera toxin subunit b, flagellin, and the like.

A “detectable agent” or “detectable moiety” is a composition detectable by appropriate means such as spectroscopic, photochemical, biochemical, immunochemical, chemical, magnetic resonance imaging, or other physical means. A detectable moiety is a monovalent detectable agent or a detectable agent bound (e.g. covalently and directly or via a linking group) with another compound, e.g., a hapten. Exemplary detectable agents/moieties for use in the present disclosure include an antibody ligand, a peptide, a nucleic acid, radioisotopes, paramagnetic metal ions, fluorophore (e.g. fluorescent dyes), electron-dense reagents, enzymes (e.g., as commonly used in an ELISA), biotin, a biotin-avidin complex, a biotin-streptavidin complex, digoxigenin, magnetic beads (e.g., DYNABEADS® by ThermoFisher, encompassing functionalized magnetic beads such as DYNABEADS® M-270 amine by ThermoFisher), paramagnetic molecules, paramagnetic nanoparticles, ultrasmall superparamagnetic iron oxide nanoparticles, ultrasmall superparamagnetic iron oxide nanoparticle aggregates, superparamagnetic iron oxide nanoparticles, superparamagnetic iron oxide nanoparticle aggregates, monocrystalline iron oxide nanoparticles, monochrystalline iron oxide, nanoparticle contrast agents, liposomes or other delivery vehicles containing Gadolinium chelate molecules, gadolinium, radionuclides (e.g. carbon-11, nitrogen-13, oxygen-15, fluorine-18, rubidium-82), fluorodeoxyglucose (e.g. fluorine-18 labeled), any gamma ray emitting radionuclides, positron-emitting radionuclide, radiolabeled glucose, radiolabeled water, radiolabeled ammonia, biocolloids, microbubbles (e.g. including microbubble shells including albumin, galactose, lipid, and/or polymers; microbubble gas core including air, heavy gas(es), perfluorcarbon, nitrogen, octafluoropropane, perflexane lipid microsphere, perflutren, etc.), iodinated contrast agents (e.g. iohexol, iodixanol, ioversol, iopamidol, ioxilan, iopromide, diatrizoate, metrizoate, ioxaglate), barium sulfate, thorium dioxide, gold, gold nanoparticles, gold nanoparticle aggregates, fluorophores, two-photon fluorophores, or haptens and proteins or other entities which can be made detectable, e.g., by incorporating a radiolabel into a peptide or antibody specifically reactive with a target peptide. In aspects, the detectable agent is a detectable fluorescent agent. In aspects, the detectable agent is a detectable phosphorescent agent. In aspects, the detectable agent is a detectable radioactive agent. In aspects, the detectable agent is a detectable metalloenzyme. In aspects, the detectable agent is a detectable colorimetric agent. In aspects, the detectable agent is a detectable luminescent agent. In aspects, the detectable agent is a detectable spectrophotometric agent. In aspects, the detectable agent is a detectable metal-organic framework. In aspects, the detectable agent is not an unsubstituted alkyl or hydrogen. In aspects, the detectable agent is not a radioactive isotopic atom. In aspects, the detectable agent is not a radionuclide. In aspects, the detectable agent is detectable by means other than by spectroscopy. In aspects, the detectable agent comprises a fluorophore linked to biotin, avidin, or streptavidin. In aspects, the detectable agent comprises a fluorophore linked to streptavidin. In aspects, the detectable agent comprises a fluorophore linked to avidin. In aspects, the detectable agent comprises a fluorophore linked to avidin linked to biotin. In aspects, the detectable agent comprises a fluorophore linked to streptavidin linked to biotin.

An “affinity agent” or “affinity moiety” is a compound or composition used in the separation or isolation of another compound, e.g., a hapten. An affinity moiety is a monovalent affinity agent or affinity agent bound (e.g. covalently and directly or via a linking group) with another compound, e.g., a hapten. An affinity agent/moiety may be employed in a laboratory technique known as affinity chromatography, used in the separation of biochemical mixtures by exploiting molecular and biological properties. In aspects, the affinity agent forms one half of a binding pair, wherein the binding pair includes the affinity agent and an affinity agent ligand. In aspects, the affinity agent and the affinity agent ligand non-covalently and specifically bind to one another at a low dissociation rate (e.g. in the nanomolar range). In aspects, the affinity agent and the affinity agent ligand bind together with a dissociation constant in the picomolar range (e.g. less than 1 nM). Exemplary affinity agents/moieties for use in the present disclosure include, but are not limited to, a nucleic acid sequence, an antibody ligand, biotin, avidin, streptavidin, digoxigenin, a magnetic bead (e.g., DYNABEADS® by ThermoFisher, encompassing functionalized magnetic beads such as DYNABEADS® M-270 amine by ThermoFisher). In aspects, the affinity agent is a nucleic acid sequence. In aspects, the affinity agent is an antibody ligand. In aspects, the affinity agent is a biotin. In aspects, the affinity agent is avidin. In aspects, the affinity agent is a streptavidin. In aspects, the affinity agent is a digoxigenin. In aspects, the affinity agent is a magnetic bead. In aspects, the affinity agent is a DYNABEADS® magnetic bead. In aspects, the affinity agent is a carbohydrate. In aspects, the affinity agent is a heparin. In aspects, the affinity agent is a dye ligand. In aspects, the affinity agent is an immunoaffinity agent. In aspects, the affinity agent is a monoclonal antibody. In aspects, the affinity agent is a lectin. In aspects, the affinity agent is a fusion protein. In aspects, the affinity agent is a boronic acid or a boronate. In aspects, the affinity agent is linked to a fluorophore (e.g., biotin linked to a fluorophore; avid linked to a fluorophore; streptavidin linked to a fluorophore).

“Fluorophore” refers to compounds that absorb light energy of a specific wavelength and re-emit the light at a lower wavelength. Exemplary fluorophores that may be used herein include xanthenes (e.g., fluorescein, rhodamine, Oregon green, eosin, Texas red); cyanines (e.g., cyanine, indocarbocyanine, oxacarbocyanine, thiacarbocyanine, merocyanine); squaraines (e.g., Seta, Square dyes); squaraine rotaxane (e.g., SeTau® dyes); naphthalenes (e.g., dansyl, prodan); coumarins; oxadiazoles (e.g., pyridyloxazole, nitrobenzoxadiazole, benzooxadiazole); anthracenes (e.g., anthraquinones, DRAQ5@, DRAQ7@, CyTRAK® orange); pyrenes (e.g., cascade blue); oxazines (e.g., Nile red, Nile blue, cresyl violet, oxazine 170); acridines (e.g., proflavin, acridine organge, acridine yellow); arylmethines (e.g., auramine, crystal violet, malachite green); tetrapyrroles (e.g., porphin, phthalocyanine, bilirubin), and the like. In embodiments, “fluorophore” is a fluorophore bound to avidin (e.g., Alexa Fluor® Avidin by ThermoFisher; or Rhodamine Avidin, Fluorescein Avidin, Texas Red® Aavidin all by Vector Laboratories). In embodiments, “fluorophore” is a fluorophore bound to streptavidin (e.g., Alexa Fluor® Streptavidin by ThermoFisher; or DyLight Streptavidin, Cy3 Streptavidin, Fluorescein Streptavidin, Texas Red® Streptavidin all by Vector Laboratories).

Radioactive substances (e.g., radioisotopes) that may be used as imaging and/or labeling agents in accordance with the embodiments of the disclosure include, but are not limited to, ¹⁸F, ³²p, ³³P, ⁴⁵Ti, ⁴⁷Sc, ⁵²Fe, ⁵⁹Fe, ⁶²Cu, ⁶⁴Cu, ⁶⁷Cu, ⁶⁷Ga ⁶⁸Ga⁷⁷As, ⁸⁶Y ⁹⁰Y. ⁸⁹Sr, ⁸⁹Zr, ⁹⁴Tc, ⁹⁴Tc, ^{99m}Tc, 99Mo, ¹⁰⁵Pd, ¹⁰⁵Rh, ¹¹¹Ag, ¹¹¹In, ¹²³I, ¹²⁴I, ¹²⁵I, ¹³¹I, ¹⁴²Pr, ¹⁴³Pr, ¹⁴⁹Pm, ¹⁵³Sm, ¹⁵⁴⁻¹⁵⁸¹Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴Ir, ¹⁹⁸Au, ¹⁹⁹Au, ²¹¹At, ²¹¹Pb, ²¹²Bi, ²¹²Pb, ²¹³Bi, ²²³Ra and ²²⁵Ac. Paramagnetic ions that may be used as additional imaging agents in accordance with the embodiments of the disclosure include, but are not limited to, ions of transition and lanthanide metals (e.g., metals having atomic numbers of 21-29, 42, 43, 44, or 57-71). These metals include ions of Cr, V, Mn, Fe, Co, Ni, Cu, La, Ce, Pr, Nd, Pm, Sm, Eu, Gd, Tb, Dy, Ho, Er, Tm, Yb and Lu.

The term “solid support” refers to an inert material or molecule (e.g., X¹) to which the compounds (haptens) herein may be immobilized directly or via a linking group (e.g., L¹). Solid supports are well-known in the art and are commercially available. In aspects, a solid support is a solid phase support including, e.g., resin beads, glass beads, silica chips, capillaries, dextran, crosslinked dextran gel (SEPHADEX®, by GE Healthcare Bioprocess R&D AB), crosslinked agarose (SEPHAROSE®, by GE Healthcare Bioprocess R&D AB), carboxymethyl cellulose polystyrene, ion-exchange resin, amino acid copolymer, or agarose. In aspects, a solid support is in the shape of, e.g., a tube, bead, disc, sphere, column, and the like.

“Carrier” refers to compounds or compositions that improve the therapeutic index of a drug by modifying drug absorption, reducing metabolism, prolonging biological half-life, or reducing toxicity. In embodiments, drug distribution is controlled primarily by properties of the carrier and no longer by physico-chemical characteristics of the drug substance only. Exemplary carriers that can be used herein include nanoparticles, liposomes, micelles, microspheres, virus-like particles, extracellular vesicles, synthetic peptide carriers, and the like.

Descriptions of compounds of the present disclosure are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

The term “leaving group” is used in accordance with its ordinary meaning in chemistry and refers to a moiety (e.g., atom, functional group, molecule) that separates from the molecule following a chemical reaction (e.g., bond formation, reductive elimination, condensation, cross-coupling reaction) involving an atom or chemical moiety to which the leaving group is attached, also referred to herein as the “leaving group reactive moiety,” and a complementary reactive moiety (i.e. a chemical moiety that reacts with the leaving group reactive moiety) to form a new bond between the remnants of the leaving groups reactive moiety and the complementary reactive moiety. Thus, the leaving group reactive moiety and the complementary reactive moiety form a complementary reactive group pair. Non limiting examples of leaving groups include hydrogen, hydroxide, organotin moieties (e.g., organotin heteroalkyl), halogen (e.g., Br), perfluoroalkylsulfonates (e.g. triflate), tosylates, mesylates, water, alcohols, nitrate, phosphate, thioether, amines, ammonia, fluoride, carboxylate, phenoxides, boronic acid, boronate esters, and alkoxides. In aspects, two molecules with leaving groups are allowed to contact, and upon a reaction and/or bond formation (e.g., acyloin condensation, aldol condensation, Claisen condensation, Stille reaction) the leaving groups separates from the respective molecule. In aspects, a leaving group is a bioconjugate reactive moiety. In aspects, at least two leaving groups (e.g., R¹ and R¹³) are allowed to contact such that the leaving groups are sufficiently proximal to react, interact or physically touch. In aspects, the leaving groups is designed to facilitate the reaction.

The term “protecting group” is used in accordance with its ordinary meaning in organic chemistry and refers to a moiety covalently bound to a heteroatom, heterocycloalkyl, or heteroaryl to prevent reactivity of the heteroatom, heterocycloalkyl, or heteroaryl during one or more chemical reactions performed prior to removal of the protecting group. Typically a protecting group is bound to a heteroatom (e.g., O) during a part of a multipart synthesis wherein it is not desired to have the heteroatom react (e.g., a chemical reduction) with the reagent. Following protection the protecting group may be removed (e.g., by modulating the pH). In aspects the protecting group is an alcohol protecting group. Non-limiting examples of alcohol protecting groups include acetyl, benzoyl, benzyl, methoxymethyl ether (MOM), tetrahydropyranyl (THP), and silyl ether (e.g., trimethylsilyl (TMS)). In aspects the protecting group is an amine protecting group. Non-limiting examples of amine protecting groups include carbobenzyloxy (Cbz), tert-butyloxycarbonyl (BOC), 9-Fluorenylmethyloxycarbonyl (FMOC), acetyl, benzoyl, benzyl, carbamate, p-methoxybenzyl ether (PMB), and tosyl (Ts).

A person of ordinary skill in the art will understand when a variable (e.g., moiety or linker) of a compound or of a compound genus (e.g., a genus described herein) is described by a name or formula of a standalone compound with all valencies filled, the unfilled valence(s) of the variable will be dictated by the context in which the variable is used. For example, when a variable of a compound as described herein is connected (e.g., bonded) to the remainder of the compound through a single bond, that variable is understood to represent a monovalent form (i.e., capable of forming a single bond due to an unfilled valence) of a standalone compound (e.g., if the variable is named “methane” in an embodiment but the variable is known to be attached by a single bond to the remainder of the compound, a person of ordinary skill in the art would understand that the variable is actually a monovalent form of methane, i.e., methyl or —CH₃). Likewise, for a linker variable (e.g., L¹ as described herein), a person of ordinary skill in the art will understand that the variable is the divalent form of a standalone compound (e.g., if the variable is assigned to “PEG” or “polyethylene glycol” in an embodiment but the variable is connected by two separate bonds to the remainder of the compound, a person of ordinary skill in the art would understand that the variable is a divalent (i.e., capable of forming two bonds through two unfilled valences) form of PEG instead of the standalone compound PEG).

The term “pharmaceutically acceptable salts” is meant to include salts of active compounds that are prepared with relatively nontoxic acids or bases, depending on the particular substituents found on the compounds described herein. When compounds disclosed herein contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds disclosed herein contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, oxalic, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science, 66:1-19 (1977)).

The terms “treating”, or “treatment” refer to any indicia of success in the treatment or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; or improving a patient's physical or mental well-being. The treatment or amelioration of symptoms can be based on objective or subjective parameters, including the results of a physical examination, neuropsychiatric exams, and/or a psychiatric evaluation. The term “treating” and conjugations thereof, include prevention of an injury, pathology, condition, or disease.

An “effective amount” is an amount sufficient to accomplish a stated purpose (e.g., achieve the effect for which it is administered, treat a disease, reduce enzyme activity, increase enzyme activity, reduce one or more symptoms of a disease or condition). An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

For any compound described herein, the therapeutically effective amount can be initially determined from cell culture assays. Target concentrations will be those concentrations of active compound(s) that are capable of achieving the methods described herein, as measured using the methods described herein or known in the art. As is well known in the art, therapeutically effective amounts for use in humans can also be determined from animal models. For example, a dose for humans can be formulated to achieve a concentration that has been found to be effective in animals. The dosage in humans can be adjusted by monitoring compounds effectiveness and adjusting the dosage upwards or downwards, as described above. Adjusting the dose to achieve maximal efficacy in humans based on the methods described above and other methods is well within the capabilities of the ordinarily skilled artisan.

Dosages may be varied depending upon the requirements of the patient and the compound being employed. The dose administered to a patient, in the context of the methods disclosed herein should be sufficient to effect a beneficial therapeutic response in the patient over time. The size of the dose also will be determined by the existence, nature, and extent of any adverse side-effects. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. Dosage amounts and intervals can be adjusted individually to provide levels of the administered compound effective for the particular clinical indication being treated. This will provide a therapeutic regimen that is commensurate with the severity of the individual's disease state.

Utilizing the teachings provided herein, an effective prophylactic or therapeutic treatment regimen can be planned that does not cause substantial toxicity and yet is effective to treat the clinical symptoms demonstrated by the particular patient. This planning should involve the careful choice of active compound by considering factors such as compound potency, relative bioavailability, patient body weight, presence and severity of adverse side effects, preferred mode of administration and the toxicity profile of the selected agent.

“Selective” or “selectivity” or the like of a compound refers to the compound's ability to discriminate between molecular targets or drugs. “Specific”, “specifically”, “specificity”, or the like of a compound refers to the compound's ability to cause a particular action, such as inhibition, to a particular target with minimal or no action to other targets.

“Pharmaceutically acceptable excipient” refer to a substance that aids the administration of an active agent to and absorption by a subject and can be included in the compositions and vaccines disclosed herein without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds disclosed herein. One of skill in the art will recognize that other pharmaceutical excipients are useful in the compositions, vaccines, and methods disclosed herein. Other exemplary excipients include macromolecules such as proteins, saccharides, polylactic acids, polyglycolic acids, polymeric amino acids, amino acid copolymers, sucrose, trehalose, lactose and lipid aggregates (such as oil droplets or liposomes). The compositions and vaccines may also contain diluents, such as water, saline, glycerol, etc. Additionally, auxiliary substances, such as wetting or emulsifying agents, pH buffering substances, and the like, may be present. Sterile pyrogen-free, phosphate buffered physiologic saline is a typical excipient. Other pharmaceutically acceptable excipients are described in Gennaro, 2000, Remington: The Science and Practice of Pharmacy, 20^{th} edition, ISBN:0683306472.

The term “vaccine” refers to a composition that can provide active acquired immunity to and/or therapeutic effect (e.g. treatment) of a particular drug, disease, or pathogen. A vaccine typically contains one or more agents that can induce an immune response in a subject against a drug, pathogen, disease, i.e. a target drug, pathogen, or disease. The immunogenic agent stimulates the body's immune system to recognize the agent as a threat or indication of the presence of the target pathogen or disease, thereby inducing immunological memory so that the immune system can more easily recognize and destroy and/or eliminate any of the drug or pathogen on subsequent exposure. Vaccines can be prophylactic (e.g. preventing or ameliorating the effects of a future drug overdose) or therapeutic (e.g., treating opioid use disorder in a subject). The administration of vaccines is referred to vaccination. In aspects, a vaccine can comprise a hapten or a hapten-conjugate.

The term “adjuvant” or “vaccine adjuvant” or “pharmaceutically acceptable adjuvant” refer to compounds used in a vaccine to enhance (e.g., increase, accelerate, prolong, and/or target) the specific immune response to the vaccine antigen/conjugate/hapten (e.g., the compounds described herein and embodiments and aspects thereof) in order to enhance the subject's immune response to the vaccine. Suitable adjuvants include aluminum salts; calcium salts; iron salts; zinc salts; acylated tyrosine; acylated sugars; cationically or anionically derivatized saccharides; polyphosphazenes; biodegradable microspheres; monophosphoryl lipid A (MPL); lipid A derivatives; 3-O-deacylated MPL; quil A; saponin; QS21; tocol; Freund's Incomplete Adjuvant (Difco Laboratories, Detroit, Mich.); Merck Adjuvant 65 (Merck and Company, Inc., Rahway, N.J.); AS-2 (Smith-Kline Beecham, Philadelphia, Pa.), toll like receptor agonists (e.g., CpG ODNs); bioadhesives; mucoadhesives; microparticles; liposomes; polyoxyethylene ether formulations; polyoxyethylene ester formulations; muramyl peptides; imidazoquinolone compounds (e.g. imiquamod and its homologues), and the like. Human immunomodulators suitable for use as adjuvants include cytokines such as interleukins (e.g. IL-I, IL-2, IL-4, IL-5, IL-6, IL-7, IL-12, etc); macrophage colony stimulating factor; tumor necrosis factor; granulocyte; macrophage colony stimulating factor; and the like. In aspects, the adjuvant comprises a toll-like receptor agonist. In aspects, the adjuvant comprises an aluminum salt. In aspects, the adjuvant comprises a toll-like receptor agonist and an aluminum salt. In aspects, the adjuvant comprises an aluminum salt and monophosphoryl lipid A. In aspects, the adjuvant comprises squalene. In aspects, the adjuvant comprises monophosphoryl lipid A and QS-21. In aspects, the adjuvant comprises a toll-like receptor agonist, an aluminum salt, monophosphoryl lipid A, or a combination of two or more thereof.

“Aluminum salts” refer to salts of aluminum that are useful as a vaccine adjuvant. Exemplary aluminum salts include aluminum sulfate, aluminum phosphate, aluminum hydroxyphosphate, aluminum hydroxide, and potassium aluminum sulfate. Generally an aluminum salt is used in a vaccine in an amount from about 0.01 mg/dose to about 1 mg/dose. In aspects, an aluminum salt is used in vaccine in an amount from about 0.1 mg/dose to about 0.8 mg/dose.

“Toll-like receptors” or “TLRs” refer to type I transmembrane receptors, evolutionarily conserved between insects and humans. Ten TLRs have been established (TLRs 1-10). Members of the TLR family have similar extracellular and intracellular domains; their extracellular domains have been shown to have leucine-rich repeating sequences, and their intracellular domains are similar to the intracellular region of the interleukin-1 receptor (IL-1R). TLR cells are expressed differentially among immune cells and other cells (including vascular epithelial cells, adipocytes, cardiac myocytes and intestinal epithelial cells). The intracellular domain of the TLRs can interact with the adaptor protein Myd88, which also posses the IL-1R domain in its cytoplasmic region, leading to NF-KB activation of cytokines; this Myd88 pathway is one way by which cytokine release is effected by TLR activation. The main expression of TLRs is in cell types such as antigen presenting cells (e.g. dendritic cells, macrophages etc).

“Toll-like receptor agonist” or “TLR agonist” refers to a compound which is capable of causing a signaling response through a TLR signaling pathway, either as a direct ligand or indirectly through generation of endogenous or exogenous ligand. In aspects, the toll-like receptor agonist is a toll-like receptor 2 agonist, toll-like receptor 3 agonist, toll-like receptor 4 agonist, toll-like receptor 5 agonist, toll-like receptor 7 agonist, toll-like receptor 8 agonist, toll-like receptor 9 agonist. In aspects, the toll-like receptor 9 agonist is a CpG oligodeoxynucleotide (ODN), which are well-known in the art and available from commercial suppliers, such as InvivoGen, Enzo Life Sciences, Inc., and Integrated DNA Technologies.

A “CpG oligodeoxynucleotide” or “CpG ODN” is an oligodeoxynucleotide including a CpG motif, wherein the pyrimidine ring of the cytosine is unmethylated. Generally, CpG ODNs range from about 8 to 30 bases in size. CpG ODNs can stimulate an immune response. Unmethylated CpG motifs are recognized by the Toll-like receptor 9 (TLR9) expressed on immune cells (such as B cells, macrophages, and dendritic cells). The CpG DNA is taken up by an endocytic/phagocytic pathway. It is known that the interaction of CpG ODN with TLR9 triggers recruitment of a MyD 88 adaptor molecule, activation of an IL-IR kinase-1 and other factors, resulting in the production of cytokines.

The term “CpG motif” refers to a 5′ C nucleotide connected to a 3′ G nucleotide through a phosphodiester internucleotide linkage or a phosphodiester derivative internucleotide linkage. In aspects, a CpG motif includes a phosphodiester internucleotide linkage.

“Class A CpG ODN” or “A-class CpG ODN” or “D-type CpG ODN” or “Class A CpG DNA sequence” is used in accordance with its common meaning in the biological and chemical sciences and refers to a CpG motif including oligodeoxynucleotide including one or more of poly-G sequence at the 5′, 3′, or both ends; an internal palindrome sequence including CpG motif, or one or more phosphodiester derivatives (phosphorothioate) linking deoxynucleotides. In aspects, a Class A CpG ODN includes poly-G sequence at the 5′, 3′, or both ends; an internal palindrome sequence including CpG motif, and one or more phosphodiester derivatives linking deoxynucleotides. Examples of Class A CpG ODNs include ODN D19, ODN 1585, ODN 2216, and ODN 2336.

“Class B CpG ODN” or “B-class CpG ODN” or “K-type CpG ODN” or “Class B CpG DNA sequence” is used in accordance with its common meaning in the biological and chemical sciences and refers to a CpG motif including oligodeoxynucleotide including one or more of a 6mer motif including a CpG motif, phosphodiester derivatives linking all deoxynucleotides. In aspects, a Class B CpG ODN includes one or more copies of a 6mer motif including a CpG motif and phosphodiester derivatives (phosphorothioate) linking all deoxynucleotides. In aspects, a Class B CpG ODN includes one 6mer motif including a CpG motif. In aspects, a Class B CpG ODN includes two copies of a 6mer motif including a CpG motif. In aspects, a Class B CpG ODN includes three copies of a 6mer motif including a CpG motif. In aspects, a Class B CpG ODN includes four copies of a 6mer motif including a CpG motif Examples of Class B CpG ODNs include ODN 1668, ODN 1826, ODN 2006, and ODN 2007.

“Class C CpG ODN” or “C-class CpG ODN”” or “C-type CpG DNA sequence” is used in accordance with its common meaning in the biological and chemical sciences and refers to an oligodeoxynucleotide including a palindrome sequence including a CpG motif and phosphodiester derivatives (phosphorothioate) linking all deoxynucleotides. Examples of Class C CpG ODNs include ODN 2395 and ODN M362.

As may be used herein, the terms “nucleic acid,” “nucleic acid molecule,” “nucleic acid oligomer,” “oligonucleotide,” “nucleic acid sequence,” “nucleic acid fragment” and “polynucleotide” are used interchangeably and are intended to include, but are not limited to, a polymeric form of nucleotides covalently linked together that may have various lengths, either deoxyribonucleotides or ribonucleotides, or analogs, derivatives or modifications thereof. Different polynucleotides may have different three-dimensional structures, and may perform various functions, known or unknown. Non-limiting examples of polynucleotides include a gene, a gene fragment, an exon, an intron, intergenic DNA (including, without limitation, heterochromatic DNA), messenger RNA (mRNA), transfer RNA, ribosomal RNA, a ribozyme, cDNA, a recombinant polynucleotide, a branched polynucleotide, a plasmid, a vector, isolated DNA of a sequence, isolated RNA of a sequence, a nucleic acid probe, and a primer. Polynucleotides useful in the methods of the disclosure may comprise natural nucleic acid sequences and variants thereof, artificial nucleic acid sequences, or a combination of such sequences.

With reference to the Diagnostic and Statistical Manual for Mental Disorders, 5th Edition, American Psychiatric Association, 2013 (also referred to herein as DSM5), the disclosure of which is incorporated by reference herein in its entirety, “opioid use disorder” is characterized by signs and symptoms that reflect compulsive, prolonged self-administration of opioid substances that are used for no legitimate medical purpose or, if another medical condition is present that requires opioid treatment, they are used in doses greatly in excess of the amount needed for that medical condition. In aspects, the opioid use disorder is moderate opioid use disorder. “Moderate opioid use disorder” is defined by reference to the DSM5 Opioid Use Disorder Checklist (ICD-9-CM code 304.00 or ICD-10-CM code F11.20) as having the presence of 4 or 5 symptoms indicated in the DSM5 Opioid Use Disorder Checklist. In aspects, the opioid use disorder is severe opioid use disorder. “Severe opioid use disorder” is defined by reference to the DSM5 Opioid Use Disorder Checklist (ICD-9-CM code 304.00 or ICD-10-CM code F11.20) as having the presence of 6 or more symptoms indicated in the DSM5 Opioid Use Disorder Checklist. In aspects, the opioid use disorder is moderate-to-severe opioid use disorder. Moderate-to-severe opioid use disorder refers to the presence of 4 or more symptoms indicated in the DSM5 Opioid Use Disorder Checklist. In aspects, the opioid use disorder is mild opioid use disorder. “Mild opioid use disorder” is defined by reference to the DSM5 Opioid Use Disorder Checklist (ICD-9-CM code 305.50 or ICD-10-CM code F11.10) as having the presence of 2 or 3 symptoms indicated in the DSM5 Opioid Use Disorder Checklist. In aspects, the opioid use disorder is mild-to-moderate opioid use disorder. Mild-to-moderate opioid use disorder refers to the presence of 2 to 5 symptoms indicated in the DSM5 Opioid Use Disorder Checklist. In aspects, “treating opioid use disorder” encompasses one or more of: (i) reducing opioid withdrawal symptoms, (ii) eliminating opioid withdrawal symptoms, (iii) reducing opioid craving, (iv) eliminating opioid craving, (v) reducing illicit opioid use, (vi) eliminating illicit opioid use, and (vii) inducing opioid abstinence. The term “opioid use disorder” can be interchangeably used with the terms “opioid addiction” or “opioid dependence.”

As used herein, the term “administering” means oral administration, administration as a suppository, topical contact, intravenous, parenteral, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. In aspects, the term administering means vaccination.

The dosage and frequency (single or multiple doses) administered to a mammal can vary depending upon a variety of factors, for example, whether the mammal suffers from another disease, and its route of administration; size, age, sex, health, body weight, body mass index, and diet of the recipient; nature and extent of symptoms of the disease being treated, kind of concurrent treatment, complications from the disease being treated or other health-related problems. Other therapeutic regimens or agents can be used in conjunction with the methods and compounds disclosed herein. Adjustment and manipulation of established dosages (e.g., frequency and duration) are well within the ability of those skilled in the art.

The compounds described herein can be used in combination with one another, with other active drugs known to be useful in treating a disease (e.g., buprenorphine or methadone) or with adjunctive agents that may not be effective alone, but may contribute to the efficacy of the active agent. Thus, the compounds described herein may be co-administered with one another or with other active drugs known to be useful in treating a disease.

“Patient,” “subject,” “patient in need thereof,” and “subject in need thereof” are herein used interchangeably and refer to a living organism suffering from or prone to a disease or condition that can be treated by administration of a compound or vaccine as provided herein. Non-limiting examples include humans, other mammals, bovines, rats, mice, dogs, monkeys, goat, sheep, cows, and other non-mammalian animals. In aspects, a patient is human.

“Disease,” “disorder” or “condition” refer to a state of being or health status of a patient or subject capable of being treated with the compounds or methods provided herein.

Compounds and Conjugates

The compounds described herein may be referred to as haptens or hapten conjugates. A “hapten” refers to a compound that is capable of generating or inducing the production of antibodies (e.g., compounds of Formula (A), (A1), (D1), (D3) and embodiments and aspects thereof). A “hapten conjugate” refers to a compound that is capable of generating or inducing the production of antibodies greater than a hapten alone (i.e., when X¹ is a protein) or a compound that is a capable of functioning as a probe (i.e., when X¹ is an affinity moiety and/or a detectable moiety) (e.g., compounds of Formula (B), (B1)-(B7), (D2), (D4), (D5) and embodiments and aspects thereof).

The disclosure provides a compound of Formula (A):

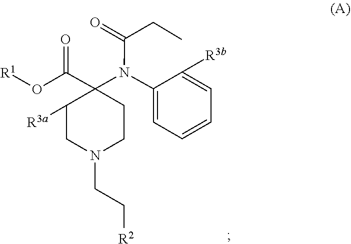

wherein R¹ is substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R² is substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; and R^{3a} and R^{3b} are each independently selected from the group consisting of hydrogen, C₁-C₆ alkyl, 2 to 6 membered heteroalkyl, oxo, halogen, —CCl₃, —CBr₃, —CF₃, —Cl₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, and —OCH₂F. In aspects, R^{3a} and R^{3b} are hydrogen. In aspects, R² is substituted or unsubstituted aryl, substituted or unsubstituted heteroaryl, substituted or unsubstituted heteroalkyl. In aspects, R² is unsubstituted aryl, substituted or unsubstituted heteroaryl, or substituted heteroalkyl. In aspects, R² is —C(O)—O—CH₃;

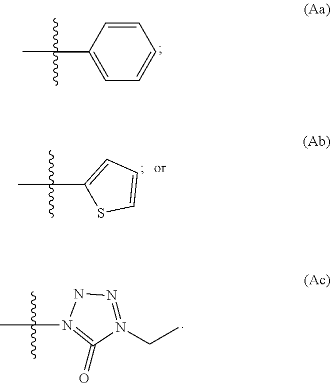

In aspects, R² is (Aa). In aspects, R² is (Ab). In aspects, R² is (Ac). In aspects, R² is —C(O)—O—CH₃. In aspects, R² is (Aa) and R^{3a} and R^{3b} are hydrogen. In aspects, R² is (Aa), R^{3a} is methyl, and R^{3b} is hydrogen. In aspects, R² is (Ab) and R^{3a} and R^{3b} are hydrogen. In aspects, R² is (Ac) and R^{3a} and R^{3b} are hydrogen. In aspects, R² is (Ac), R^{3a} is methyl, and R^{3b} is fluorine. In aspects, R² is (Ac), R^{3a} is hydrogen, and R^{3b} is fluorine. In aspects, R² is (Ac), R^{3a} is —CH₂—O—CH₃, and R^{3b} is hydrogen. In aspects, R² is —C(O)—O—CH₃ and R^{3a} and R^{3b} are hydrogen.

In embodiments, the compound of Formula (A) is a compound of Formula (A1):

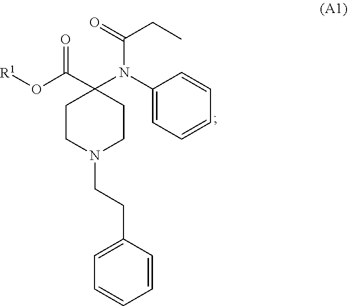

wherein R¹ is substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; with the proviso that R¹ is not methyl.

In embodiments, the compound of Formula (A) is a compound of Formula (A2):

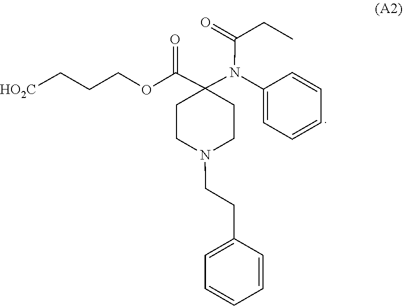 [SMILES: CCC(=O)N(c1ccccc1)C1(C(=O)OCCCC(=O)O)CCN(CCc2ccccc2)CC1]

The disclosure provides compounds of Formula (B):

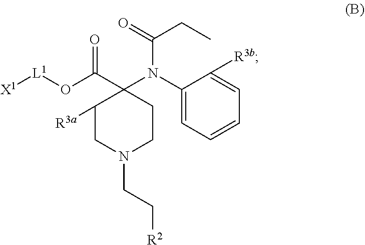

wherein X¹ comprises a protein, a detectable moiety, an affinity moiety, a carrier, a leaving group, or a protecting group; L¹ is a bond, —C(O)—, —C(O)O—, —O—, —S—, —NH—, —C(O)NH—, —NHC(O)—, —S(O)₂—, —S(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene; wherein R² is substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; and R^{3a} and R^{3b} are each independently selected from the group consisting of hydrogen, C₁-C₆ alkyl, 2 to 6 membered heteroalkyl, oxo, halogen, —CCl₃, —CBr₃, —CF₃, —Cl₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, and —OCH₂F. In aspects, R^{3a} and R^{3b} are hydrogen. In aspects, R² is substituted or unsubstituted aryl, substituted or unsubstituted heteroaryl, substituted or unsubstituted heteroalkyl. In aspects, R² is unsubstituted aryl, substituted or unsubstituted heteroaryl, or substituted heteroalkyl. In aspects, R² is —C(O)—O—CH₃;

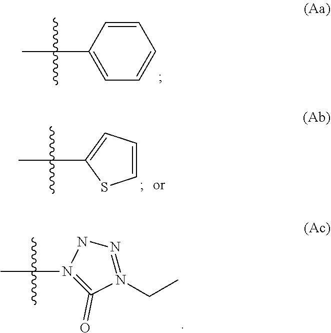

In aspects, R² is (Aa). In aspects, R² is (Ab). In aspects, R² is (Ac). In aspects, R² is —C(O)—O—CH₃. In aspects, R² is (Aa) and R^{3a} and R^{3b} are hydrogen. In aspects, R² is (Aa), R^{3a} is methyl, and R^{3b} is hydrogen. In aspects, R² is (Ab) and R^{3a} and R^{3b} are hydrogen. In aspects, R² is (Ac) and R^{3a} and R^{3b} are hydrogen. In aspects, R² is (Ac), R^{3a} is methyl, and R^{3b} is fluorine. In aspects, R² is (Ac), R^{3a} is hydrogen, and R^{3b} is fluorine. In aspects, R² is (Ac), R^{3a} is —CH₂—O—CH₃, and R^{3b} is hydrogen. In aspects, R² is —C(O)—O—CH₃ and R^{3a} and R^{3b} are hydrogen. In aspects, X¹ is a protein. In aspects, X¹ is a detectable moiety. In aspects, X¹ is an affinity moiety. In aspects, X¹ is a protecting group, a leaving group, a carrier, or a solid support. In aspects, X¹ comprises a detectable moiety and an affinity moiety (e.g., X¹ is streptavidin bound to a fluorophore; or avidin bound to a fluorophore).

In embodiments, the compound of Formula (B) is a compound of Formula (B1):

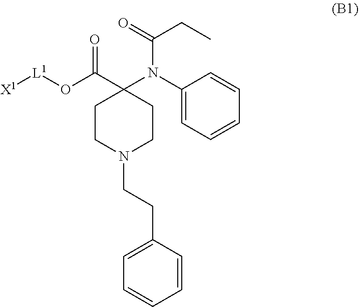

wherein X¹ comprises a protein, a detectable moiety, an affinity moiety, a solid support, a carrier, a leaving group, or a protecting group; and L¹ is a bond, —C(O)—, —C(O)O—, —O—, —S—, —NH—, —C(O)NH—, —NHC(O)—, —S(O)₂—, —S(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. In aspects, X¹ is a protein. In aspects, X¹ is a detectable moiety. In aspects, X¹ is an affinity moiety. In aspects, X¹ is a protecting group, a leaving group, a carrier, or a solid support. In aspects, X¹ comprises a detectable moiety and an affinity moiety (e.g., X¹ is streptavidin bound to a fluorophore; or avidin bound to a fluorophore).

In embodiments, the compound of Formula (B) is a compound of Formula (B2):

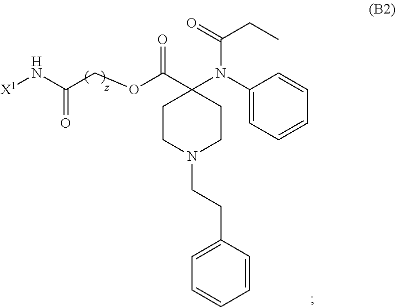

wherein z is an integer from 1 to 6, and X comprises a protein, a detectable moiety, an affinity moiety, a carrier, a leaving group, or a protecting group, as described in detail herein. In aspects, z is 1. In aspects, z is 2, In aspects, z is 3. In aspects, z is 4. In aspects, z is 5. In aspects, z is 6. In aspects, X¹ is a protein. In aspects, X¹ is a detectable moiety. In aspects, X¹ is an affinity moiety. In aspects, X¹ is a protecting group, a leaving group, a carrier, or a solid support. In aspects, X¹ comprises a detectable moiety and an affinity moiety (e.g., X¹ is streptavidin bound to a fluorophore; or avidin bound to a fluorophore).

In embodiments, the compound of Formula (B) is a compound of Formula (B3):

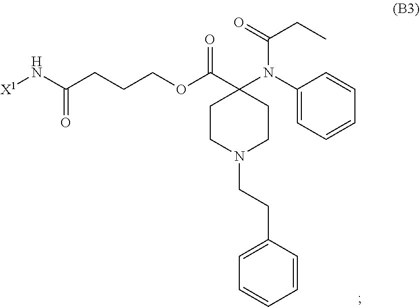

wherein X¹ comprises a protein, a detectable moiety, an affinity moiety, a solid support, a carrier, a leaving group, or a protecting group, as described in detail herein. In aspects, X¹ is a protein. In aspects, X¹ is a detectable moiety. In aspects, X¹ is an affinity moiety. In aspects, X¹ is a protecting group, a leaving group, a carrier, or a solid support. In aspects, X¹ comprises a detectable moiety and an affinity moiety (e.g., X¹ is streptavidin bound to a fluorophore; or avidin bound to a fluorophore).

In embodiments, the compound of Formula (B) is a compound of Formula (B4):

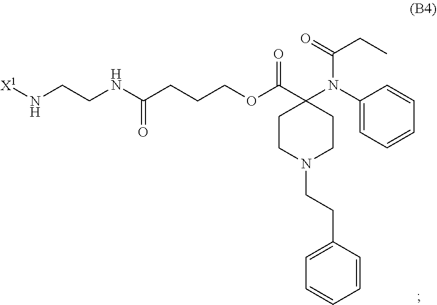

wherein X¹ comprises a protein, a detectable moiety, an affinity moiety, a carrier, a protecting group, or a leaving group, as described in detail herein. In aspects, X¹ is a protein. In aspects, X¹ is a detectable moiety. In aspects, X¹ is an affinity moiety. In aspects, X¹ is a protecting group, a leaving group, a carrier, or a solid support. In aspects, X¹ comprises a detectable moiety and an affinity moiety (e.g., X¹ is streptavidin bound to a fluorophore; or avidin bound to a fluorophore).

In embodiments, the compound of Formula (B) is a compound of Formula (B5):

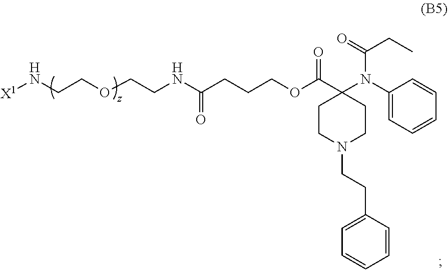

wherein z is an integer from 1 to 6; and X¹ comprises a protein, a detectable moiety, an affinity moiety, a solid support, a carrier, a leaving group, or a protecting group, as described in detail herein. In aspects, X¹ is a protein. In aspects, X¹ is a detectable moiety. In aspects, X¹ is an affinity moiety. In aspects, X¹ comprises a detectable moiety and an affinity moiety (e.g., X¹ is streptavidin bound to a fluorophore; or avidin bound to a fluorophore). In aspects, X¹ is a protecting group, a leaving group, a carrier, or a solid support. In aspects, z is 1. In aspects, z is 2, In aspects, z is 3. In aspects, z is 4. In aspects, z is 5. In aspects, z is 6.

In embodiments, the compound of Formula (B) is a compound of Formula (B6):

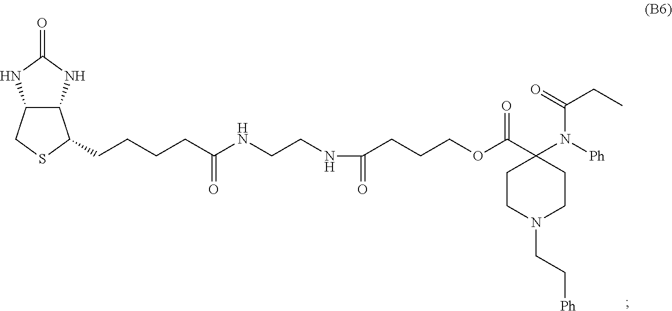 [SMILES: CCC(=O)N(c1ccccc1)C1(C(=O)OCCCC(=O)NCCNC(=O)CCCC[C@@H]2SC[C@@H]3NC(=O)N[C@@H]32)CCN(CCc2ccccc2)CC1]

wherein “Ph” is phenyl.

In embodiments, the compound of Formula (B) is a compound of Formula (B7):

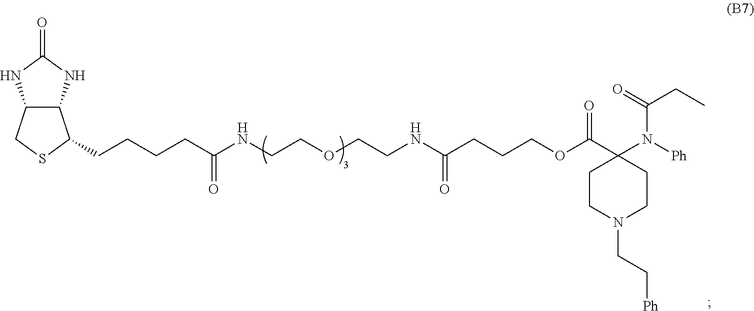

wherein “Ph” is phenyl.

In aspects, the disclosure provides a compound comprising the moiety of Formula (C1) and a detectable moiety. In aspects, the disclosure provides a compound comprising the moiety of Formula (C1) and a protein. In aspects, the disclosure provides a compound comprising the moiety of Formula (C2) and a protein. In aspects, the disclosure provides a compound comprising the moiety of Formula (C2) and a detectable moiety and/or affinity moiety. In aspects, the disclosure provides a compound comprising the moiety of Formula (C2) and a protein. The moiety of Formula (C1) and (C2) are:

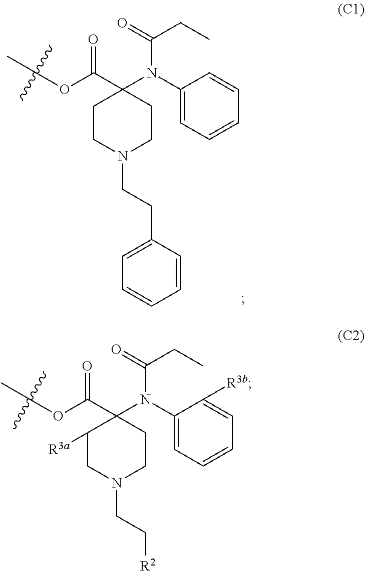

wherein R² is substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; and R^{3a} and R^{3b} are each independently selected from the group consisting of hydrogen, C₁-C₆ alkyl, 2 to 6 membered heteroalkyl, oxo, halogen, —CCl₃, —CBr₃, —CF₃, —Cl₃, CHCl₂, —CHBr₂, —CHF₂, —CHI₂, —CH₂C₁, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —OCHI₂, —OCHF₂, —OCH₂C₁, —OCH₂Br, —OCH₂I, and —OCH₂F. In aspects, R² is substituted or unsubstituted aryl, substituted or unsubstituted heteroaryl, substituted or unsubstituted heteroalkyl. In aspects, R² is unsubstituted aryl, substituted or unsubstituted heteroaryl, or substituted heteroalkyl. In aspects, R² is —C(O)—O—CH₃;

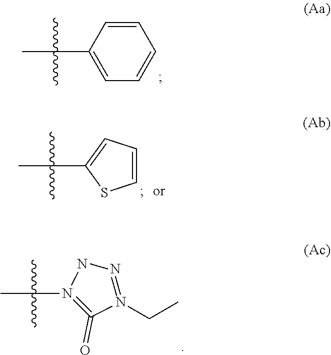

In aspects, R² is (Aa). In aspects, R² is (Ab). In aspects, R² is (Ac). In aspects, R² is —C(O)—O—CH₃. In aspects, R² is (Aa) and R^{3a} and R^{3b} are hydrogen. In aspects, R² is (Aa), R^{3a} is methyl, and R^{3b} is hydrogen. In aspects, R² is (Ab) and R^{3a} and R^{3b} are hydrogen. In aspects, R² is (Ac) and R^{3a} and R^{3b} are hydrogen. In aspects, R² is (Ac), R^{3a} is methyl, and R^{3b} is fluorine. In aspects, R² is (Ac), R^{3a} is hydrogen, and R^{3b} is fluorine. In aspects, R² is (Ac), R^{3a} is —CH₂—O—CH₃, and R^{3b} is hydrogen. In aspects, R² is —C(O)—O—CH₃ and R^{3a} and R^{3b} are hydrogen.

In embodiments, the disclosure provides a compound of Formula (D1) or (D2):

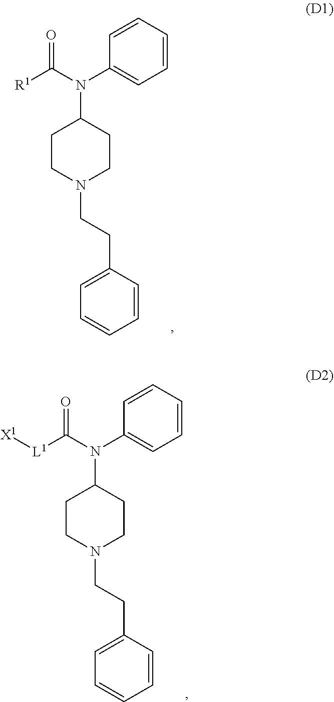

wherein R¹, L¹, and X¹ are as defined herein. In aspects, X¹ is a protein. In aspects, X¹ is a detectable moiety. In aspects, X¹ is an affinity moiety. In aspects, X¹ is a protecting group, a leaving group, a carrier, or a solid support. In aspects, X¹ comprises a detectable moiety and an affinity moiety (e.g., X¹ is streptavidin bound to a fluorophore; or avidin bound to a fluorophore).

In embodiments, the compound of Formula (D1) is a compound of Formula (D3):

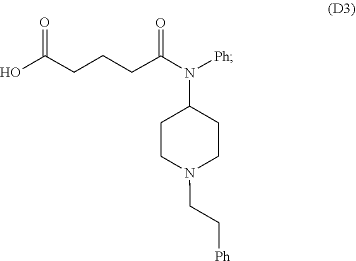 [SMILES: O=C(O)CCCC(=O)N(c1ccccc1)C1CCN(CCc2ccccc2)CC1]

wherein “Ph” is phenyl.

In embodiments, the compound of Formula (D2) is a compound of Formula (D4):

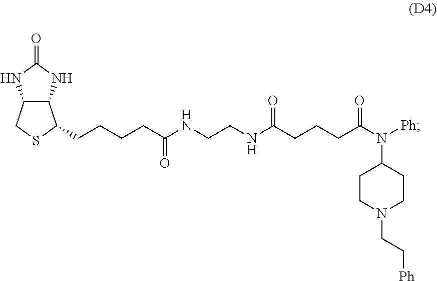 [SMILES: O=C(CCCC[C@@H]1SC[C@@H]2NC(=O)N[C@@H]21)NCCNC(=O)CCCC(=O)N(c1ccccc1)C1CCN(CCc2ccccc2)CC1]

wherein “Ph” is phenyl.

In embodiments, the compound of Formula (D2) is a compound of Formula (D5):

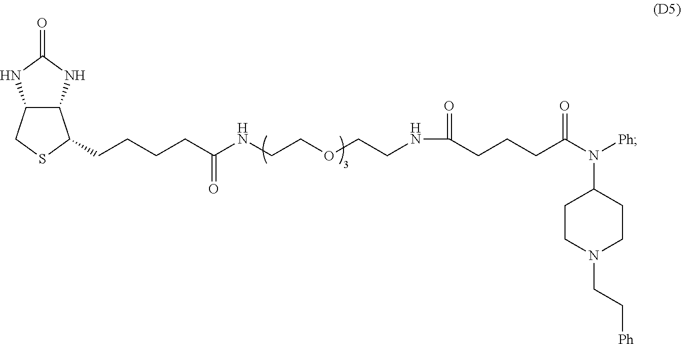

wherein “Ph” is phenyl.

In aspects, the disclosure provides a compound comprising the moiety of Formula (E) and a detectable moiety and/or an affinity moiety. In aspects, the disclosure provides a compound comprising the moiety of Formula (E) and a protein. The moiety of Formula (E) is:

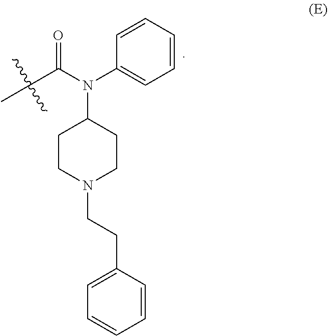 [SMILES: *C(=O)N(c1ccccc1)C1CCN(CCc2ccccc2)CC1]

Further Substituent Definitions for Compounds of the Disclosure

The following definitions for the substituents apply to each of the compounds described herein, including compounds of Formula (A), Formula (B), Formula (C), Formula (D), Formula (E), and embodiments and aspects of each of the foregoing.

In embodiments, R¹ is not methyl or ethyl. In aspects, R¹ is not an unsubstituted C₁-C₃ alkyl. In aspects, R¹ is not an unsubstituted C₁-C₄ alkyl. In aspects, R′ is not an unsubstituted C₁-C₆ alkyl. In aspects, R¹ is not an unsubstituted alkyl.

In embodiments, R¹ is unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R¹ is —CH₂(OH), R^{4A}-substituted or unsubstituted alkyl, R^{4A}-substituted or unsubstituted heteroalkyl, R^{4A}-substituted or unsubstituted cycloalkyl, R^{4A}-substituted or unsubstituted heterocycloalkyl, R^{4A}-substituted or unsubstituted aryl, or R^{4A}-substituted or unsubstituted heteroaryl; with the proviso that R¹ is not methyl or ethyl. In aspects, R¹ is unsubstituted alkyl, unsubstituted heteroalkyl, unsubstituted cycloalkyl, unsubstituted heterocycloalkyl, unsubstituted aryl, or unsubstituted heteroaryl; with the proviso that R¹ is not methyl or ethyl. In aspects, R¹ is R^{4A}-substituted alkyl, R^{4A}-substituted heteroalkyl, R^{4A}-substituted cycloalkyl, R^{4A}-heterocycloalkyl, R^{4A}-substituted aryl, or R^{4A}-substituted heteroaryl.

In embodiments, R^{4A} is halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCF₃, —OCHF₂, R^{4B}-substituted or unsubstituted alkyl, R^{4B}-substituted or unsubstituted heteroalkyl, R^{4B}-substituted or unsubstituted cycloalkyl, R^{4B}-substituted or unsubstituted heterocycloalkyl, R^{4B}-substituted or unsubstituted aryl, or R^{4B}-substituted or unsubstituted heteroaryl.

In embodiments, R^{4B} is halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, R^{4C}-substituted or unsubstituted alkyl, R^{4C}-substituted or unsubstituted heteroalkyl, R^{4C}-substituted or unsubstituted cycloalkyl, R^{4C}-substituted or unsubstituted heterocycloalkyl, R^{4C}-substituted or unsubstituted aryl, or R^{4C}-substituted or unsubstituted heteroaryl. R^{4C} is halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —OCF₃, —OCHF₂, unsubstituted alkyl, unsubstituted heteroalkyl, unsubstituted cycloalkyl, unsubstituted heterocycloalkyl, unsubstituted aryl, or unsubstituted heteroaryl.

In embodiments, R¹ is a size-limited substituent, wherein each substituted or unsubstituted alkyl is a substituted or unsubstituted C₁-C₂₀ alkyl (with the proviso that R¹ is not methyl or ethyl), each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, and each substituted or unsubstituted heterocycloalkyl is a substituted or unsubstituted 3 to 8 membered heterocycloalkyl. In aspects, R¹ is independently a lower substituent, wherein each substituted or unsubstituted alkyl is a substituted or unsubstituted C₁-C₈ alkyl (with the proviso that R¹ is not methyl or ethyl), each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, and each substituted or unsubstituted heterocycloalkyl is a substituted or unsubstituted 3 to 7 membered heterocycloalkyl.

In embodiments, one or more of R^{4A}, R^{4B}, and R^{4C} is a size-limited substituent, wherein each substituted or unsubstituted alkyl is independently a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, and each substituted or unsubstituted heterocycloalkyl is a substituted or unsubstituted 3 to 8 membered heterocycloalkyl. In aspects, R^{4A}, R^{4B}, and R^{4C} are each independently a lower substituent, wherein each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, and each substituted or unsubstituted heterocycloalkyl is a substituted or unsubstituted 3 to 7 membered heterocycloalkyl.

In embodiments, R¹ is —CH₂(OH). In aspects, R¹ is —C(O)OH. In aspects, R¹ is —NH₂. In aspects, R¹ is —C(O)NH₂. In aspects, R¹ is —NHCH(O). In aspects, R¹ is —NHCH₂(OH). In aspects, R¹ is —S(O)NH₂. In aspects, R¹ is substituted or unsubstituted 5-6 membered cycloalkyl. In aspects, R¹ is substituted or unsubstituted 5-6 membered heterocycloalkyl. In aspects, R¹ is substituted or unsubstituted 5-6 membered aryl. In aspects, R¹ is or substituted or unsubstituted 5-6 membered heteroaryl.

In embodiments, R¹ is substituted or unsubstituted 2 to 20 membered heteroalkyl. In aspects, R¹ is unsubstituted 2-18 membered heteroalkyl. In aspects, R¹ is substituted 2-18 membered heteroalkyl. In aspects, R¹ is substituted 2-16 membered heteroalkyl. In aspects, R¹ is substituted 2-12 membered heteroalkyl. In aspects, R¹ is substituted 2-10 membered heteroalkyl. In aspects, R¹ is substituted 2-8 membered heteroalkyl. The substituent for the heteroalkyl group can be any known in the art and described herein. In aspects, the heteroalkyl is substituted with 1, 2, 3, or 4 moieties selected from the group consisting of oxo, —CCl₃, —CBr₃, —CF₃, —Cl₃, —CHCl₂, —CHBr₂, —CHF₂, —CHI₂, —CH₂Cl, —CH₂Br, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, and —OCH₂F. In aspects, the heteroalkyl is substituted with 1, 2, or 3 moieties selected from the group consisting of oxo, —CN, —OH, —NH₂, —COOH, —CONH₂, —NO₂, —NHNH₂, —ONH₂, —NHC(O)NHNH₂, —NHC(O)NH₂, —NHC(O)H, —NHC(O)OH, and —NHOH. In aspects, the heteroalkyl is substituted with 1, 2, or 3 moieties selected from the group consisting of oxo, —CN, —OH, —NH₂, —COOH, —CONH₂, —NO₂, —NHNH₂, —ONH₂, —NHC(O)NHNH₂, —NHC(O)NH₂, —NHC(O)H, —NHC(O)OH, and —NHOH. In aspects, the heteroalkyl is substituted with 1, 2, or 3 moieties selected from the group consisting of oxo, —OH, —NH₂, —COOH, and —CONH₂. In aspects, the heteroalkyl contains 1 to 5 heteroatoms. In aspects, the heteroalkyl contains 1 to 4 heteroatoms. In aspects, the heteroalkyl contains 1 to 3 heteroatoms. In aspects, the heteroalkyl contains 1 or 2 heteroatoms.

In embodiments, R¹ is —(CH₂)_{z}—C(O)—NH—(CH₂)_{z}NH₂ or —(CH₂)_{z}—C(O)—NH—(CH₂)_{z}—CH₃, wherein z is an integer from 1 to 6. In aspects, R¹ is —(CH₂)₃—C(O)—NH—(CH₂)₂NH₂ or —(CH₂)₃—C(O)—NH—CH₂CH₃. In aspects, R¹ is —(CH₂)_{z}—C(O)OH or —(CH₂)_{z}—CO—NH₂, wherein z is an integer from 1 to 6. In aspects, R¹ is —(CH₂)₃—C(O)OH or —(CH₂)₃—CO—NH₂. In aspects, R¹ is —(CH₂)_{z}—C(O)—NH—(CH₂)_{z}—(OCH₂CH₂)_{z}NH—, wherein each z independently an integer from 1 to 6. In aspects each z is independently an integer from 1 to 4. In aspects, each z is independently an integer from 1 to 3. In aspects, R¹ is —(CH₂)₃—C(O)—NH—(CH₂)₂—(OCH₂CH₂)₃NH₂.

In embodiments, R¹ is substituted alkyl. In aspects, R¹ is substituted C₁-C₂₄ alkyl. In aspects, R¹ is substituted C₁-C₂₀ alkyl. In aspects, R¹ is unsubstituted C₁-C₁₆ alkyl. In aspects, R¹ is substituted C₁-C₁₂ alkyl. In aspects, R¹ is substituted C₁-C₁₀ alkyl. In aspects, R¹ is substituted C₁-C₈ alkyl. In aspects, R¹ is substituted C₁-C₆ alkyl. In aspects, R¹ is substituted C₁-C₄ alkyl. In aspects, R¹ is substituted C₂ alkyl. In aspects, R¹ is substituted C₃ alkyl. In aspects, R¹ is substituted C₄ alkyl. In aspects, R¹ is substituted C₅ alkyl. In aspects, R¹ is substituted C₆ alkyl. The substituent for the alkyl group can be any known in the art and described herein. In aspects, the alkyl group is substituted with one or more moieties selected from the group consisting of oxo, —CCl₃, —CBr₃, —CF₃, —Cl₃, —CHCl₂, —CHBr₂, —CHF₂, —CHI₂, —CH₂Cl, —CH₂Br, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, and —OCH₂F. In aspects, the alkyl is substituted with 1, 2, 3, or 4 moieties selected from the group consisting of oxo, —CN, —OH, —NH₂, —COOH, —CONH₂, —NO₂, —NHNH₂, —ONH₂, —NHC(O)NHNH₂, —NHC(O)NH₂, —NHC(O)H, —NHC(O)OH, and —NHOH. In aspects, the alkyl is substituted with is 1 or 2 moieties selected from the group consisting of oxo, —CN, —OH, —NH₂, —COOH, —CONH₂, —NO₂, —NHNH₂, —ONH₂, —NHC(O)NHNH₂, —NHC(O)NH₂, —NHC(O)H, —NHC(O)OH, and —NHOH. In aspects, the alkyl is substituted with is 1 moiety selected from the group consisting of oxo, —CN, —OH, —NH₂, —COOH, —CONH₂, —NO₂, —NHNH₂, —ONH₂, —NHC(O)NHNH₂, —NHC(O)NH₂, —NHC(O)H, —NHC(O)OH, and —NHOH. In aspects, the alkyl is substituted with is 1 or 2 moieties selected from the group consisting of oxo, —OH, —NH₂, —COOH, and —CONH₂. In aspects, the alkyl is substituted with is 1 moiety selected from the group consisting of oxo, —OH, —NH₂, —COOH, and —CONH₂.

In embodiments, R¹ is a substituted alkyl, wherein the alkyl is substituted with one or more —COOH moiety. In aspects, R¹ is a substituted C₁-C-₁₂ alkyl, wherein the alkyl is substituted with a —COOH moiety. In aspects, R¹ is a substituted C₁-C-₁₀ alkyl, wherein the alkyl is substituted with a —COOH moiety. In aspects, R¹ is a substituted C₁-C-₈ alkyl, wherein the alkyl is substituted with a —COOH moiety. In aspects, R¹ is —(CH₂)_{z}—COOH, wherein z is an integer from 1 to 6. In aspects, z is an integer from 2 to 4. In aspects, R¹ is —(CH₂)—COOH. In aspects, R¹ is —(CH₂)₂—COOH. In aspects, R¹ is —(CH₂)₄—COOH. In aspects, R¹ is —(CH₂)₅—COOH. In aspects, R¹ is —(CH₂)₆—COOH.

In embodiments, L¹ is a bond, —C(O)—, —C(O)O—, —O—, —S—, —NH—, —C(O)NH—, —NHC(O)—, —S(O)₂—, —S(O)NH—, R^{5A}-substituted or unsubstituted alkylene, R^{5A}-substituted or unsubstituted heteroalkylene, R^{5A}-substituted or unsubstituted cycloalkylene, R^{5A}-substituted or unsubstituted heterocycloalkylene, R^{5A}-substituted or unsubstituted arylene, or R^{5A}-substituted or unsubstituted heteroarylene. In aspects, L¹ is unsubstituted alkylene, unsubstituted heteroalkylene, unsubstituted cycloalkylene, unsubstituted heterocycloalkylene, unsubstituted arylene, or unsubstituted heteroarylene. In aspects, L¹ is R^{5A}-substituted alkylene, R^{5A}-substituted heteroalkylene, R^{5A}-substituted cycloalkylene, R^{5A}-heterocycloalkylene, R^{5A}-substituted arylene, or R^{5A}-substituted heteroarylene. R^{5A} is halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, R^{5B}-substituted or unsubstituted alkyl, R^{5B}-substituted or unsubstituted heteroalkyl, R^{5B}-substituted or unsubstituted cycloalkyl, R^{5B}-substituted or unsubstituted heterocycloalkyl, R^{5B}-substituted or unsubstituted aryl, or R^{5B}-substituted or unsubstituted heteroaryl.

In embodiments, R^{5B} is halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, R^{5C}-substituted or unsubstituted alkyl, R^{5C}-substituted or unsubstituted heteroalkyl, R^{5C}-substituted or unsubstituted cycloalkyl, R^{5C}-substituted or unsubstituted heterocycloalkyl, R^{5C}-substituted or unsubstituted aryl, or R^{5C}-substituted or unsubstituted heteroaryl. R^{5C} is halogen, —CF₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, unsubstituted alkyl, unsubstituted heteroalkyl, unsubstituted cycloalkyl, unsubstituted heterocycloalkyl, unsubstituted aryl, or unsubstituted heteroaryl.

In aspects, L¹ is a bond. In aspects, L¹ is —C(O)—. In aspects, L¹ is —C(O)O—. In aspects, L¹ is —O—. In aspects, L¹ is —S—. In aspects, L¹ is —NH—. In aspects, L¹ is —C(O)NH—. In aspects, L¹ is —NHC(O)—. In aspects, L¹ is —S(O)₂—. In aspects, L¹ is —S(O)NH—.

In embodiments, L¹ is substituted or unsubstituted alkylene. In aspects, L¹ is substituted alkylene. In aspects, L¹ is unsubstituted alkylene. In aspects, L¹ is substituted or unsubstituted C₂₋₂₄ alkylene. In aspects, L¹ is substituted or unsubstituted C₂₋₂₀ alkylene. In aspects, L¹ is substituted or unsubstituted C₂₋₁₈ alkylene. In aspects, L¹ is substituted or unsubstituted C₂₋₁₂ alkylene. In aspects, L¹ is substituted or unsubstituted C₂₋₆ alkylene. When the alkylene is substituted, the substituents may be any described herein or known in the art.

In aspects, the alkylene group is substituted with one or more moieties selected from the group consisting of oxo, halogen, —CCl₃, —CBr₃, —CF₃, —Cl₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂ NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, and —OCH₂F. In aspects, the alkylene group is substituted with 1, 2, 3, or 4 moieties selected from the group consisting of oxo, —CN, —OH, —NH₂, —COOH, —CONH₂, —NO₂, —NHNH₂, —ONH₂, —NHC(O)NHNH₂, —NHC(O)NH₂, —NHC(O)H, —NHC(O)OH, and —NHOH. In aspects, the alkylene group is substituted with 1 or 2 moieties selected from the group consisting of oxo, —CN, —OH, —NH₂, —COOH, —CONH₂, —NO₂, —NHNH₂, —ONH₂, —NHC(O)NHNH₂, —NHC(O)NH₂, —NHC(O)H, —NHC(O)OH, and —NHOH. In aspects, the alkylene group is substituted with 1, 2, or 3 moieties selected from the group consisting of oxo, —OH, —NH₂, —COOH, and —CONH₂. In aspects, the alkylene group is substituted with 1 or 2 moieties selected from the group consisting of oxo, —OH, —NH₂, —COOH, and —CONH₂.

In embodiments, L¹ is substituted or unsubstituted heteroalkylene. In aspects, L¹ is substituted heteroalkylene. In aspects, L¹ is unsubstituted heteroalkylene. In aspects, L¹ is substituted or unsubstituted cycloalkylene. In aspects, L¹ is substituted or unsubstituted heterocycloalkylene. In aspects, L¹ is substituted or unsubstituted arylene. In aspects, L¹ is substituted or unsubstituted heteroarylene.

In embodiments, L¹ is substituted or unsubstituted 2 to 24 membered heteroalkylene. In aspects, L¹ is substituted or unsubstituted 2 to 20 membered heteroalkylene. In aspects, L¹ is substituted or unsubstituted 2 to 18 membered heteroalkylene. In aspects, L¹ is substituted or unsubstituted 2 to 16 membered heteroalkylene. In aspects, L¹ is substituted or unsubstituted 2 to 14 membered heteroalkylene. In aspects, L¹ is substituted or unsubstituted 2 to 12 membered heteroalkylene. In aspects, L¹ is substituted or unsubstituted 2 to 10 membered heteroalkylene. In aspects, L¹ is substituted or unsubstituted 2 to 8 membered heteroalkylene. In aspects, L¹ is substituted or unsubstituted 2 to 6 membered heteroalkylene. In aspects, L¹ is substituted 2 to 24 membered heteroalkylene. In aspects, L¹ is substituted 2 to 20 membered heteroalkylene. In aspects, L¹ is substituted 2 to 18 membered heteroalkylene. In aspects, L¹ is substituted 2 to 16 membered heteroalkylene. In aspects, L¹ is substituted 2 to 14 membered heteroalkylene. In aspects, L¹ is substituted 2 to 12 membered heteroalkylene. In aspects, L¹ is substituted 2 to 10 membered heteroalkylene. In aspects, L¹ is substituted 2 to 10 membered heteroalkylene. In aspects, L¹ is substituted 2 to 8 membered heteroalkylene. In aspects, L¹ is substituted 2 to 6 membered heteroalkylene. In aspects, L¹ is substituted 2 to 4 membered heteroalkylene. In aspects, the heteroalkylene comprises from 1 to 6 heteroatoms. In aspects, the heteroalkylene comprises from 1 to 5 heteroatoms. In aspects, the heteroalkylene comprises from 1 to 4 heteroatoms. In aspects, the heteroalkylene comprises from 1 to 3 heteroatoms. In aspects, the heteroalkylene comprises 1 or 2 heteroatoms. In aspects, the heteroalkylene comprises 6 heteroatoms. In aspects, the heteroalkylene comprises 5 heteroatoms. In aspects, the heteroalkylene comprises 4 heteroatoms. In aspects, the heteroalkylene comprises 3 heteroatoms. In aspects, the heteroalkylene comprises 2 heteroatoms. In aspects, the heteroalkylene comprises 1 heteroatom. In aspects, the heteroalkylene comprises oxygen and nitrogen heteroatoms. In aspects, the heteroalkylene comprises one or more nitrogen heteroatoms. In aspects, the heteroalkylene comprises one or more nitrogen heteroatoms, but not oxygen heteroatoms. In aspects, the heteroalkylene is substituted with one or more moieties selected from the group consisting of oxo, halogen, —CCl₃, —CBr₃, —CF₃, —Cl₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, and —OCH₂F. In aspects, the heteroalkylene is substituted with 1 or 2 moieties selected from the group consisting of oxo, halogen, —CCl₃, —CBr₃, —CF₃, —Cl₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, and —OCH₂F. In aspects, the heteroalkylene is substituted with 1, 2, 3, or 4 moieties selected from the group consisting of oxo, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, and —NHOH. In aspects, the heteroalkylene is substituted with 1, 2, or 3 moieties selected from the group consisting of oxo, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, and —NHOH. In aspects, the heteroalkylene is substituted with 1 or 2 moieties selected from the group consisting of oxo, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, and —NHOH. In aspects, the heteroalkylene is substituted with 1, 2, 3, or 4 moieties selected from the group consisting of oxo, —CN, —OH, —NH₂, —COOH, —CONH₂, —NO₂, —NHNH₂, —ONH₂, —NHC(O)NHNH₂, —NHC(O)NH₂, —NHSO₂H, —NHC(O)H, —NHC(O)OH, and —NHOH. In aspects, the heteroalkylene is substituted with 1, 2, or 3 moieties selected from the group consisting of oxo, —CN, —OH, —NH₂, —COOH, —CONH₂, —NO₂, —NHNH₂, —ONH₂, —NHC(O)NHNH₂, —NHC(O)NH₂, —NHSO₂H, —NHC(O)H, —NHC(O)OH, and —NHOH. In aspects, the heteroalkylene is substituted with 1 or 2 moieties selected from the group consisting of oxo, —CN, —OH, —NH₂, —COOH, —CONH₂, —NO₂, —NHNH₂, —ONH₂, —NHC(O)NHNH₂, —NHC(O)NH₂, —NHSO₂H, —NHC(O)H, —NHC(O)OH, and —NHOH. In aspects, the heteroalkylene is substituted with 1, 2, or 3 moieties selected from the group consisting of oxo, —OH, —NH₂, —COOH, and —CONH₂. In aspects, the heteroalkylene is substituted with 1 or 2 moieties selected from the group consisting of oxo, —OH, —NH₂, —COOH, and —CONH₂.

In embodiments, L¹ is —(CH₂)_{z}—C(O)—NH—(CH₂)_{z}NH—, wherein each z is independently an integer from 1 to 6. In aspects, each z is independently an integer of 1 to 4. In aspects, each z is independently an integer of 2 or 3. In aspects, each z is 2. In aspects, each z is 3. In aspects, one z is 2 and one z is 3. In aspects, L¹ is —(CH₂)₃—C(O)—NH—(CH₂)₂NH—.

In embodiments, L¹ is —(CH₂)_{z}—C(O)—NH—(CH₂)_{z}—, wherein each z is independently an integer from 1 to 6. In aspects, each z is independently an integer of 1 to 4. In aspects, each z is independently an integer of 2 or 3. In aspects, each z is 2. In aspects, each z is 3. In aspects, one z is 2 and one z is 3. In aspects, L¹ is —(CH₂)₃—C(O)—NH—(CH₂)₂—.

In embodiments, L¹ is —(CH₂)_{z}—CO—NH—, where z is an integer from 1 to 6. In aspects, z is 1. In aspects, z is 2. In aspects, z is 3. In aspects, z is 4. In aspects, z is 5. In aspects, z is 6.

In embodiments, L¹ is —(CH₂)_{z}—C(O)—, where z is an integer from 1 to 6. In aspects, z is 1. In aspects, z is 2. In aspects, z is 3. In aspects, z is 4. In aspects, z is 5. In aspects, z is 6.

In embodiments, L¹ is —(CH₂)_{z}—C(O)—NH—(CH₂)_{z}—(OCH₂CH₂)_{z}NH—, where each z is independently an integer from 1 to 6. In aspects, each z is independently an integer of 1 to 4. In aspects, each z is independently an integer of 2 or 3. In aspects, each z is 2. In aspects, each z is 3. In aspects, two z are 2 and one z is 3. In aspects, one z is 2 and two z are 3. In aspects, L¹ is —(CH₂)₃—C(O)—NH—(CH₂)₂—(OCH₂CH₂)₃NH—.

In embodiments, L¹ is —(CH₂)_{z}—C(O)—NH—(CH₂)_{z}—(OCH₂CH₂)_{z}—, where each z is independently an integer from 1 to 6. In aspects, each z is independently an integer of 1 to 4. In aspects, each z is independently an integer of 2 or 3. In aspects, each z is 2. In aspects, each z is 3. In aspects, two z are 2 and one z is 3. In aspects, one z is 2 and two z are 3. In aspects, L¹ is —(CH₂)₃—C(O)—NH—(CH₂)₂—(OCH₂CH₂)₃—.

In embodiments, L¹ is a size-limited substituent, wherein each substituted or unsubstituted alkyl is a substituted or unsubstituted C₁-C₂₀ alkylene, each substituted or unsubstituted heteroalkylene is a substituted or unsubstituted 2 to 20 membered heteroalkylene, each substituted or unsubstituted cycloalkylene is a substituted or unsubstituted C₃-C₈ cycloalkylene, and each substituted or unsubstituted heterocycloalkyl is a substituted or unsubstituted 3 to 8 membered heterocycloalkyl. In aspects, L¹ is independently a lower substituent, wherein each substituted or unsubstituted alkylene is a substituted or unsubstituted C₁-C₈ alkylene, each substituted or unsubstituted heteroalkylene is a substituted or unsubstituted 2 to 8 membered heteroalkylene, each substituted or unsubstituted cycloalkylene is a substituted or unsubstituted C₃-C₇ cycloalkylene, and each substituted or unsubstituted heterocycloalkylene is a substituted or unsubstituted 3 to 7 membered heterocycloalkylene.

In embodiments, one or more of R^{5A}, R^{5B}, and R^{5C} is a size-limited substituent, wherein each substituted or unsubstituted alkyl is independently a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, and each substituted or unsubstituted heterocycloalkyl is a substituted or unsubstituted 3 to 8 membered heterocycloalkyl. In aspects, R^{5A}, R^{5B}, are R^{5C} each independently a lower substituent, wherein each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, and each substituted or unsubstituted heterocycloalkyl is a substituted or unsubstituted 3 to 7 membered heterocycloalkyl.

In embodiments, X¹ is covalently bonded to L¹ through an amine group thereby forming an —NH— connecting moiety. The term “—NH— connecting moiety” refers, in the usual and customary sense, to a second amine acting as a covalent linkage between substituents on the amine nitrogen. In aspects, X¹ includes an —NH— group that serves as the point of attachment to L¹. In aspects, L¹ includes an —NH— group that serves as the point of attachment to X¹.

In embodiments, X¹ is a protein. In aspects, the protein is albumin (e.g., serum albumin, human serum albumin, bovine serum albumin), CRM197, tetanus toxoid, diphtheria toxoid, cholera toxoid (e.g., cholera toxin subunit b), keyhole limpet hemocyanin, flagellin, gamma globulin (e.g., human gamma globulin, bovine gamma globulin), polylysine, chicken IgG, Pseudomonas aeruginosa exoprotein A, Group A streptococcal toxins, pneumolysin of Streptococcus pneumoniae, filamentous haemagglutinin (FHA), FHA fragments of Bordetella pertussis, pili of Neisseria gonorrhoeae, pili of Neisseria meningitidis, outer membrane proteins of Neisseria meningitidis, outer membrane proteins of Neisseria gonorrhoeae, CG1 peptidase of Streptococcus, or a surface protein of Moraxella catarrhalis. In aspects, the protein is tetanus toxoid. In aspects, the protein is diphtheria toxoid. In aspects, the protein is albumin. In aspects, the protein is serum albumin. In aspects, the protein is human serum albumin. In aspects, the protein is bovine serum albumin. In aspects, the protein is gamma globulin. In aspects, the protein is human gamma globulin. In aspects, the protein is bovine gamma globulin. In aspects, the protein is keyhole limpet hemocyanin. In aspects, the protein is Pseudomonas aeruginosa exoprotein A. In aspects, the protein is cholera toxin subunit b. In aspects, the protein is flagellin. In aspects, the protein is CRM197.

In embodiments, X¹ is a detectable moiety. The detectable moiety can be any described herein. In aspects, the detectable moiety is a fluorophore or a magnetic bead. In aspects, the detectable moiety is not a radioisotope or radionuclide. In aspects, the detectable moiety is a fluorophore. In aspects, the detectable moiety is a magnetic bead. “Magnetic beads” encompass functionalized magnetic beads that provide a linking group to covalently attached X¹ to L¹. In aspects, the magnetic beads are DYNABEADS® by ThermoFisher. The magnetic beads can be functionalized with an amine group (e.g., DYNABEADS® M-270 Amine by ThermoFisher); a toluene-sulfonyl group (e.g., DYNABEADS® M-450 Tosylactivated by ThermoFisher); a carboxylic acid group (e.g., DYNABEADS® M-270 Carboxylic Acid by ThermoFisher or DYNABEADS® MyOne™ Carboxylic Acid); a streptavidin moiety (e.g., DYNABEADS® M-270 Streptavidin by ThermoFisher); an epoxy group (e.g., DYNABEADS® M-450 Epoxy by ThermoFisher); and the like. In aspects, X¹ comprises a detectable moiety and an affinity moiety (e.g., X¹ is streptavidin bound to a fluorophore; or avidin bound to a fluorophore).

In embodiments, X¹ is an affinity moiety. The affinity moiety can be any described herein. In aspects, the affinity moiety is biotin, avidin, streptavidin, a nucleic acid sequence, an antibody ligand, digoxigenin, or a magnetic bead. In aspects, the detectable moiety is biotin. In aspects, X¹ is avidin. In aspects, X¹ is streptavidin. In aspects, X¹ is biotin-avidin complex. In aspects, X¹ is biotin-streptavidin complex. In aspects, the affinity moiety is an enzyme. In aspects, the affinity moiety is a magnetic bead. In aspects, the magnetic beads are DYNABEADS® by ThermoFisher. The magnetic beads can be functionalized with an amine group (e.g., DYNABEADS® M-270 Amine by ThermoFisher); a toluene-sulfonyl group (e.g., DYNABEADS® M-450 Tosylactivated by ThermoFisher); a carboxylic acid group (e.g., DYNABEADS® M-270 Carboxylic Acid by ThermoFisher or DYNABEADS® MyOne™ Carboxylic Acid); a streptavidin moiety (e.g., DYNABEADS® M-270 Streptavidin by ThermoFisher); an epoxy group (e.g., DYNABEADS® M-450 Epoxy by ThermoFisher); and the like. In aspects, X¹ comprises a detectable moiety and an affinity moiety (e.g., X¹ is streptavidin bound to a fluorophore; or avidin bound to a fluorophore).

In embodiments, X¹ is a carrier. In aspects, the carrier is a nanoparticle, liposome, micelle, microsphere, virus-like particle, extracellular vesicle, or synthetic peptide carrier. In aspects, the carrier is a nanoparticle, liposome, micelle, microsphere, or virus-like particle, extracellular vesicle. In aspects, the carrier is a nanoparticle, liposome, micelle, or microsphere.

In embodiments, X¹ is a solid support, a leaving group, or a protecting group. In aspects, X¹ is a leaving group. In aspects, X¹ is a protecting group. In aspects, X¹ is a solid support.

Compositions and Vaccines

The compositions described herein may also be referred to as vaccines when they are intended to be administered to a subject for the purpose of generating antibodies and/or treating or preventing a disease, such as opioid use disorder.

In aspects, the disclosure provides a composition comprising the compound of Formula (A) and a pharmaceutically acceptable excipient. In embodiments, the disclosure provides a composition comprising the compound of Formula (A) and an adjuvant. In aspects, the disclosure provides a composition comprising the compound of Formula (A), a pharmaceutically acceptable excipient, and an adjuvant. The compound of Formula (A) may be the compound of Formula (A1), (A2), or any embodiment or aspect of the compound of Formula (A). The compositions may comprise any pharmaceutically acceptable excipients and/or adjuvants known in the art, such as those described herein. The compositions described herein may also be referred to as vaccines when they are intended to be administered to a subject for the purpose of generating antibodies and/or treating a disease and/or preventing a disease (e.g., opioid use disorder) and/or preventing an opioid overdose. In aspects, the compositions may be referred to as vaccines when X¹ is a protein.

In aspects, the disclosure provides a composition comprising the compound of Formula (B) and a pharmaceutically acceptable excipient. In embodiments, the disclosure provides a composition comprising the compound of Formula (B) and an adjuvant. In aspects, the disclosure provides a composition comprising the compound of Formula (B), a pharmaceutically acceptable excipient, and an adjuvant. The compound of Formula (B) may be the compound of Formula (B1), (B2), (B3), (B4), (B5), (B6), (B7), or any embodiment or aspect of the compound of Formula (B). The compositions may comprise any pharmaceutically acceptable excipients and/or adjuvants known in the art, such as those described herein. The compositions described herein may also be referred to as vaccines when they are intended to be administered to a subject for the purpose of generating antibodies and/or treating a disease and/or preventing a disease (e.g., opioid use disorder) and/or preventing an opioid overdose. In aspects, the compositions may be referred to as vaccines when X¹ is a protein, and may be referred to as probes when X¹ is an affinity moiety and/or a detectable moiety.

In aspects, the disclosure provides a composition comprising a pharmaceutically acceptable excipient and a compound which comprises a moiety of Formula (C1), (C2), or (E). In aspects, the disclosure provides a composition comprising an adjuvant and a compound which comprises a moiety of Formula (C1), (C2), or (E). In aspects, the disclosure provides a composition comprising a pharmaceutically acceptable excipient, an adjuvant, and a compound which comprises a moiety of Formula (C1), (C2), or (E). The compositions may comprise any pharmaceutically acceptable excipients and/or adjuvants known in the art, such as those described herein. The compositions described herein may also be referred to as vaccines when they are intended to be administered to a subject for the purpose of generating antibodies and/or treating a disease and/or preventing a disease (e.g., opioid use disorder) and/or preventing an opioid overdose. In aspects, the compositions may be referred to as vaccines when X¹ is a protein.

In embodiments, the disclosure provides a composition comprising the compound of Formula (D1)-(D5) and a pharmaceutically acceptable excipient. In embodiments, the disclosure provides a composition comprising the compound of Formula (D1)-(D5) and an adjuvant. In aspects, the disclosure provides a composition comprising the compound of Formula (D1)-(D5), a pharmaceutically acceptable excipient, and an adjuvant. In aspects, the compound of Formula (D1)-(D5) is any embodiment or aspect described herein. The compositions may comprise any pharmaceutically acceptable excipients and/or adjuvants known in the art, such as those described herein. The compositions described herein may also be referred to as vaccines when they are intended to be administered to a subject for the purpose of generating antibodies and/or treating a disease and/or preventing a disease (e.g., opioid use disorder) and/or preventing an opioid overdose. In aspects, the compositions may be referred to as vaccines when X¹ is a protein.

In aspects, the compositions are vaccines comprising an adjuvant. In aspects, the compositions are vaccines comprising a pharmaceutically excipient and an adjuvant. In aspects, the adjuvant comprises an aluminum salt. In aspects, the aluminum salt is aluminum sulfate, aluminum phosphate, aluminum hydroxyphosphate, aluminum hydroxide, potassium aluminum sulfate, or a combination of two or more thereof. In aspects, the aluminum salt is aluminum sulfate. In aspects, the aluminum salt is aluminum phosphate. In aspects, the aluminum salt is aluminum hydroxyphosphate. In aspects, the aluminum salt is aluminum hydroxide. In aspects, the aluminum salt is potassium aluminum sulfate.

In aspects, the compositions are vaccines comprising an adjuvant. In aspects, the compositions are vaccines comprising a pharmaceutically excipient and an adjuvant. In aspects, the adjuvant comprises a toll-like receptor agonist. In aspects, the toll-like receptor is toll-like receptor 2 agonist, toll-like receptor 3 agonist, toll-like receptor 4 agonist, toll-like receptor 5 agonist, toll-like receptor 7 agonist, toll-like receptor 8 agonist, toll-like receptor 9 agonist, or a combination of two or more thereof. In aspects, the toll-like receptor agonist is toll-like receptor 3 agonist. In aspects, the toll-like receptor agonist is toll-like receptor 9 agonist. In aspects, the toll-like receptor 9 agonist is a CpG ODN. In aspects, the CpG ODN is a CpG-A ODN, a CpG-B ODN, a CpG-C ODN, or a combination of two or more thereof. In aspects, the CpG ODN is a CpG-A ODN. In aspects, the CpG ODN is a CpG-B ODN. In aspects, the CpG ODN is a CpG-C ODN. In aspects, the CpG ODN is CpG ODN 1585, CpG ODN 2216, CpG ODN 2336, CpG ODN 1668, CpG ODN 1826, CpG ODN 2006, CpG ODN 2007, CpG ODN BW006, CpG ODN D-SL01, CpG ODN 2395, CpG ODN M362, CpG ODN D-SL03, or a combination of two or more thereof. In aspects, the CpG ODN is CpG ODN 1585. In aspects, the CpG ODN is CpG ODN 2216. In aspects, the CpG ODN is CpG ODN 2336. In aspects, the CpG ODN is CpG ODN 1668. In aspects, the CpG ODN is CpG ODN 1826. In aspects, the CpG ODN is CpG ODN 2006. In aspects, the CpG ODN is CpG ODN 2007. In aspects, the CpG ODN is CpG ODN BW006. In aspects, the CpG ODN is CpG ODN D-SL01. In aspects, the CpG ODN is CpG ODN 2395. In aspects, the CpG ODN is CpG ODN M362. In aspects, the CpG ODN is CpG ODN D-SL03.

In aspects, the compositions are vaccines comprising an adjuvant. In aspects, the compositions are vaccines comprising a pharmaceutically excipient and an adjuvant. In aspects, the adjuvant comprises an aluminum salt and a toll-like receptor agonist. In aspects, the aluminum salt is aluminum sulfate, aluminum phosphate, aluminum hydroxyphosphate, aluminum hydroxide, potassium aluminum sulfate, or a combination of two or more thereof. In aspects, the aluminum salt is aluminum sulfate. In aspects, the aluminum salt is aluminum phosphate. In aspects, the aluminum salt is aluminum hydroxyphosphate. In aspects, the aluminum salt is aluminum hydroxide. In aspects, the aluminum salt is potassium aluminum sulfate. In aspects, the toll-like receptor is toll-like receptor 2 agonist, toll-like receptor 3 agonist, toll-like receptor 4 agonist, toll-like receptor 5 agonist, toll-like receptor 7 agonist, toll-like receptor 8 agonist, toll-like receptor 9 agonist, or a combination of two or more thereof. In aspects, the toll-like receptor agonist is toll-like receptor 3 agonist. In aspects, the toll-like receptor agonist is toll-like receptor 9 agonist. In aspects, the toll-like receptor 9 agonist is a CpG ODN. In aspects, the CpG ODN is a CpG-A ODN, a CpG-B ODN, a CpG-C ODN, or a combination of two or more thereof. In aspects, the CpG ODN is a CpG-A ODN. In aspects, the CpG ODN is a CpG-B ODN. In aspects, the CpG ODN is a CpG-C ODN. In aspects, the CpG ODN is CpG ODN 1585, CpG ODN 2216, CpG ODN 2336, CpG ODN 1668, CpG ODN 1826, CpG ODN 2006, CpG ODN 2007, CpG ODN BW006, CpG ODN D-SL01, CpG ODN 2395, CpG ODN M362, CpG ODN D-SL03, or a combination of two or more thereof. In aspects, the CpG ODN is CpG ODN 1585. In aspects, the CpG ODN is CpG ODN 2216. In aspects, the CpG ODN is CpG ODN 2336. In aspects, the CpG ODN is CpG ODN 1668. In aspects, the CpG ODN is CpG ODN 1826. In aspects, the CpG ODN is CpG ODN 2006. In aspects, the CpG ODN is CpG ODN 2007. In aspects, the CpG ODN is CpG ODN BW006. In aspects, the CpG ODN is CpG ODN D-SL01. In aspects, the CpG ODN is CpG ODN 2395. In aspects, the CpG ODN is CpG ODN M362. In aspects, the CpG ODN is CpG ODN D-SL03.

In aspects, the adjuvant comprises a surfactant (e.g., hexadecylamine, octadecylamine, lysolecithin, dimethyldioctadecylammonium bromide, N,N-dioctadecyl-N′,N-bis(2-hydroxy-ethylpropane diamine), methoxyhexadecyl glycerol, pluronic polyols); polyanions (e.g., pyran, dextran sulfate, poly IC, polyacrylic acid, Carbopol); peptides (e.g., muramyl dipeptide, aimethylglycine), tuftsin, oil emulsions, B peptide subunits of E. coli, or a combination of two or more thereof. In aspects, the adjuvant comprises a surfactant.

The vaccines and compositions may be lyophilized or in aqueous form, i.e., solutions or suspensions. Liquid formulations allow the compositions to be administered direct from their packaged form, without the need for reconstitution in an aqueous medium, and are thus ideal for injection. Compositions may be presented in vials, or they may be presented in ready filled syringes. The syringes may be supplied with or without needles. A syringe will include a single dose of the composition, whereas a vial may include a single dose or multiple doses (e.g. 2, 3, or 4 doses). In aspects, the dose is for a human and may be administered by injection.

Liquid vaccines are also suitable for reconstituting other vaccines from a lyophilized form. Where a vaccine is to be used for such extemporaneous reconstitution, the disclosure provides a kit, which may comprise two vials, or may comprise one ready-filled syringe and one vial, with the contents of the syringe being used to reconstitute the contents of the vial prior to injection. Vaccines may be packaged in unit dose form or in multiple dose form (e.g. 2, 3, or 4 doses). For multiple dose forms, vials can be pre-filled syringes. Effective dosage volumes can be routinely established, but a typical human dose of the composition has an injection volume of 0.25 to 1 mL.

In embodiments, vaccines have a pH of between 6.0 and 8.0, and may be buffered at this pH. Stable pH may be maintained by the use of a buffer, such as a phosphate buffer or a histidine buffer. The composition should be sterile and/or pyrogen free. The compositions and vaccines may be isotonic. Vaccines may include an antimicrobial, particularly when packaged in a multiple dose format. Other antimicrobials may be used, such as 2-phenoxyethanol or parabens (methyl, ethyl, propyl parabens). Preservative may be added exogenously and/or may be a component of the bulk haptens or hapten conjugates which are mixed to form the composition (e.g. present as a preservative in pertussis antigens). Vaccines may comprise detergent e.g. a Tween (polysorbate), such as Tween 80. Detergents are generally present at low levels, e.g. <0.01%. Vaccines may include sodium salts (e.g. sodium chloride) to give tonicity.

Methods of Treatment.

In aspects, the disclosure provides methods of treating opioid use disorder and/or preventing opioid use disorder and/or treating an opioid overdose in a patient in need thereof comprising administering to the patient a therapeutically effective amount of a compound of Formula (A); wherein the opioid is carfentanil or a carfentanil analogue. In aspects, the compound of Formula (A) is a compound of Formula (A1) or any embodiments or aspects thereof. In aspects, the compound of Formula (A) is a compound of Formula (A2). In aspects, the methods are for treating opioid use disorder. In aspects, the methods are for preventing opioid use disorder. In aspects, the methods are for preventing opioid overdose. In aspects, the methods are for treating opioid use disorder and preventing opioid overdose. In aspects, the methods are for preventing opioid use disorder and preventing an opioid overdose. In aspects, the opioid is carfentanil, sufentanil, remifentanil, alfentanil, lofentanil, brifentanil, trefentanil, or a combination of two or more thereof. In aspects, the opioid is carfentanil. In aspects, the opioid is sufentanil. In aspects, the opioid is remifentanil. In aspects, the opioid is alfentanil. In aspects, the opioid is lofentanil. In aspects, the opioid is brifentanil. In aspects, the opioid is trefentanil.

The disclosure provides methods of treating opioid use disorder and/or preventing opioid use disorder and/or treating an opioid overdose in a patient in need thereof comprising administering to the patient a therapeutically effective amount of a compound of Formula (B1), (B2), (B3), (B4), (B5), (B6), (B7), or an embodiment or aspect thereof, wherein the opioid is carfentanil or a carfentanil analogue. In aspects, the methods are for treating opioid use disorder. In aspects, the methods are for preventing opioid use disorder. In aspects, the methods are for preventing opioid overdose. In aspects, the methods are for treating opioid use disorder and preventing opioid overdose. In aspects, the methods are for preventing opioid use disorder and preventing an opioid overdose. In aspects, the opioid is carfentanil, sufentanil, remifentanil, alfentanil, lofentanil, brifentanil, trefentanil, or a combination of two or more thereof. In aspects, the opioid is carfentanil. In aspects, the opioid is sufentanil. In aspects, the opioid is remifentanil. In aspects, the opioid is alfentanil. In aspects, the opioid is lofentanil. In aspects, the opioid is brifentanil. In aspects, the opioid is trefentanil.

The disclosure provides methods of treating opioid use disorder and/or preventing opioid use disorder and/or treating an opioid overdose in a patient in need thereof comprising administering to the patient a therapeutically effective amount of a compound of Formula (D1), (D2), (D3), (D4), (D5), or an embodiment or aspect thereof; wherein the opioid is fentanyl or a fentanyl analogue. In aspects, the methods are for treating opioid use disorder. In aspects, the methods are for preventing opioid use disorder. In aspects, the methods are for preventing opioid overdose. In aspects, the methods are for treating opioid use disorder and preventing opioid overdose. In aspects, the methods are for preventing opioid use disorder and preventing an opioid overdose. In aspects, the opioid is fentanyl, acetylfentanyl, butyrfentanyl, para-tolylfentanyl, 3-methylfentanyl, or α-methylfentanyl, or a combination of two or more thereof.

Effective Dosages

Vaccines and pharmaceutical compositions include compositions wherein the active ingredient is contained in a therapeutically effective amount, i.e., in an amount effective to achieve its intended purpose. The actual amount effective for a particular application will depend, inter alia, on the condition being treated, as judged by a practitioner in the medical arts.

The dosage and frequency (single or multiple doses) of compound or vaccine administered can vary depending upon a variety of factors, including route of administration; size, age, sex, health, body weight, body mass index, and diet of the recipient; nature and extent of symptoms of the disease being treated; presence of other diseases or other health-related problems; kind of concurrent treatment; and complications from any disease or treatment regimen. Other therapeutic regimens or agents and/or psychological counseling can be used in conjunction with the methods and compounds described herein.

Dosages may be varied depending upon the requirements of the patient and the compound being employed. The dose administered to a patient should be sufficient to effect a beneficial therapeutic response in the patient over time. The size of the dose also will be determined by the existence, nature, and extent of any adverse side effects. Generally, treatment is initiated with smaller dosages, which are less than the optimum dose of the compound. Thereafter, the dosage is increased by small increments until the optimum effect under circumstances is reached. Dosage amounts and intervals can be adjusted individually to provide levels of the administered compound effective for the particular clinical indication being treated. This will provide a therapeutic regimen that is commensurate with the severity of the individual's disease state.

Utilizing the teachings provided herein, an effective prophylactic or therapeutic treatment regimen can be planned that does not cause substantial toxicity and yet is entirely effective to treat and/or prevent the clinical symptoms demonstrated by the particular patient. This planning should involve the choice of active compound by considering factors such as potency, bioavailability, patient body weight, presence and severity of adverse side effects, preferred mode of administration, and the toxicity profile of the selected agent.

### Embodiments P1-P8

Embodiment P1. A fentanyl hapten of formula 1

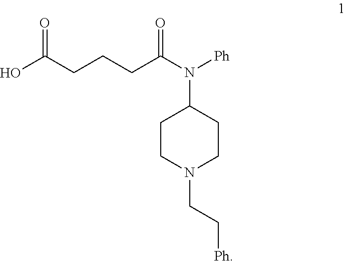 [SMILES: O=C(O)CCCC(=O)N(c1ccccc1)C1CCN(CCc2ccccc2)CC1]

Embodiment P2. A compound of formula 3

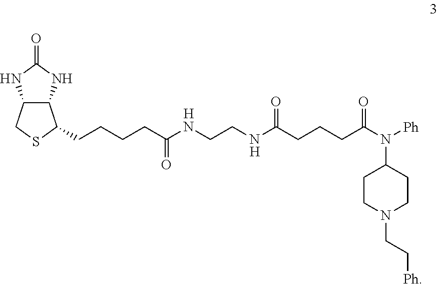

Embodiment P3. A compound of formula 5

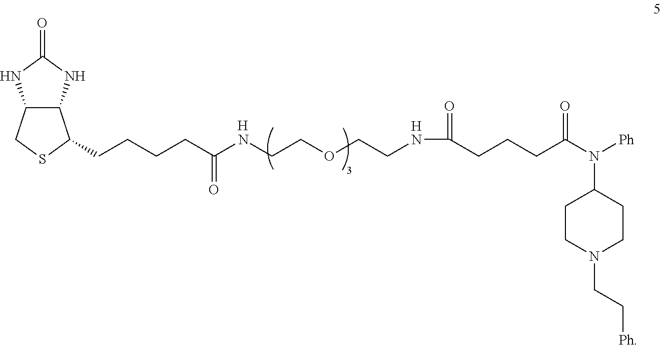

Embodiment P4. A compound of formula 10

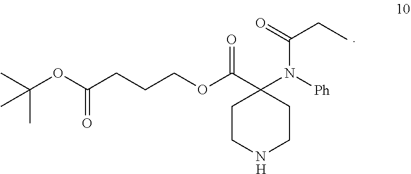

Embodiment P5. A carfentanil hapten of formula 12

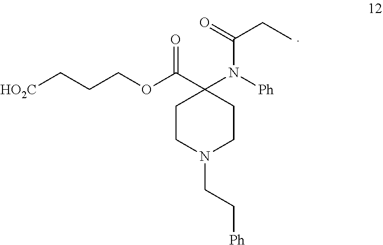 [SMILES: CCC(=O)N(c1ccccc1)C1(C(=O)OCCCC(=O)O)CCN(CCc2ccccc2)CC1]

Embodiment P6. A compound of formula 13

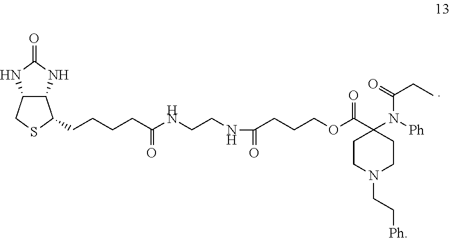

Embodiment P7. A compound of formula 14

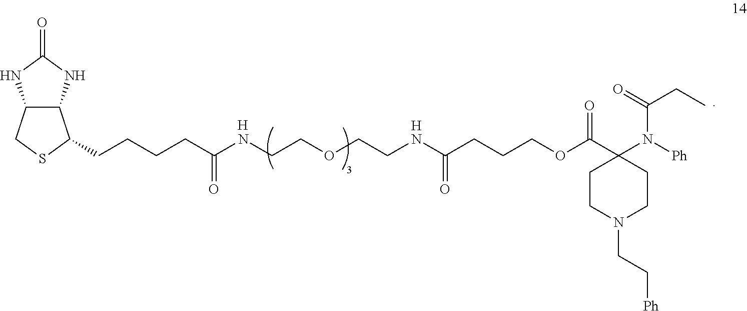

Embodiment P8. A method of isolating an antibody having high affinity for a fentanyl compound and a broad range of specificity among a set of fentanyl compounds, comprising immunizing a mammal with a hapten-carrier complex comprising the fentanyl hapten of formula 1 or the carfentanil hapten of formula 12:

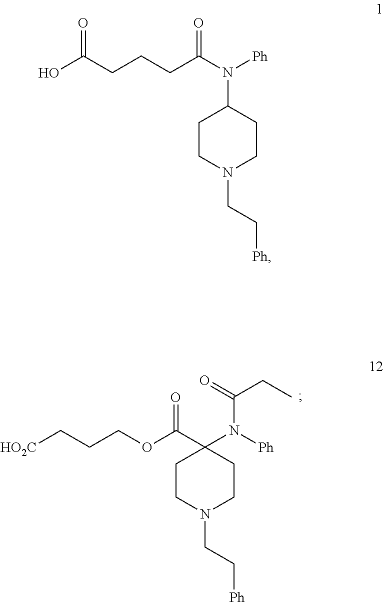 [SMILES: O=C(O)CCCC(=O)N(c1ccccc1)C1CCN(CCc2ccccc2)CC1 ; CCC(=O)N(c1ccccc1)C1(C(=O)OCCCC(=O)O)CCN(CCc2ccccc2)CC1]

then, screening the resulting set of antibodies using compound 13 or compound 14

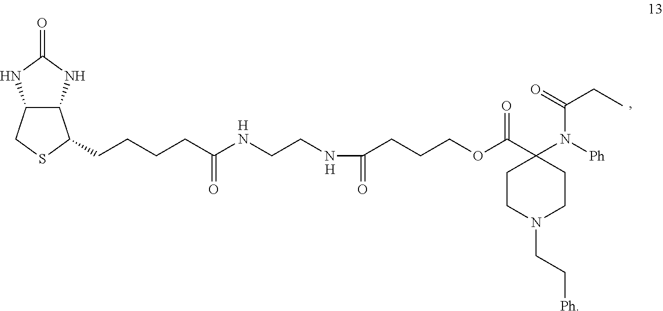

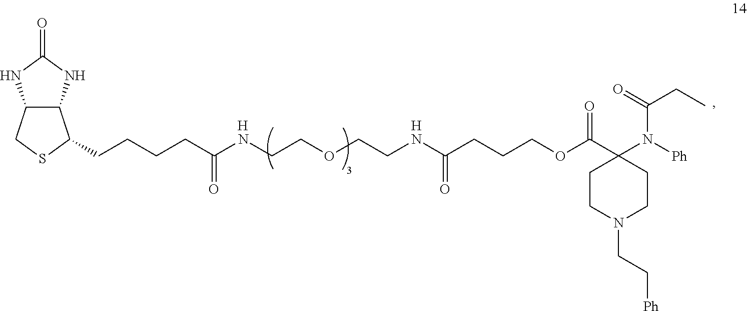

for ELISA analysis to isolate a series of high affinity, pan specific antibodies against fentanyl compounds.

### Embodiments 1 to 66

Embodiment 1. A compound of Formula (B1):

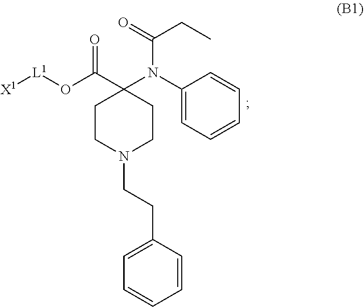

wherein: X¹ comprises a protein, an affinity moiety, a detectable moiety, a solid support, a leaving group, a protecting group, or a carrier; and L¹ is a bond, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

Embodiment 2. The compound of Embodiment 1, wherein L¹ is substituted or unsubstituted alkylene, or substituted or unsubstituted heteroalkylene.

Embodiment 3. The compound of Embodiment 2, wherein L¹ is substituted or unsubstituted C₂-C₂₄ alkylene, or substituted or unsubstituted 2 to 24 membered heteroalkylene.

Embodiment 4. The compound of Embodiment 3, wherein L¹ is substituted or unsubstituted C₂-C₁₈ alkylene, or substituted or unsubstituted 2 to 18 membered heteroalkylene.

Embodiment 5. The compound of Embodiment 4, wherein L¹ is substituted or unsubstituted C₂-C₁₂ alkylene, or substituted or unsubstituted 2 to 12 membered heteroalkylene.

Embodiment 6. The compound of Embodiment 5, wherein L¹ is substituted 2 to 12 membered heteroalkylene.

Embodiment 7. The compound of Embodiment 5, wherein the heteroalkylene comprises 1 or 2 nitrogen heteroatoms, and is substituted with 1 or 2 oxo.

Embodiment 8. The compound of Embodiment 1, wherein L¹ is —(CH₂)_{z}—C(O)—NH—(CH₂)_{z}NH— or —(CH₂)_{z}—C(O)—NH—(CH₂)_{z}—, wherein z is an integer from 1 to 6.

Embodiment 9. The compound of Embodiment 8, wherein L¹ is —(CH₂)₃—C(O)—NH—(CH₂)₂NH— or —(CH₂)₃—C(O)—NH—(CH₂)₂—.

Embodiment 10. The compound of Embodiment 1, wherein L¹ is —(CH₂)_{z}—C(O)— or —(CH₂)_{z}—CO—NH—, wherein z is an integer from 1 to 6.

Embodiment 11. The compound of Embodiment 10, wherein L¹ is —(CH₂)₃—C(O)— or —(CH₂)₃—CO—NH—

Embodiment 12. The compound of Embodiment 1, wherein L¹ is —(CH₂)_{z}—C(O)—NH—(CH₂)_{z}—(OCH₂CH₂)_{z}NH—, wherein each z independently an integer from 1 to 6.

Embodiment 13. The compound of Embodiment 12, wherein each z independently an integer from 1 to 4.

Embodiment 14. The compound of Embodiment 13, wherein each z independently an integer from 1 to 3.

Embodiment 15. The compound of Embodiment 12, wherein L¹ is —(CH₂)₃—C(O)—NH—(CH₂)₂—(OCH₂CH₂)₃NH—.

Embodiment 16. The compound of any one of Embodiments 1 to 15, wherein X¹ comprises a protein.

Embodiment 17. The compound of Embodiment 16, wherein the protein is albumin, tetanus toxoid, CRM197, keyhole limpet hemocyanin, diphtheria toxoid, Pseudomonas aeruginosa exoprotein A, cholera toxin subunit b, or flagellin.

Embodiment 18. The compound of any one of Embodiments 1 to 15, wherein X¹ comprises a detectable moiety; wherein the detectable moiety comprises a fluorophore or a magnetic bead.

Embodiment 19. The compound of any one of Embodiments 1 to 15 and 18, wherein X¹ comprises an affinity moiety.

Embodiment 20. The compound of Embodiment 19, wherein the affinity moiety comprises biotin, streptavidin, avidin, or a magnetic bead.

Embodiment 21. The compound of any one of Embodiments 1 or 16-20, wherein the compound of Formula (B1) is a compound of Formula (B2):

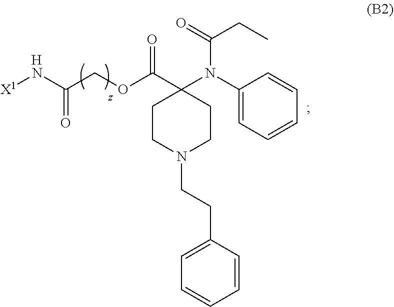

wherein X¹ comprises a protein, an affinity moiety, a detectable moiety, a solid support, a leaving group, a protecting group, or a carrier; and z is an integer from 1 to 6.

Embodiment 22. The compound of Embodiment 21, wherein z is an integer from 2 to 4.

Embodiment 23. The compound of Embodiment 22, wherein z is 3.

Embodiment 24. The compound of any one of Embodiments 1 or 16-20, wherein the compound of Formula (B2) is a compound of Formula (B3), (B4), or (B5):

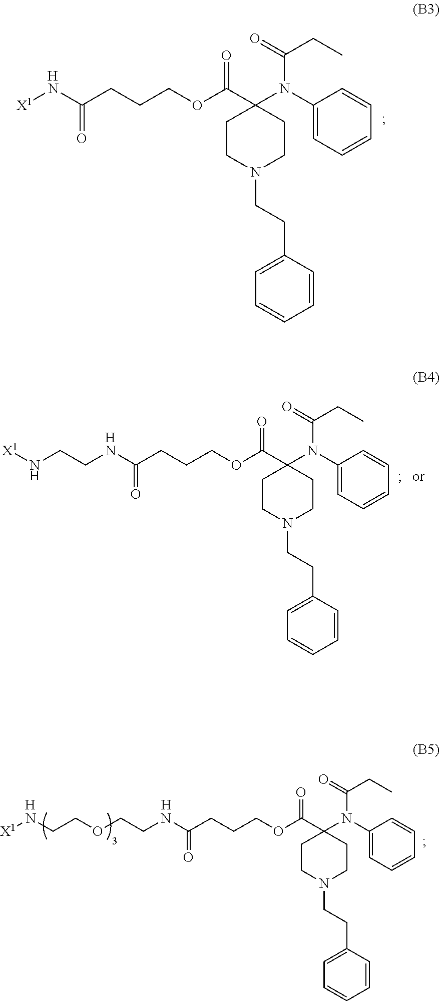

wherein X¹ comprises a protein, an affinity moiety, a detectable moiety, a solid support, a leaving group, a protecting group, or a carrier.

Embodiment 25. The compound of Embodiment 1, wherein the compound of Formula (B2) is a compound of Formula (B6) or (B7):

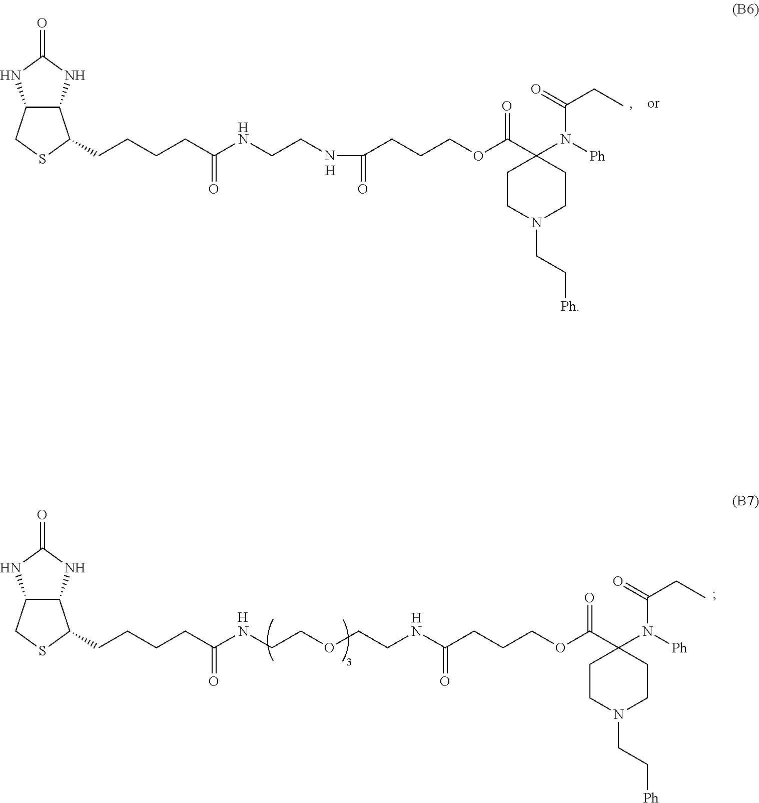

wherein Ph is phenyl.

Embodiment 26. A compound of Formula (A1):

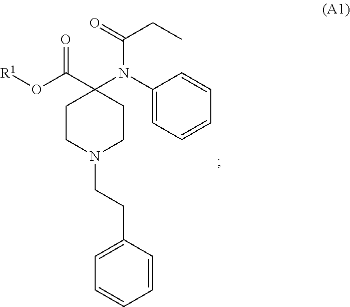

wherein R¹ is substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; with the proviso that R¹ is not methyl.

Embodiment 27. The compound of Embodiment 26, wherein R¹ is a substituted or unsubstituted to 2 to 16 membered heteroalkyl.

Embodiment 28. The compound of Embodiment 27, wherein R¹ is an unsubstituted to 2 to 12 membered heteroalkyl.

Embodiment 29. The compound of Embodiment 27, wherein R¹ is a substituted to 2 to 12 membered heteroalkyl, wherein the heteroalkyl is substituted with 1, 2, 3, or 4 moieties selected from the group consisting of oxo, —CCl₃, —CBr₃, —CF₃, —Cl₃, CHCl₂, —CHBr₂, —CHF₂, —CHI₂, —CH₂Cl, —CH₂Br, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, and —OCH₂F.

Embodiment 30. The compound of Embodiment 29, wherein the heteroalkyl is substituted with 1, 2, or 3 moieties selected from the group consisting of oxo, —CN, —OH, —NH₂, —COOH, —CONH₂, —NO₂, —NHNH₂, —ONH₂, —NHC(O)NHNH₂, —NHC(O)NH₂, —NHC(O)H, —NHC(O)OH, and —NHOH.

Embodiment 31. The compound of Embodiment 30, wherein the heteroalkyl is substituted with 1, 2, or 3 moieties selected from the group consisting of oxo, —OH, —NH₂, —COOH, and —CONH₂.

Embodiment 32. The compound of Embodiment 26, wherein R¹ is a substituted alkyl, wherein the alkyl is substituted with 1, 2, 3, or 4 moieties selected from the group consisting of oxo, —CCl₃, —CBr₃, —CF₃, —Cl₃, CHCl₂, —CHBr₂, —CHF₂, —CHI₂, —CH₂Cl, —CH₂Br, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, and —OCH₂F.

Embodiment 33. The compound of Embodiment 32, wherein the alkyl is substituted with 1, 2, 3, or 4 moieties selected from the group consisting of oxo, —CN, —OH, —NH₂, —COOH, —CONH₂, —NO₂, —NHNH₂, —ONH₂, —NHC(O)NHNH₂, —NHC(O)NH₂, —NHC(O)H, —NHC(O)OH, and —NHOH.

Embodiment 34. The compound of Embodiment 33, wherein the alkyl is substituted with 1, 2, or 3 moieties selected from the group consisting of oxo, —OH, —NH₂, —COOH, and —CONH₂.

Embodiment 35. The compound of any one of Embodiments 32 to 34, wherein R¹ is substituted C₂-C₁₂ alkyl.

Embodiment 36. The compound of Embodiment 26, wherein R¹ is —(CH₂)_{z}—COOH, wherein z is an integer from 1 to 6.

Embodiment 37. The compound of Embodiment 36, wherein the compound of Formula (A1) is a compound of Formula (A2):

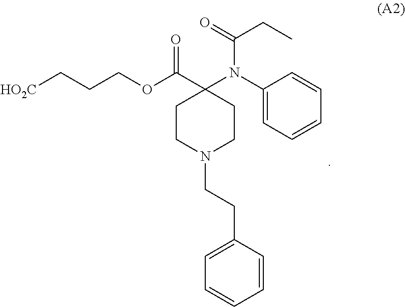 [SMILES: CCC(=O)N(c1ccccc1)C1(C(=O)OCCCC(=O)O)CCN(CCc2ccccc2)CC1]

Embodiment 38. A vaccine comprising the compound of any one of Embodiments 1-18, 21-24, and 26-37, and a pharmaceutically acceptable adjuvant, a pharmaceutically acceptable excipient, or a combination thereof.

Embodiment 39. The vaccine of Embodiment 38, wherein the pharmaceutically acceptable adjuvant comprises an aluminum salt.

Embodiment 40. The vaccine of Embodiment 39, wherein the aluminum salt is aluminum sulfate, aluminum phosphate, aluminum hydroxyphosphate, aluminum hydroxide, potassium aluminum sulfate, or a combination of two or more thereof.

Embodiment 41. The vaccine of any one of Embodiments 38 to 40, wherein the pharmaceutically acceptable adjuvant comprises a toll-like receptor agonist.

Embodiment 42. The vaccine of Embodiment 41, wherein the toll-like receptor is toll-like receptor 2 agonist, toll-like receptor 3 agonist, toll-like receptor 4 agonist, toll-like receptor 5 agonist, toll-like receptor 7 agonist, toll-like receptor 8 agonist, toll-like receptor 9 agonist, or a combination of two or more thereof.

Embodiment 43. The vaccine of Embodiment 42, wherein the toll-like receptor agonist is toll-like receptor 9 agonist.

Embodiment 44. The vaccine of Embodiment 43, wherein toll-like receptor 9 agonist is a CpG ODN.

Embodiment 45. The vaccine of Embodiment 44, wherein the CpG ODN is a CpG-A ODN, a CpG-B ODN, a CpG-C ODN, or a combination of two or more thereof.

Embodiment 46. The vaccine of Embodiment 44, wherein the CpG ODN is CpG ODN 1585, CpG ODN 2216, CpG ODN 2336, CpG ODN 1668, CpG ODN 1826, CpG ODN 2006, CpG ODN 2007, CpG ODN BW006, CpG ODN D-SL01, CpG ODN 2395, CpG ODN M362, CpG ODN D-SL03, or a combination of two or more thereof.

Embodiment 47. A method of treating opioid use disorder and/or preventing opioid use disorder and/or preventing an opioid overdose in a subject in need thereof, the method comprising administering to the subject an effective amount of the compound of any one of Embodiments 1-18, 21-24, and 26-37 or the vaccine of any one of Embodiments 38 to 46; wherein the opioid is carfentanil or a carfentanil analogue.

Embodiment 48. The method of Embodiment 47 for treating opioid use disorder.

Embodiment 49. The method of Embodiment 47 for preventing opioid use disorder.

Embodiment 50. The method of any one of Embodiments 47 to 49 for preventing opioid overdose.

Embodiment 51. A method for generating IgG antibodies to carfentanil or a carfentanil analogue in a subject in need thereof, the method comprising administering to a subject an effective amount of the compound of any one of Embodiments 1-18, 21-24, and 26-37 or the vaccine of any one of Embodiments 38 to 46.

Embodiment 52. A method of isolating an antibody to carfentanil or a carfentanil analogue, the method comprising: (i) administering to a subject an effective amount of: (a) the compound of any one of Embodiments 1-18, 21-24, and 26-37, wherein X¹ is a protein; or (b) the vaccine of any one of Embodiments 38 to 46, wherein X¹ is a protein, to produce an antibody to carfentanil or a carfentanil analogue; and (ii) isolating the antibody.

Embodiment 53. The method of Embodiment 52, wherein (ii) comprises screening the antibody using the compound of any one of Embodiments 1-15, and 18-25, wherein X¹ comprises an affinity moiety or a detectable moiety, to isolate the antibody.

Embodiment 54. A method of isolating an antibody to carfentanil or a carfentanil analogue, the method comprising: (i) administering to a subject an effective amount of: (a) a compound comprising a moiety of Formula (C1); (b) a compound comprising a protein and a moiety of Formula (C1); (c) a vaccine comprising a compound which comprises a moiety of Formula (C1); or (d) a vaccine comprising a compound which comprises a protein and a moiety of Formula (C1), to produce an antibody to carfentanil or a carfentanil analogue; and (ii) isolating the antibody; wherein the moiety of Formula (C1) is:

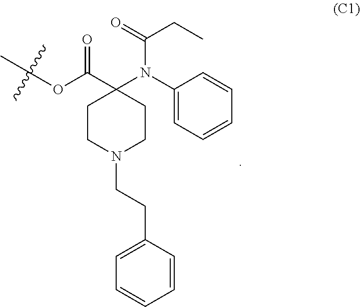 [SMILES: *OC(=O)C1(N(C(=O)CC)c2ccccc2)CCN(CCc2ccccc2)CC1]

Embodiment 55. The method of Embodiment 54, wherein (ii) comprises screening the antibody using a compound which comprises an affinity moiety and/or a detectable moiety and a moiety of Formula (C1), wherein the moiety of Formula (C1) is:

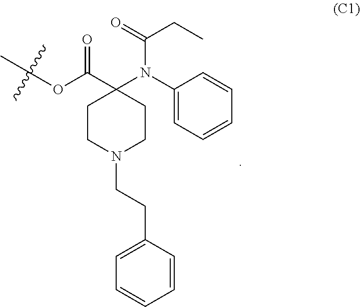 [SMILES: *OC(=O)C1(N(C(=O)CC)c2ccccc2)CCN(CCc2ccccc2)CC1]

Embodiment 56. The method of any one of Embodiments 47 to 55, wherein the carfentanil analogue is sufentanil, remifentanil, alfentanil, lofentanil, brifentanil, or trefentanil.

Embodiment 57. A method of isolating an antibody to fentanyl or a fentanyl analogue, the method comprising: (i) administering to a subject an effective amount of: (a) a compound comprising a moiety of Formula (E); (b) a compound comprising a protein and a moiety of Formula (E); (c) a vaccine comprising a compound which comprises a moiety of Formula (E); or (d) a vaccine comprising a compound which comprises a protein and a moiety of Formula (E), to produce an antibody to fentanyl or a fentanyl analogue; and (ii) isolating the antibody; wherein the moiety of Formula (E) is:

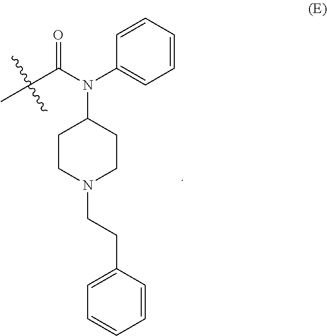 [SMILES: *C(=O)N(c1ccccc1)C1CCN(CCc2ccccc2)CC1]

Embodiment 58. The method of Embodiment 57, wherein (ii) comprises screening the antibody using a compound which comprises an affinity moiety and/or a detectable moiety and a moiety of Formula (E), wherein the moiety of Formula (E) is:

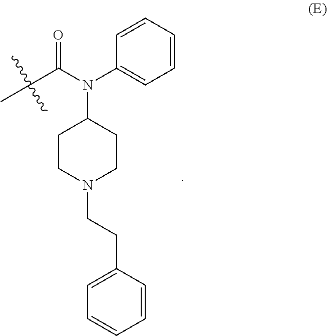 [SMILES: *C(=O)N(c1ccccc1)C1CCN(CCc2ccccc2)CC1]

Embodiment 59. The method of Embodiment 57 or 58, wherein the compound comprising the moiety of Formula (E) is a compound of Formula (D1):

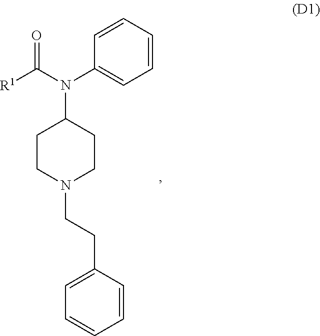

wherein R¹ substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

Embodiment 60. The method of Embodiment 58 or 59, wherein the compound comprising the detectable moiety and/or the affinity moiety and the moiety of Formula (E) is a compound of Formula (D2):

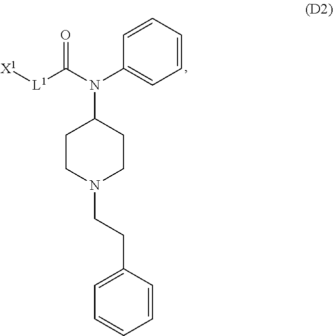

wherein X¹ is a detectable moiety and/or an affinity moiety; and L¹ is a bond, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

Embodiment 61. The method of Embodiment 59 or 60, wherein the compound of Formula (D1) is a compound of Formula (D3):

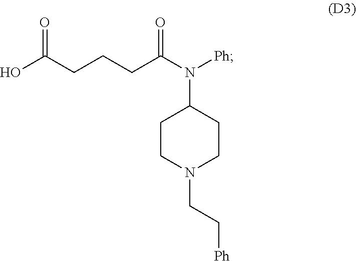

wherein Ph is phenyl.

Embodiment 62. The method of Embodiment 60 or 61, wherein the compound of Formula (D2) is a compound of Formula (D4) or (D5):

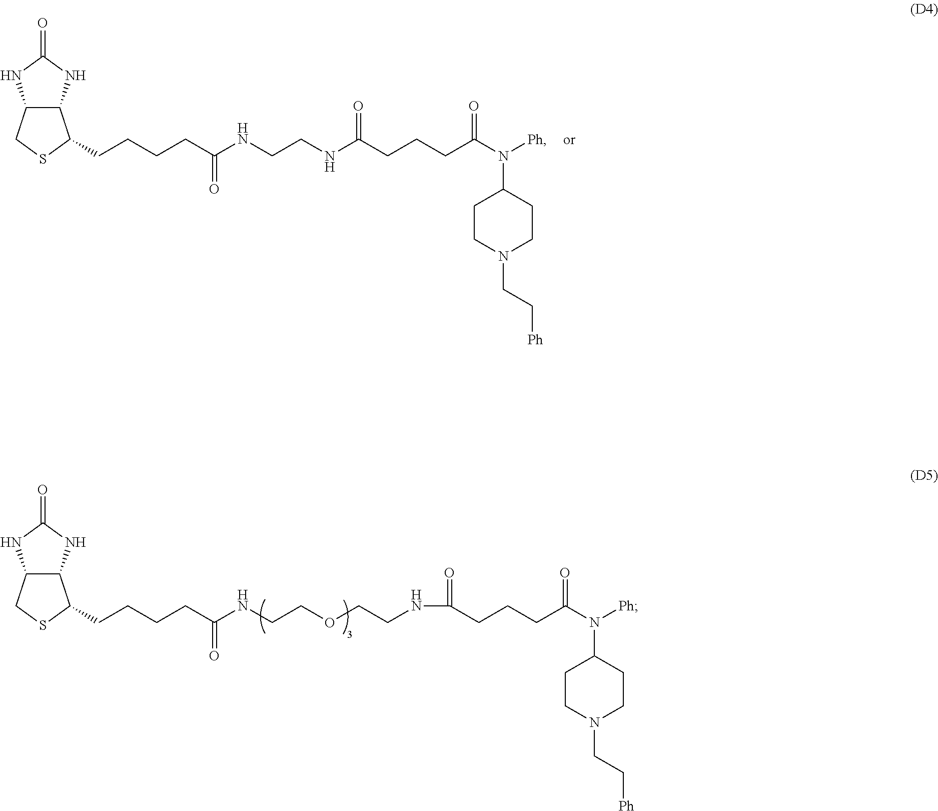

wherein Ph is phenyl.

Embodiment 63. The method of any one of Embodiments 57 to 62, wherein the fentanyl analogue is acetylfentanyl, butyrfentanyl, para-tolylfentanyl, 3-methylfentanyl, α-methylfentanyl, mefentanyl, phenaridine, ohmefentanyl, or mirfentanil.

Embodiment 64. A compound of Formula (D4) or (D5):

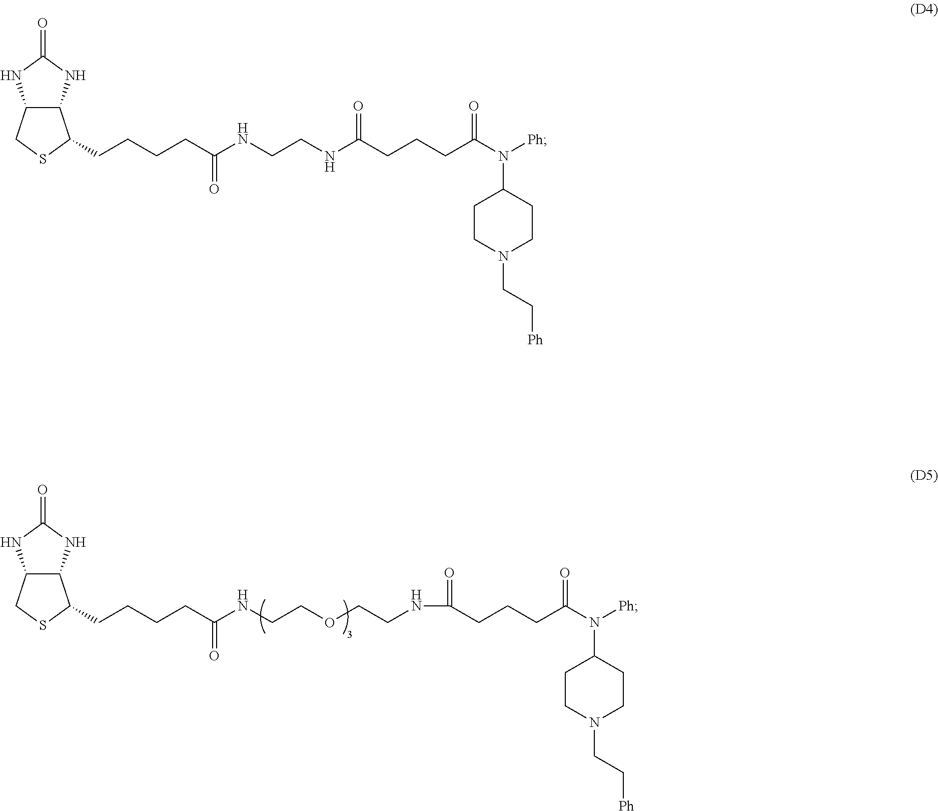

wherein Ph is phenyl.

Embodiment 66. A compound of the formula:

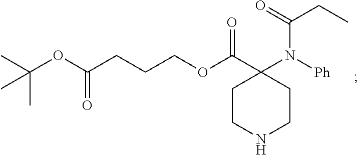

wherein Ph is phenyl.

Embodiment 67. A compound of Formula (B1):

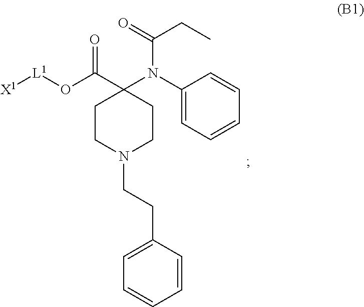

wherein: L¹ is a 2 to 12 membered heteroalkylene comprising 1 or 2 nitrogen heteroatoms, and substituted with 1 or 2 oxo; and X¹ is keyhole limpet hemocyanin, serum albumin, tetanus toxoid, CRM197, biotin, a fluorophore, a streptavidin-fluorophore, or an avidin-fluorophore.

Embodiment 68. A vaccine comprising the compound of Embodiment 67, an aluminum salt, a toll-like receptor agonist, and a pharmaceutically acceptable excipient.

Embodiment 69. A method for generating IgG antibodies to carfentanil or a carfentanil analogue in a subject in need thereof, the method comprising administering to the subject an effective amount of the vaccine of Embodiment 68.

### EXAMPLES

The following examples are for the purposes of illustration only, and are not intended to limit the spirit or scope of the disclosure or claims.

### Example 1

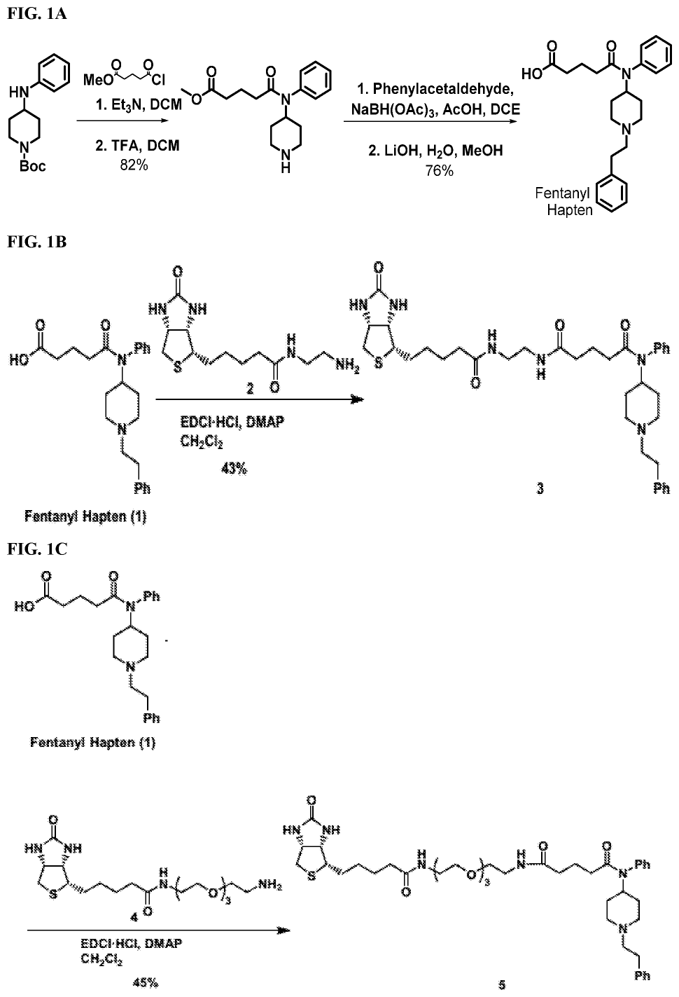

FIGS. 1A-1B show the chemical synthesis of two probes that are representative of the fentanyl-like scaffolding. Both probes were prepared from the fentanyl hapten 1 that was used for vaccination. Probe 3 displays a short linker with a biotin tag for selection (FIG. 1). Probe 5 also presents the fentanyl scaffolding where biotin is appended with a PEG linker of three units (FIG. 1C).

### Example 2

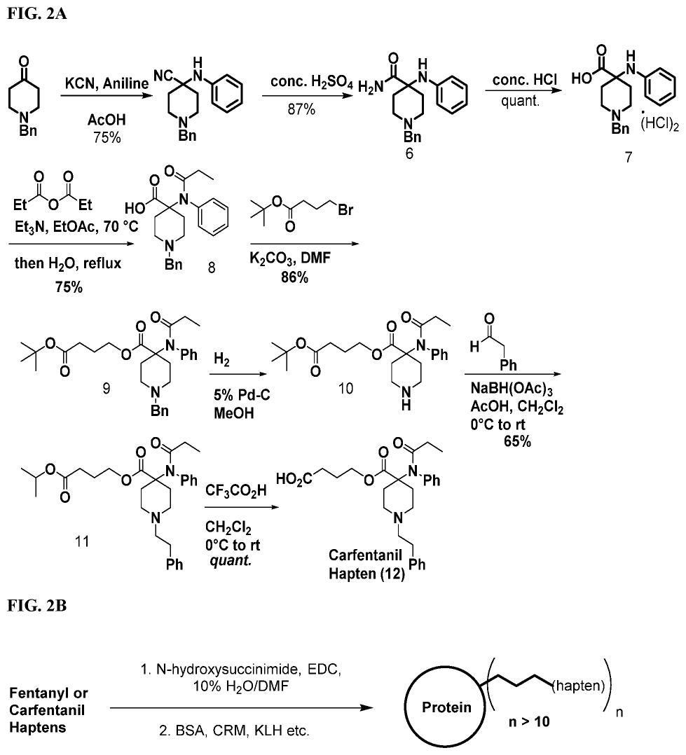

FIG. 2A shows the chemical synthesis of the compounds described herein, which provides a carfentanil-like scaffolding. The reasoning behind using a second class of synthetic opioid probes is that carfentanil is almost 10,000 times more potent than morphine. The potential danger here from overdose with carfentanil contaminated heroin samples as well as carfentanil's well known use as a chemical warfare agent makes antibody selection to this drug tantamount. The argument that naloxone can be used to reverse opioid toxicity is not valid with carfentanil because naloxone therapy has insufficient efficacy in reversing a carfentanil overdose due to the extended half-life of carfentanil. With a selected antibody fragment, there is an advantage of being able to tune the antibodies half-life to exceed that of carfentanil. This will provide a window for protection when naloxone's therapeutic benefit wanes.

FIG. 2B shows the chemical synthesis of a fentanyl hapten (1) or carfentanil hapten (12) covalently linked to a protein (e.g., tetanus toxoid, bovine serum albumin, CRM197, KLH). The skilled artisan will appreciate that a protein can be covalently linked to a plurality of haptens. These haptens will be used for vaccination (e.g., a carfentanil-tetanus toxoid conjugate) and ELISA assays (e.g., a carfentanil-bovine serum albumin conjugate).

### Example 3

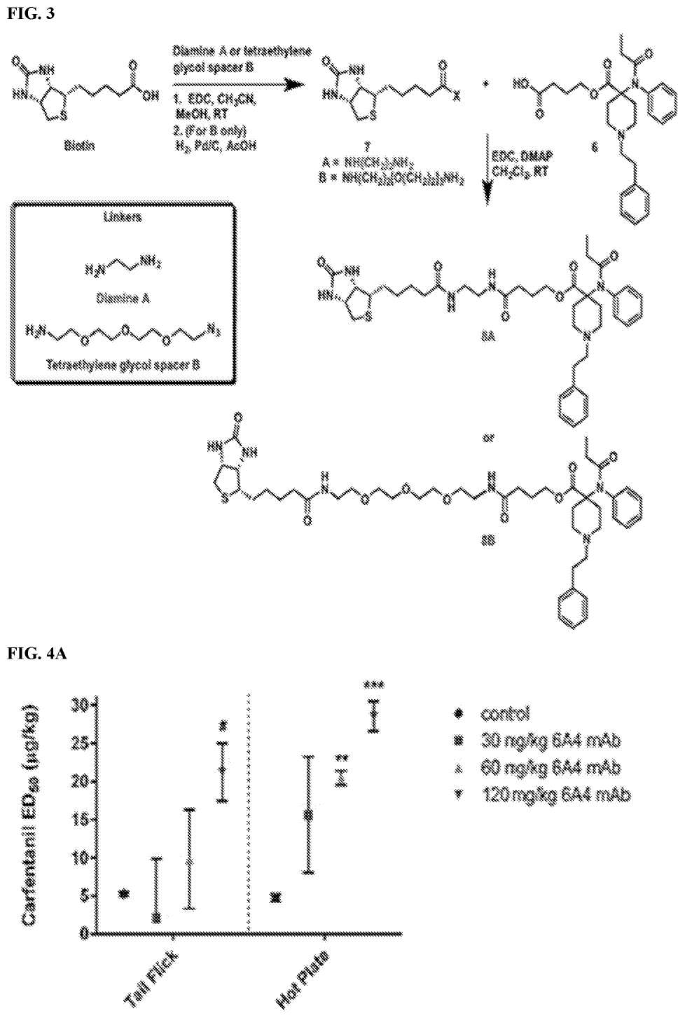

FIG. 3 shows the chemical synthesis and structures of a probes of the compounds as described herein that are representative of the carfentanil-like scaffolding, where the probe can be used for antibody selection. To overcome the tendency for dimerization in the first step with diamine A, a second method was employed using a tetraethylene glycol spacer (B). After selective coupling, the azide in the B linker was then reduced to give free amine. Both linkers have been prepared in 5 mg scale quantities, with overall yields of 13 and 32%, respectively for 8A and 8B, starting from hapten 6. Subsequently, Linkers 8A and 8B will be conjugated to fluorescent streptavidin probes, which are commercially available and can range from 350 nm to 750 nm. Streptavidin, Texas Red conjugate (λₑₓ=596 nm/λₑₘ=612 nm) will be used for screening. The fluorescently labeled carfentanil haptens will then be used for hapten-specific B-cell sorting by fluorescence-activated cell sorting (FACS).

### Example 4

One discovery herein described is a process that allows for the isolation of antibodies with both high affinity, yet, pan specificity to all of the fentanyl class of drugs. The key aspects of developing a monoclonal antibody against the fentanyl drug class is that it would provide immediate changes in both in the amount and the rate of fentanyl entry into medically critical sites of action in the CNS and cardiovascular system. Furthermore, depending on the antibody fragment used the effects could be long lasting.

One discovery herein described is the design and preparation of chemical probes that can be used for the selection of high affinity; yet pan specificity antibodies against the fentanyl class of synthetic opioids. While this would seem to be a trivial exercise, it is not as the goal here will be to identify a therapeutic monoclonal antibody against not only fentanyl but one that has cross reactivity to all the other derivatives in the 4-anilidopiperidine drug class with high affinity. The key aspects of designing probes for antibody-fentanyl selection is that they must preserve the critical chemical epitopes that are produced upon immunization with a haptenic structure. Moreover, such probes must possess regiochemical functionality so as to allow attachment of selectable functionality for antibody screening and ultimately antibody selection without impeding on the selection process. The following two synthetic schemes show the probes used for fentanyl class antibody screening as well as details of their synthesis.

Vaccination with fentanyl hapten 1 and carfentanil hapten 12 in both mice and rats has allowed antibody induction to a fentanyl class of antibody binders. The versatility of these probes is that they can be used with streptavidin coated plates (ELISA selection), Surface Plasma Resonance (SPR) chips coated with streptavidin (SPR selection) or streptavidin fluorescent labeled probes for fluorescence-activated cell sorting (FACS) antibody selection. Using fentanyl Hapten 1 for mouse immunization and probes 13 and 14 for ELISA analysis allows for the isolation of a series of high affinity, yet, pan specific antibodies. As shown in Table 1, the antibodies have excellent affinity to fentanyl derivatives as determined by SPR.

TABLE 1

| Hybridoma | | | | | | | |
|---|---|---|---|---|---|---|---|
| Code | Carfentanil | Fentanyl | 3-Me | α-Me | Ac | Bu | Tol |
| 1 | 5 | 1 | 5 | 5 | 5 | 5 | 5 |
| 2 | 5 | 1 | 5 | 1 | <1 | 1 | 5 |
| 3 | <1 | <1 | 50 | 5 | 1 | <1 | 100 |
| 4 | 1 | 5 | 5 | 5 | 1 | <1 | 50 |
| 5 | >>100 | 5 | 5 | 5 | 5 | 5 | 5 |

With reference to Table 1, “3-Me” is 3-methylfentanyl; “α-Me” is α-methylfentanyl; “Ac” is acetylfentanyl; “Bu” is butyrfentanyl; and “Tol” is para-tolylfentanyl.

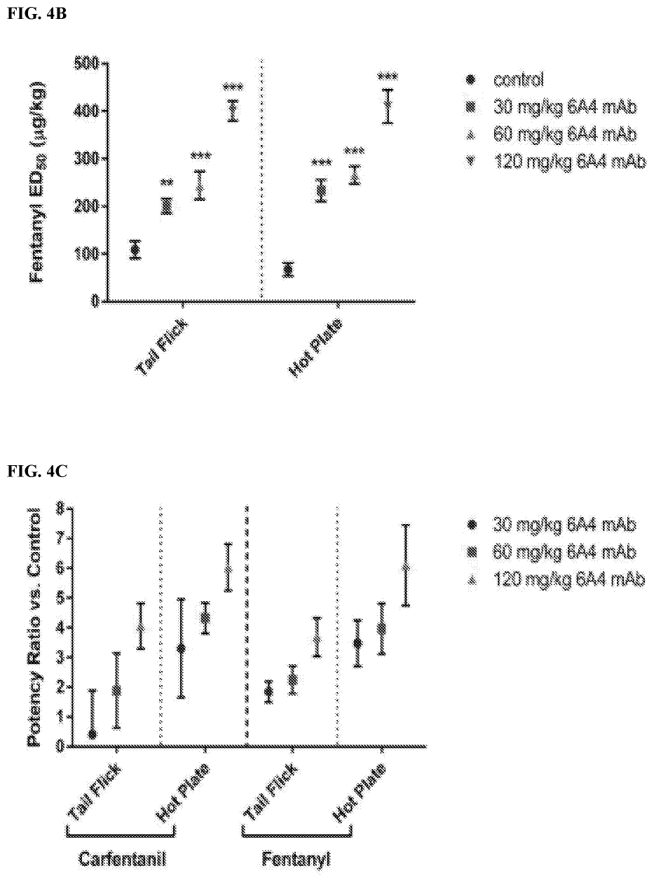

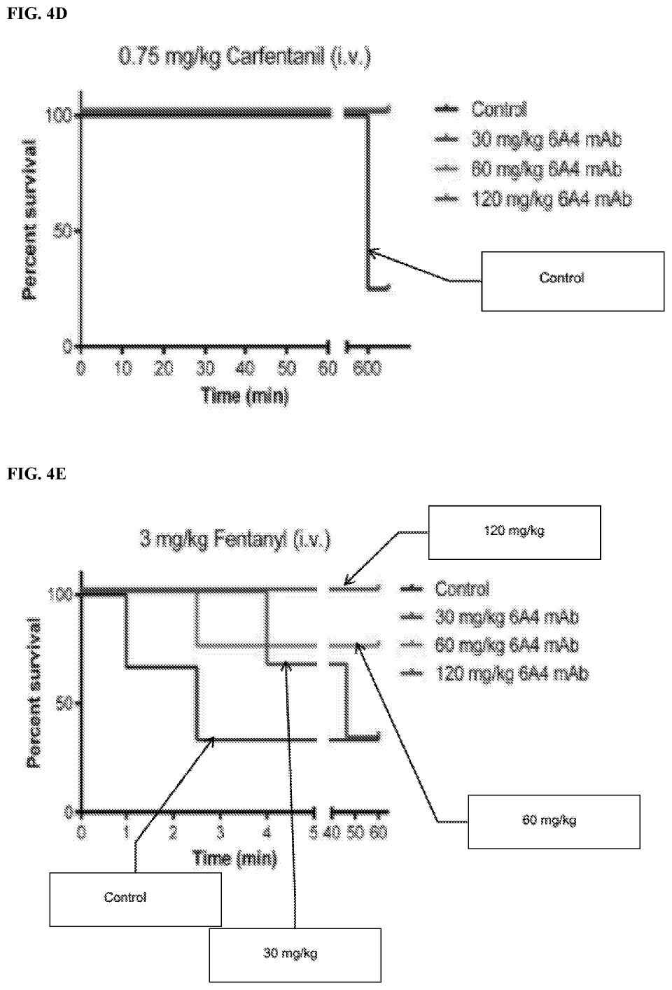

A second testimony to the value of this selection process comes with the examination of one of the antibodies. Here hot plate and tail flick antinociception was used as a surrogate for drug reward because it is mediated in the central nervous system and provides a relevant behavioral model of the antibodies ability to reduce drug access to brain and its subsequent effects (FIGS. 4A-4B). ED₅₀ values reflect the effective dose of drug where half of the animals in a group experience the full antinociceptive effect of the opioid. Potency ratios were calculated by dividing the vaccine-shifted ED₅₀ value from the control values in each antinociceptive test (FIG. 4C). From the data shown in FIGS. 4A-4E, both carfentanil and fentanyl were examined with this antibody. A critical piece of data demonstrating the validity of these probes for fentanyl-antibody selection is the percent survival seen with both drugs (FIGS. 4D-4E). Based on the dose of antibody used, it was remarkable and unexpected that complete protection from a lethal overdose of carfentanil (FIG. 4D) and fentanyl (FIG. 4E) was seen.

### Example 5

A vaccine was prepared with 50 μg of a CRM197-carfentanil hapten conjugate; 30 μg CpG ODN 1826+100 μg Alum (150 μl of CRM197-carfentanil hapten conjugate/CpG ODN 1826/Alum per mouse); where the CRM197-carfentanil hapten conjugate copy number=15. The control vaccine contained 50 μg keyhole limpet hemocyanin (KLH)+30 μg CpG ODN 1826+100 μg Alum (150 μl of KLH/CpG/Alum per mouse). The CRM197-carfentanil hapten conjugate is represented by the following structure (where Ph is phenyl) and can be prepared by the methods shown in FIGS. 2A-2B:

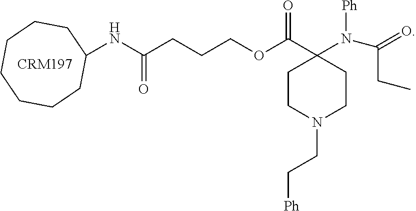 [SMILES: CCC(=O)N(c1ccccc1)C1(C(=O)OCCCC(=O)NC2CCCCCCC2)CCN(CCc2ccccc2)CC1]

The vaccine study was conducted with 6-8 week old male Swiss Webster mice who were subcutaneously administered the CRM197-carfentanil hapten conjugate vaccine or the control vaccine at weeks 0, 2, and 4. Sample bleeds occurred at weeks 3 and 5; an antinociception assay was conducted at week 6; and biodistribution studies were conducted at week 7.

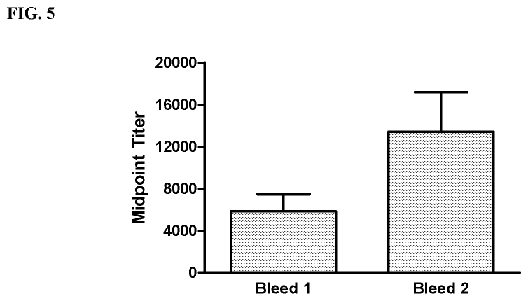

The results of the antibody titers at week 3 (Bleed 1) and week 5 (Bleed 2) are shown in Table 2 below and in FIG. 5.

TABLE 2

| | Mean Antibody Titer |
|---|---|
| Bleed 1 (Week 3) | 5847 |
| Bleed 2 (Week 5) | 13428 |

Surface Plasmon Resonance was used to test anti-opioid antibody binding affinity. The results are shown in Table 3.

TABLE 3

| | IC₅₀ Value |
|---|---|
| Carfentanil | 15.2 nM |
| Fentanyl | 277 |

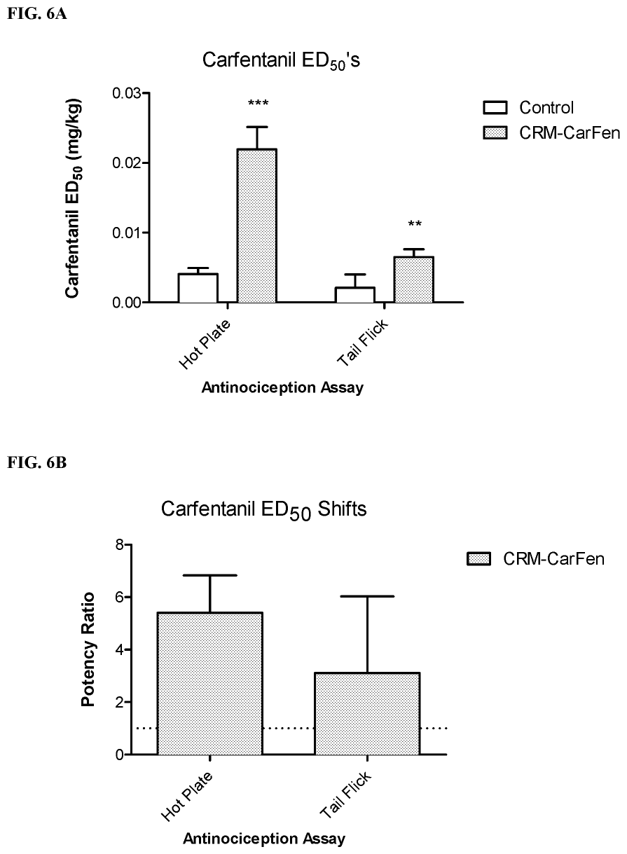

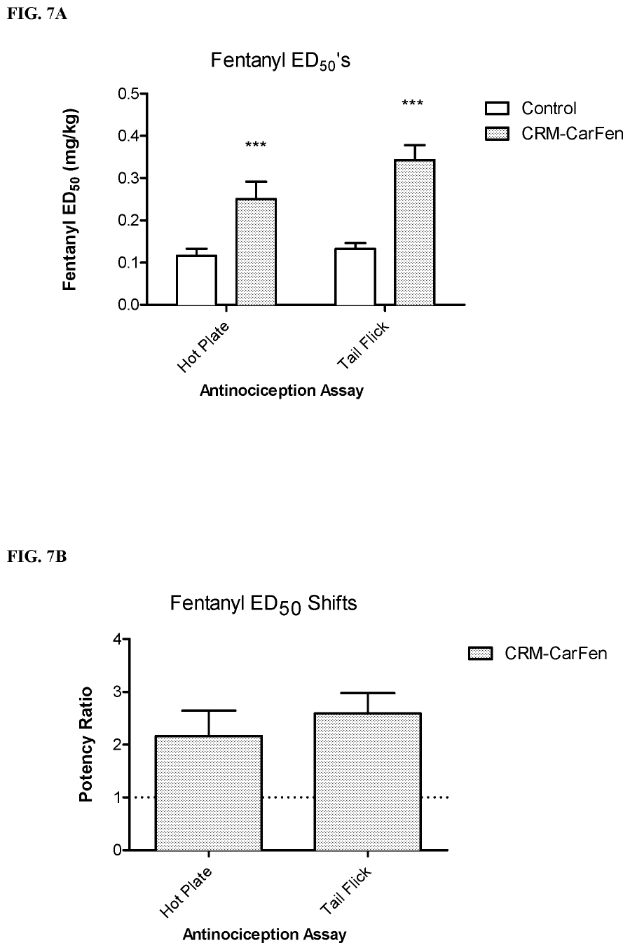

Hot plate and tail flick antinociception was used as a surrogate for drug reward because it is mediated in the central nervous system and provides a relevant behavioral model of the ability of the CRM197-carfentanil hapten conjugate vaccine to reduce opioid (carfentanil or fentanyl) access to the brain (e.g., to the mu-opioid receptors in the brain) and its subsequent effects (FIGS. 6A-6B; FIGS. 7A-7B). ED₅₀ values reflect the effective dose of drug where half of the animals in a group experience the full antinociceptive effect of the opioid (FIG. 6A; FIG. 7A). Potency ratios were calculated by dividing the vaccine-shifted ED₅₀ value from the control values in each antinociceptive test (FIG. 6B; FIG. 7B).

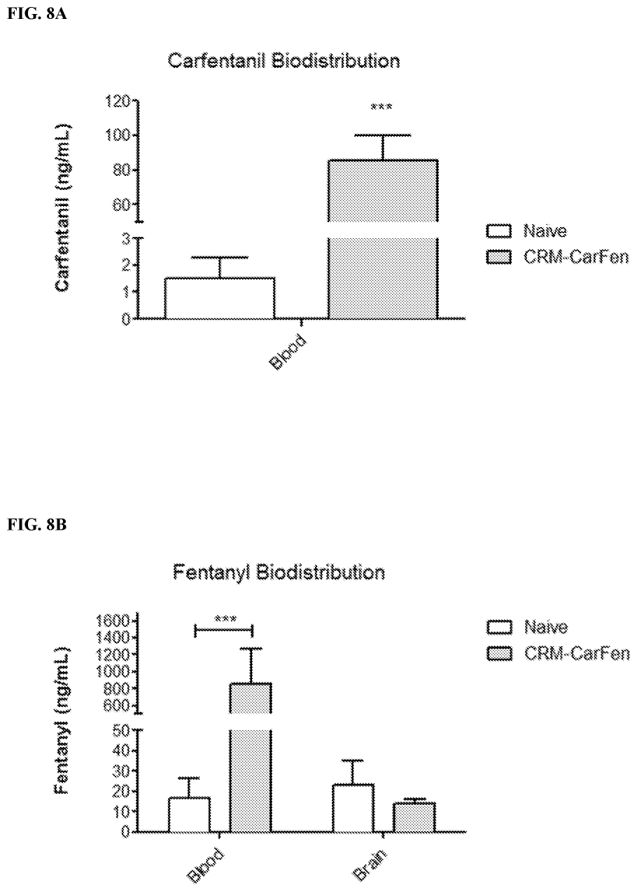

The results of the biodistribution studies in the blood with either carfentanil (0.02 mg/kg) or the blood and brain with fentanyl (0.2 mg/kg) are shown in FIGS. 8A-8B. As can be seen from the results, the CRM197-carfentanil hapten conjugate vaccine significantly increased the blood concentration of carfentanil. The more carfentanil that is bound in the blood by antibodies generated by the vaccines described herein, the less carfentanil can enter the brain and bind to the mu-opioid receptor, thus preventing and/or reducing the incidence of opioid overdose and/or opioid addiction in a subject

### Example 7

Carfentanil-KLH conjugates were prepared (e.g., FIGS. 2A-2B) by reacting the carfentanil hapten with keyhole limpet hemocyanin (KLH) to produce a compound of the structure:

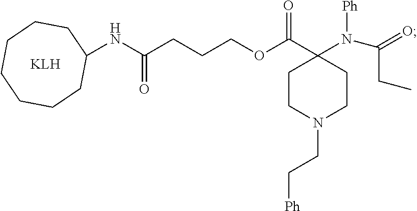

where Ph is phenyl and “KLH” represents keyhole limpet hemocyanin. A vaccine was prepared by combining the carfentanil-KLH conjugate (100 μg) with alum (1 mg) and CpG ODN 1826 (100 μg).

Two rats were immunized with the carfentanil-KLH conjugate (100 μg) adjuvanted with alum (1 mg) and CpG ODN 1826 (100 μg) at months 0, 1, 2, and 3, and serum samples (bleeds) were taken at months 1.5, 2.5 and 3.5. Serum antibodies were monitored as shown below to confirm the presence of high affinity anti-opioid antibodies.

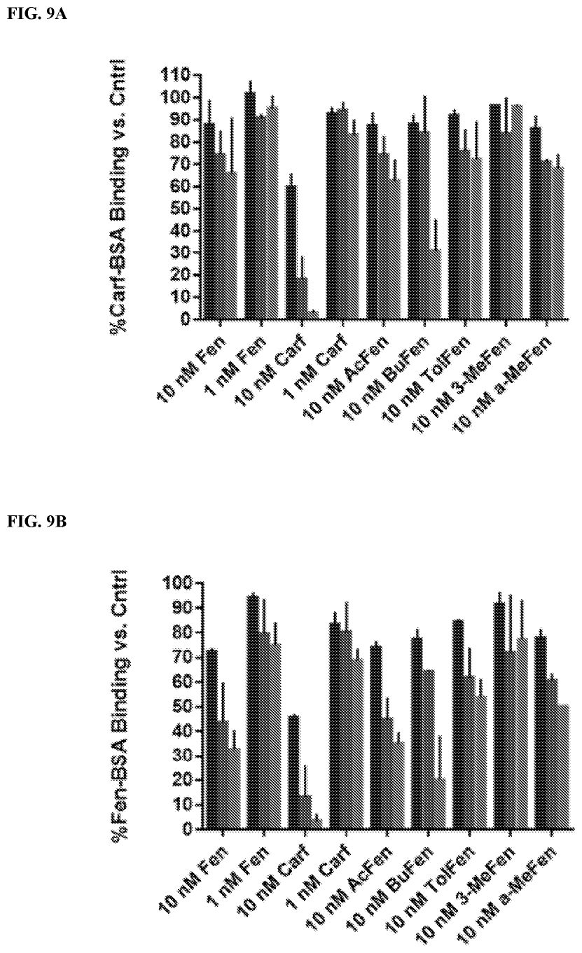

Serum samples from the three bleeds from the two immunized rats were diluted to 1:3000 (rat #2) or 1:8000 (rat #1) into running buffer, then subjected to assay conditions. Binding signals expressed in resonance units (RU) were normalized to RU values obtained without competitor molecules. Antibody affinities increased throughout the immunization period yielding serum antibodies with <10 nM affinity for carfentanil and fentanyl prior to tissue harvest. These results are shown in FIGS. 9A-9B.

At month 4, an IV injection of the carfentanil-KLH conjugate was administered, and three days later the spleen and lymph nodes were harvested from the two rats and homogenized. Lymphocytes were isolated from the homogenate and sorted using the following strategy shown in Table 4.

TABLE 4

| | Population of | |
|---|---|---|
| Scatterplot | Interest | Gate |
| FSC × SSC | Lymphocytes | P1 |
| FSC (H) × FSC (W) | Singlets | P2 fom P1 |
| SSC (W) × | FVS510− | P3 from P2 |
| FVS510(Live/dead) | | |
| PE-Cy7 (B cells) × | PE-Cy7+/ | P4 from P3 |
| PerCP (T cells) | PerCP− | |
| SSC (W) × FITC (IgG1-2a-2b) | FITC+ | P5 from P4 |
| BV421 (Carfentanil-(PEG)3) × | Double++ | P6 from P5 |
| AF647 (Carfentanil) | | |
| BUV395 (Fentanyl-(PEG)3 × | Double++ | P7 from P5 |
| PE (Fentanyl) | | |
| AF647 (Carfentanil) × | Double++ | P8 from [P6 or |
| PE (Fentanyl) | | P7] gate |

*Monitor P8 and index sort [P6 or P7] gate

Fentanyl-biotin probes and carfentanil-biotin probes (FIG. 1, FIG. 3) were prepared in separate tubes (with a 2:1 ratio of probe to streptavidin-fluorophore) and were combined in the staining mix at 10 nM final compound concentration. Single cells were collected in 1 plate for one rat and 1.5 plates for the second rat. Reverse transcriptase PCR was applied to each B-cell to generate cDNA, which was then used for next generation sequencing (NGS) and nested PCR. NGS results revealed 146 positive B-cells isolated from the sort. PCR products generated using primers designed to amplify the variable region of heavy chain, kappa chain or lambda chain were ligated via Gibson Assembly® method into expression vectors already containing the corresponding constant region sequences. The vectors also contained a carbenicillin resistance gene, and following transformation into E. coli, gave rise to viable colonies in the presence of antibiotic. Cloned plasmids isolated from the colonies via miniprep were then sequenced to verify HC or LC gene identity and that the genes were cloned in frame. Next, 146 HC/LC plasmid pairs were produced in small scale and transfected into HEK293 cells. Supernatants from cell culture were screened directly for immobilized carfentanil-BSA and fentanyl-BSA binding by SPR. Of the 146 total supernatants, 111 tested positive for binding by this method and were subsequently screened by an SPR competitive binding assay between free fentanyl compounds and the carfentanil/fentanyl coating antigens. Lastly, monoclonal antibodies were produced and purified for further affinity profiling: they showed subnanomolar K_{D} for both fentanyl and carfentanil and nanomolar affinity for related analogues such as butyrfentanyl, acetylfentanyl, 3-methylfentanyl, and α-methylfentanyl.

While various embodiments and aspects are shown and described herein, it will be clear to the skilled artisan that such embodiments and aspects are provided by way of example. Variations, changes, and substitutions will occur to the skilled artisan. It will be understood that various alternatives to the embodiments described herein can be used. All publications and patents cited herein are incorporated by reference herein in their entirety.

1-68. (canceled)

69. A compound of Formula (B2): 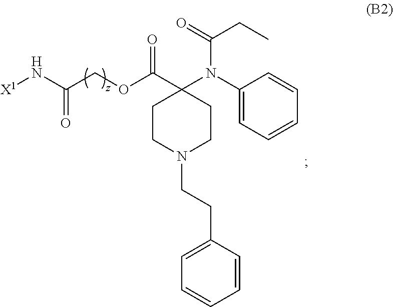
wherein:
X¹ is keyhole limpet hemocyanin, albumin, tetanus toxoid, CRM197, diphtheria toxoid, Pseudomonas aeruginosa exoprotein A, cholera toxin subunit b, flagellin, or a synthetic peptide carrier; and
z is an integer from 1 to 6.

70. The compound of claim 69, wherein X¹ is keyhole limpet hemocyanin.

71. The compound of claim 69, wherein X¹ is tetanus toxoid.

72. The compound of claim 69, wherein X¹ is CRM197.

73. The compound of claim 69, wherein X¹ is diphtheria toxoid.

74. The compound of claim 69, wherein X¹ is a synthetic peptide carrier.

75. The compound of claim 69, wherein z is an integer from 2 to 4.

76. The compound of claim 69, wherein z is an integer of 3.

77. A vaccine comprising the compound of claim 69 and an aluminum salt.

78. The vaccine of claim 77, wherein the aluminum salt is aluminum sulfate, aluminum phosphate, aluminum hydroxyphosphate, aluminum hydroxide, or potassium aluminum sulfate.

79. The vaccine of claim 77, wherein the aluminum salt is aluminum phosphate or aluminum hydroxide.

80. The vaccine of claim 77, further comprising a toll-like receptor agonist.

81. The vaccine of claim 80, wherein the toll-like receptor agonist is a CpG oligodeoxynucleotide (ODN).

82. The vaccine of claim 81, wherein the CpG ODN is CpG ODN 1585, CpG ODN 2216, CpG ODN 2336, CpG ODN 1668, CpG ODN 1826, CpG ODN 2006, CpG ODN 2007, CpG ODN BW006, CpG ODN D-SL01, CpG ODN 2395, CpG ODN M362, or CpG ODN D-SL03.

83. The vaccine of claim 81, wherein the CpG ODN is CpG ODN 2006.

84. A vaccine comprising the compound of claim 76 and an aluminum salt.

85. The vaccine of claim 84, wherein the aluminum salt is aluminum sulfate, aluminum phosphate, aluminum hydroxyphosphate, aluminum hydroxide, or potassium aluminum sulfate.

86. The vaccine of claim 84, further comprising a toll-like receptor agonist.

87. The vaccine of claim 86, wherein the toll-like receptor agonist is a CpG oligodeoxynucleotide (ODN).

88. The vaccine of claim 87, wherein the CpG ODN is CpG ODN 1585, CpG ODN 2216, CpG ODN 2336, CpG ODN 1668, CpG ODN 1826, CpG ODN 2006, CpG ODN 2007, CpG ODN BW006, CpG ODN D-SL01, CpG ODN 2395, CpG ODN M362, or CpG ODN D-SL03.
